(19) United States
(12) Patent Application Publication
(10) Pub. No.: US 2019/0275040 A1
(43) Pub. Date: Sep. 12, 2019

(54) COMPOUNDS AND METHODS FOR TREATING OR PREVENTING ALZHEIMER'S DISEASE

(71) Applicant: YALE UNIVERSITY, New Haven, CT (US)

(72) Inventors: Stephen STRITTMATTER, Durham, CT (US); Erik GUNTHER, Branford, CT (US)

(21) Appl. No.: 16/335,183

(22) PCT Filed: Sep. 26, 2017
(86) PCT No.: PCT/US17/53418

§ 371 Date: Mar. 20, 2019

Related U.S. Application Data
(60) Provisional application No. 62/399,626, filed on Sep. 26, 2016.

(51) Int. Cl. A61K 31/506; A61K 31/5377; A61K 31/517; A61K 31/5383; A61K 31/553; A61K 31/505; A61K 31/551; A61K 31/4365; A61P 25/28

(52) U.S. Cl. CPC A61K 31/506; A61K 31/5377; A61K 31/517; A61K 31/5383; A61P 25/28; A61K 31/505; A61K 31/551; A61K 31/4365; A61K 31/553

(57) ABSTRACT

The present invention provides methods for treating Alzheimer's Disease, or preventing synaptic death associated with Alzheimer's Disease by administering a Pyk2 inhibitor. In certain embodiments, the Pyk2 inhibitor is specific for Pyk2. In other embodiments, the Pyk2 inhibitor also inhibits Fyn.

### CROSS-REFERENCE TO RELATED APPLICATIONS

The present application claims priority under 35 U.S.C. § 119(e) to U.S. Provisional Patent Application No. 62/399,626, filed Sep. 26, 2016, which application is incorporated herein by reference in its entirety.

### STATEMENT REGARDING FEDERALLY SPONSORED RESEARCH OR DEVELOPMENT

This invention was made with government support under AG034924 awarded by National Institutes of Health. The government has certain rights in the invention.

### BACKGROUND OF THE INVENTION

Alzheimer's Disease (AD) afflicts more than 5.4 million individuals in the U.S. alone, and there are over 46 million dementia patients worldwide. There are no available therapies to slow, halt or reverse the AD-associated neurodegeneration. AD is thought to be triggered by formation of amyloid-β (Aβ) oligomers. AD pathology includes formation of Aβ plaque and neurofibrillary Tau tangles, with neuronal loss and gliosis. Further, synaptic loss is consistent and pronounced in AD patients. Positron emission tomography (PET) and cerebrospinal fluid (CSF) biomarkers demonstrate early Aβ accumulation in AD patients, years prior to development of dementia. Later, increased CSF phospho-Tau is detected, and tangles develop histologically and spread topographically.

The early presence of Aβ oligomers in AD patients, along with early onset AD genetics, suggests Aβ oligomer formation is a trigger for AD development. Experimentally, specific Aβ conformations trigger neuronal dysfunction. Unfortunately, while pathology, biomarker and early onset dominant cases have demonstrated the role of Aβ peptide accumulation to trigger downstream neuroinflammation, hyperphosphorylated Tau tangle pathology and eventual cell loss, the biochemical steps and progression of AD remain poorly defined. For example, Phase III trials targeting Aβ have failed to meet their endpoints. This indicates that deeper pathophysiological understanding about AD is lacking.

Synapses are crucial for cognitive function, and synapse loss is amongst the most robust and consistent AD finding. Human AD-derived soluble Aβ oligomers (Aβo) exert synaptotoxic effects. Therefore, defining the mechanism by which pathological Aβo drive synaptic damage and subsequent events can highlight optimal sites for intervention. Human genetic studies of Late Onset AD (LOAD) have documented a range of common and rare risk factors with varying affect sizes. A number have been linked to neuroinflammation, endocytosis and protein processing. However, direct implication of documented risk genes in AD-specific molecular steps in neurons immediately proximal to synapse loss has remained nil.

There is thus a need in the art for identifying compounds and compositions that can be used to treat and/or prevent AD. The present disclosure addresses this need.

### BRIEF SUMMARY OF INVENTION

The invention provides a method of treating or preventing an Aβ-modulated disease or disorder in a mammal. The invention further provides a method of preventing synaptic death, or improving synaptic survival, associated with an Aβ-modulated disease or disorder in a mammal. The invention further provides a kit for preventing or treating an Aβ-modulated disease, or preventing synaptic death or improving synaptic survival, in a mammal.

In certain embodiments, the method comprises administering to the mammal a therapeutically effective amount of a Pyk2 inhibitor. In other embodiments, the Pyk2 inhibitor has a Ki≤100 nM against Pyk2. In yet other embodiments, the Pyk2 inhibitor is selective for Pyk2 over at least one other kinase and/or enzyme. In yet other embodiments, the Pyk2 inhibitor also inhibits Fyn.

In certain embodiments, the Fyn inhibitor is selected from the group consisting of a nucleic acid, siRNA, antisense nucleic acid, ribozyme, peptide, antibody, small molecule, antagonist, aptamer, peptidomimetic, and any combinations thereof. In other embodiments, the small molecule is selected from the group consisting of PF-719, tamatinib, foretinib, lestaurtinib, and Compounds 1-8, or a salt, solvate, tautomer, geometric isomer, enantiomer, and/or diastereoisomer thereof.

In certain embodiments, the inhibitor is the only therapeutically effective agent administered to the mammal. In other embodiments, the inhibitor is the only therapeutically effgective agent administered to the mammal in an amount sufficient to treat or prevent an Aβ-modulated disease or disorder in the mammal, and/or prevent synaptic death, or improve synaptic survival, associated with an Aβ-modulated disease or disorder in a mammal. In yet other embodiments, administration of the Pyk2 inhibitor provides a circulating Pyk2 inhibitor concentration of at least 100 nM in the mammal.

In certain embodiments, the Aβ-modulated disease or disorder is selected from the group consisting of Alzheimer's Disease (AD), prodromal Alzheimer's Disease, amnestic mild cognitive impairment (MCI), Down syndrome dementia, traumatic brain injury, and frontotemporal dementia. In other embodiments, the Aβ-modulated disease or disorder is AD. In yet other embodiments, Aβ oligomer-induced signaling is inhibited in the mammal. In yet other embodiments, the mammal is human. In yet other embodiments, the Pyk2 inhibitor is administered to the mammal by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes. In yet other embodiments, the mammal is further administered at least one additional agent that treats or prevents the Aβ-modulated disease or disorder in the mammal. In yet other embodiments, the inhibitor and at least one additional agent are coformulated.

In certain embodiments, the kit comprising a Pyk2 inhibitor, optionally an applicator, and optionally an instructional material for use thereof, wherein the instructional material recites the amount of, and frequency with which, the Pyk2 inhibitor is to be administered to the mammal.

### BRIEF DESCRIPTION OF THE DRAWINGS

The following detailed description of illustrative embodiments of the invention will be better understood when read in conjunction with the appended drawings. For the purpose of illustrating the invention, certain illustrative embodiments are shown in the drawings. It should be understood, however, that the invention is not limited to the precise arrangements and instrumentalities of the embodiments shown in the drawings.

FIG. 1 is a diagram depicting mGluR5 and Pyk2 at the center of an Aβo-cellular prion protein (PrPC)-Fyn cascade of AD synapse damage. Pyk2 variation is a genetic risk for late-onset AD (LOAD) and is centered within an AD transduction network. Schematic illustrates a role for mGluR5 in linking cell surface Aβo-PrPC complexes to intracellular Fyn. Proteins are clustered in the post-synaptic densities (PSD) and alter N-methyl D aspartate (NMDA) receptors (NMDA-Rs), calcium and protein translation. Tau plays a role in localizing Fyn. Aberrant PrPC-mGluR5-Fyn signaling leads to synaptic malfunction and loss. Pyk2 associates with mGluR5 and Fyn, and is regulated by calcium.

FIG. 2A depicts Hippocampal E 18 neurons transfected with a CAG-GFP vector to fill neurons with marker. At DIV21, spinning disc confocal images were collected, and then Aβo added. After 6 hours, the same region was reimaged, and spine loss during the incubation was scored, blind to treatment and genotype. FIG. 2B depicts the degree of spine loss during 6 hours with either vehicle or Aβo for WT and Pyk2−/− neurons. **P<0.005; ANOVA, mean±SEM, n=4 cultures.

FIG. 3A depicts WT slices that were treated with either vehicle or Aβo 30 min prior to theta burst stimulation (TBS). The slope of the field excitatory postsynaptic potential (fEPSP) is plotted as a function of time. Data are graphed as mean±SEM (n=6-12). Inset, representative traces immediately before TBS and 60 min post-TBS (red) are superimposed. FIG. 3B depicts WT and Pyk2−/− brain slices in the absence of Aβo, plotted as in FIG. 3A. FIG. 3C depicts average fEPSPs at 50-60 min post-TBS plotted for the indicated conditions as mean±SEM (n=3-20). Where indicated, 500 nM PF719 kinase inhibitor was pre-incubated with WT slices for 60 min prior to addition of Aβo or vehicle for 30 min, and then TBS. One-way ANOVA, **, p<0.01. Taken together, FIGS. 3A-3C show that Pyk2 loss of function rescues Aβo-induced impairment of long term potentiation (LTP).

FIG. 4A depicts the time mice spent interacting with a familiar and a novel object is plotted. Mean±SEM, n=10-16 mice. *, p<0.05; **, p<0.01 by two-way ANOVA, Sidak's multiple comparisons test. FIG. 4B depicts a passive avoidance paradigm. Vehicle-treated APP/PS1 mice showed a significant decrease in latency to enter the dark compartment in retention tests compared to WT, but senescence accelerated mice (SAM) treated performed equally to control (**, p<0.01 by two-way ANOVA with Tukey post hoc pairwise comparisons). Mean±SEM, n=10-16 mice. FIG. 4C depicts spatial learning and memory as examined in the Morris-Water-Maze after 30 swim trials to a hidden platform. A 60 second probe trial was performed 24 hours after completion of training Time spent in the quadrant where the hidden platform had been located. Mean±SEM of n=10-16 mice/group (*, p<0.05 by one-way ANOVA with Tukey post hoc pairwise test). Dashed line represents chance (no memory of target location). Taken together, FIG. 4A-4C demonstrate that Pyk2 loss of function rescues learning and memory deficits in APP/PS1 transgenic mice.

FIG. 5A plots the time mice spent interacting with a familiar and a novel object. Mean±SEM, n=10-16 mice. *, p<0.05; **, p<0.01 by two-way ANOVA, Sidak's multiple comparisons test. FIG. 5B depicts spatial learning and memory as examined by the Morris-Water-Maze after 30 swim trials to a hidden platform. A 60 second probe trial was performed 24 hours after completion of training. Time spent in the quadrant where the hidden platform had been located. Mean±SEM of n=7-23 mice. *, p<0.05; ***, p<0.001. Dashed lines in FIGS. 5A-5B are chance (no memory). In FIGS. 5A-5B, WT or APP/PS1 mice of 12 months were treated with PF-719 at 25 mg/kg/d minipump i.p. infusion for 3 weeks and then tested during continued drug administration. Control groups included both vehicle and untreated mice of the indicated genotypes. Taken together, FIGS. 5A-5B demonstrate that Pyk2 inhibition rescues learning and memory deficits in APP/PS1 transgenic mice.

FIG. 6A depicts Fyn inhibition by AZD0530, PF-719 and tamatinib in a luciferase-based biochemical assay. FIG. 6B depicts Pyk2 inhibition by AZD0530, PF-719 and tamatinib in a luciferase-based biochemical assay.

FIGS. 7A-7G illustrate the finding that activated Pyk2 is present in human AD, interacts with Fyn, and induces dendritic spine loss. FIG. 7A: TBS-T soluble human Alzheimer's disease brain extracts were immunoblotted with anti-Pyk2 or anti-pY402 Pyk2 specific antibodies. FIG. 7B: Densitometric analysis of the immunoblots from FIG. A. p-Pyk2/Pyk2 ratio values were normalized to control mean value. Data are graphed as mean±SEM (Control, n=10; AD, n=10). *p<0.05 by Student's two-tailed t test. FIG. 7C: Representative immunofluorescent images of Pyk2 immunoreactivity from hippocampal CA1 in WT and Pyk2 null mice at 12-months age. Scale bar=10 μm; confocal images were captured using a 63× objective. FIG. 7D: Hek293T cells were co-transfected with either control vector or flag-tagged Fyn and GFP-tagged Pyk2 or K457A kinase dead mutant. Lysates were immunoblotted with anti-GFP (for GFP tagged Pyk2 and K457A mutant), anti-Fyn, and anti-pSFK antibodies. FIG. 7E: Representative GFP fluorescent images of cultured mouse hippocampal neurons. Neurons were transfected with GFP alone, GFP-Pyk2, GFP-Pyk2 with 1 μM PF-719 (selective Pyk2 kinase inhibitor), or GFP-K457A at DIV 17 and then fixed at DIV 21. Enlarged images of the insets enclosed with rectangles on top images (bottom). Scale bars, low magnification (top): 50 μm; high magnification (bottom): 10 μm. FIG. 7F: Lysates from transfected neurons were subjected to western blotting with anti-GFP and anti-pY402 Pyk2 antibodies. FIG. 7G: Quantification of dendritic spine density in the transfected neurons. Data are presented as mean±SEM (GFP, n=10; GFP-Pyk2, n=11; GFP-Pyk2 & PF-719, n=9; GFP-K457A, n=10 coverslips from 3 different cultures). *p<0.05, ***p<0.001 by one-way ANOVA, Tukey's multiple comparisons test.

FIGS. 8A-8F illustrate the finding that Graf1c interacts with Pyk2 and co-localizes to postsynaptic terminal. FIG. 8A: Lysates from GFP, GFP-Pyk2, or GFP-K457A transfected Hek293T cells or the same lysates mixed with mouse brain lysate (Brain L) were immunoprecipitated with anti-GFP antibody. The immunoprecipitates were separated by SDS-PAGE and silver stained to identify proteins for subsequent LC-MS/MS analysis. Two major binding protein bands were identified as HSP90 and Graf1c, with the latter being specific to brain lysate samples. FIG. 8B: Graf1 isoforms domain structure diagrams and binding test in Pyk2 and Graf1 isoforms overexpressed Hek293T cells. Pyk2 and Graf1 isoforms co-transfected Hek293T cell lysates were immunoprecipitated with anti-GFP antibody and immunoblotted with anti-Graf1 and anti-GFP antibodies. FIG. 8C: WT and Pyk2−/− mouse brain lysates were immunoprecipitated with anti-Pyk2 antibody and immunoblotted with anti-Graf1 and anti-Pyk2 antibodies. FIG. 8D: Graf1c and indicated GFP or GFP tagged Pyk2 and mutants (PXXP1ₘᵤₜ, P714AP717A; PXXP2ₘᵤₜ, P857AP860A; ΔPRD, a.a. 679-870 deleted; PRD, a.a. 679-870) were co-transfected in Hek293T cell and then immunoprecipitated with anti-GFP antibody and immnoblotted with anti-GFP and anti-Graf1 antibodies. FIG. 8E: Cultured hippocampal neurons were transfected with GFP and RFP-Graf1c, GFP-Pyk2 and RFP, GFP-Pyk2 and RFP-Graf1c, or GFP-PXXP2ₘᵤₜ and RFP-Graf1c at DIV 17 and imaged at DIV 19 without fixation. High magnification images are enlarged view of the rectangle regions on top images (bottom). Scale bars, low magnification: 25 μm; high magnification: 10 μm. FIG. 8F: Cultured hippocampal neurons expressing GFP-PSD95 and RFP-Graf1c were imaged at DIV 19. Scale bars, low magnification: 25 μm; high magnification: 10 μm.

FIGS. 9A-9G illustrate the finding that postsynaptic Pyk2 localization is increased in Aβo-treated cultured neurons and APP/PS1 mice. FIG. 9A: Hippocampal neurons were incubated for 24 hours with vehicle (Veh) or Aβo (1 μM monomer, 10 nM oligomer estimate) in culture media then fixed and stained with anti-Pyk2 and anti-PSD-95 antibodies. Lower panels are enlarged images. Scale bars: 40 μm (Top), 10 μm (Bottom). FIG. 9B: Quantification of Pyk2 immunofluorescence in PSD-95 positive postsynaptic area. Data are graphed as mean±SEM (Veh 1 hour, n=11; Aβo 1 hour, n=10; Veh 6 hours, n=10; Aβo 6 hours, n=9; Veh 24 hours, n=10; Aβo 24 hours, n=9 coverslips from 3 different cultures). *p<0.05 by Student's two-tailed t test. FIGS. 9C-9D: Quantitative analysis of GFP-Pyk2 translocation by glutamate stimulation after Veh or Aβo treatment for 24 hours in cultured neurons. The translocation rates were calculated by fluorescence intensity change during 25 μM glutamate stimulation (recruitment, FIG. 9C) and redistribution after glutamate washout using 10 μM CNQX and 50 μM APV in Tyrode's solution (recovery, FIG. 9D). Half times (t_{1/2}) were calculated by fitting the fluorescence intensity traces during recruitment and recovery period with a single exponential function. Mean±SEM (n=8 coverslips from 3 different cultures). *p<0.05 by Student's two-tailed t test. See also FIGS. 15A-15F. FIG. 9E: Total supernatant 1 (S1) and PSD fractions (20 μg proteins) from 9 month WT or APP/PS forebrain were subjected to western blotting with anti-Pyk2, anti-Graf1, anti-PSD95, and actin antibodies. FIGS. 9F-9G: Quantification of Graf1c (FIG. 9F) and Pyk2 (FIG. 9G) levels in each fraction normalized to PSD-95. Mean±SEM (n=3 mice). *p<0.05 by Student's two-tailed t test.

FIGS. 10A-10F illustrate the finding that Pyk2 induces RhoA activation and dendritic spine loss through Graf1c interaction. FIG. 10A: GST-RBD pull-down assay from Hek293T cell lysate from cells expressing indicated plasmids. The proteins retained by GST-RBD-immobilized beads and lysates were subjected to immunoblotting with anti-Myc, anti-Pyk2, anti-Graf1 antibodies. FIG. 10B: Levels of RhoA-GTP (RBD pull-down) and RhoA-total (5% lysate) were quantified by densitometric measurement. Y axis represents the relative ratio in indicated cells compared to in Myc-RhoA only expressing cells. Data are graphed as mean±SEM (n=3 experiments). *p<0.05, **p<0.01 by one-way ANOVA, Tukey's multiple comparisons test. FIG. 10C: GST-RBD pull-down assay of forebrain lysates from 6 or 9-month WT and APP/PS1 mice. The pull-down proteins and lysates were subjected to immunoblotting with anti-RhoA antibody. The bait proteins amount was confirmed by Coomassie blue staining from pull-downed samples. FIG. 10D: Quantification of RhoA-GTP/total RhoA ratio by densitometric analysis. The ratio was normalized to age-matched WT. Data are graphed as mean±SEM (n=6 mice for each condition). **p<0.01 by Student's two-tailed t test. FIG. 10E: Representative GFP fluorescence images of the cultured hippocampal neurons. Neurons were transfected with GFP, GFP-Pyk2, GFP-PXXP2ₘᵤₜ, GFP-Pyk2 and 10 μM Y27632 co-treatment, or GFP-Pyk2 and Myc-RhoA-T19N at DIV 17 and then fixed at DIV 21. The Myc-RhoA-T19N expression was examined by immunostaining with anti-Myc antibody. Scale bar is 10 μm. FIG. 10F: Quantification of dendritic spine density. Data are graphed as mean±SEM (GFP, n=7; GFP-Pyk2, n=9; GFP-PXXP2ₘᵤₜ, n=7; GFP-Pyk2 & Y27632, n=7; GFP-Pyk2 & Myc-RhoA-T19N, n=5 coverslips from 3 different cultures). ***p<0.001 by ANOVA, Tukey's multiple comparisons test.

FIGS. 11A-11H illustrate the finding that Aβo-induced dendritic spine deficits are prevented by deletion of Pyk2 and RhoA inhibition in primary neurons. FIG. 11A: Representative images of dendritic spine motility by standard deviation projection of time-lapse image. Myristoyl-GFP expressing hippocampal neurons (DIV21) from WT or Pyk2−/− mice were imaged for 5 minutes with 10 second intervals after Veh or Aβo (1 μM monomer, 10 nM oligomer estimate) preincubation with or without 1 μM PF-719, RFP-Pyk2-PRD (PRD), or 10 μM Y27632 for 24 hours. The standard deviation value for each pixel is shown by thermal color scale. Scale bar, 10 μm. FIG. 11B: Quantification of spine motility, expressed as % of changed area of spines with subtraction of changed area of dendritic shaft as background (changed area=(ΔAₛₚᵢₙₑ/Aₛₚᵢₙₑ×100)−(ΔA_{shaft}/A_{shaft}×100)). Data are graphed as mean±SEM (Veh in WT, n=16; Aβo in WT, n=14; Veh in Pyk2^{-/-}, n=14; Aβo in Pyk2^{-/-}, n=16; Veh in WT & PF-719, n=17; Aβo in WT & PF-719, n=16; Veh in WT & Pyk2-PRD, n=6; Aβo in WT & Pyk2-PRD, n=6; Veh in WT & Y27632, n=15; Aβo in WT & Y27632, n=11 neurons from 3 different cultures). ***p<0.001 by Student's two-tailed t test. FIGS. 11C & 11E: Representative images of DIV 21 GFP transfected neurons from WT mice (FIG. 11C) or Pyk2−/− mice (FIG. 11E) after non-treated (Con), Veh, or Aβo treatment for 6 h or 4 days. FIGS. 11D & 11F: Quantification of dendritic spine density for conditions shown in FIG. 11C & FIG. 11E. Data are graphed as mean±SEM (n=7-8 coverslips from 3 different cultures). **p<0.01, ***p<0.001 by ANOVA, Tukey's multiple comparisons test. FIGS. 11G & 11H: GFP, GFP-Pyk2-PRD, or GFP & Myc-RhoA-T19N transfected neurons were incubated with Veh or Aβo for 4 days and then fixed and stained with anti-Myc antibodies at DIV 21. Mean±SEM (n=5-8 coverslips from 3 different cultures). One-way ANOVA, Tukey's multiple comparisons test.

FIGS. 12A-12D illustrate the finding that Pyk2 mediates learning and memory deficits in APP/PS1 mice. FIG. 12A: 12-month-old mice and age-matched litter mate controls were subjected to MWM test. Latency was calculated as the time to find the hidden platform across 6 blocks of 4 trials. APP/PS1 mice (n=12) spent significantly more time to find the hidden platform compared to WT (n=11), Pyk2−/− (n=9), or APP/PS1; Pyk2−/− mice (n=9). Data are graphed as mean±SEM, two-way RM-ANOVA with Tukey's multiple comparison test: *p<0.05 and ***p<0.001. FIG. 12B: The probe trial was performed 24 hours after the 6^{th} forward swim trial block by removing the hidden platform. Quadrant time (%) is calculated as % time spent in the target quadrant in one minute. APP/PS1 (n=12) mice spend significantly less time in the target quadrant compared to WT (n=11), Pyk2−/− (n=9), or APP/PS1; Pyk2−/− mice (n=9). Data are graphed as mean±SEM, one-way ANOVA with Tukey's multiple comparison test: *p<0.05 and **p<0.01. FIG. 12C: Mice were subjected to NOR test by familiarizing them to an object and subsequently allowed to explore a novel object and familiar one hour after exposure to the familiar object. APP/PS1 mice (n=14) did not display a preference for either familiar or novel object, while WT (n=11), Pyk2−/− (n=9), and APP/PS1; Pyk2−/− mice (n=10) spent significantly more time with the novel object. Data are graphed as mean±SEM, two-way ANOVA with Tukey's multiple comparison test: *p<0.05, **p<0.01, and ****p<0.0001. FIG. 12D: Mice were subjected to PAT and latency to enter the dark chamber was measured initially and 24 hours after receiving a mild foot shock in the dark chamber. APP/PS1 mice (n=16) re-entered the dark chamber significantly more rapidly compared to WT (n=14), Pyk2−/− (n=11), or APP/PS1; Pyk2−/− mice (n=10). Data are graphed as mean±SEM, two-way ANOVA with Tukey's multiple comparison test: *p<0.05 and **p<0.01.

FIGS. 13A-13G illustrate the finding that Pyk2 mediates Aβo-dependent deficit in LTP and rescues synapse loss in APP/PS1 mice. FIGS. 13A, 13C, & 13D: Acute brain slices from WT or Pyk2−/− mice were treated with vehicle or Aβo and used to record field excitatory postsynaptic potentials (fEPSPs) from the CA3-CA1 hippocampal circuit. The slope of the fEPSPs is plotted as a function of time. Representative traces before theta burst stimulation in black (TBS, denoted by black arrowhead at time=0) and at 60 min post-TBS in red are superimposed (average, 6 sweeps) in the respective graphs. Data are graphed as mean±SEM of averaged slices per group. Slices were treated with Vehicle (F12 without: Glutamate, Glycine, and Phenol Red) or Aβo (1 μM monomer, 10 nM oligomer estimate) for 30 min prior to TBS. (FIG. 13B) WT slices treated with Aβo (n=14) showed a significant decrease in fEPSP compared to WT slices treated with vehicle (n=22), Pyk2−/− slice treated with vehicle (n=11), or Pyk2−/− slices treated with Aβo (n=10). Data are graphed as mean±SEM, n=one slice; two-way ANOVA with Fisher's LSD multiple comparison test: *p<0.05. FIG. 13E: Representative immunofluorescent images of immunoreactive SV2A in the dentate gyrus for the indicated genotypes. Scale bar=10 μm. FIG. 13F: Quantification of % area immunoreactive for SV2A using an anti-SV2A antibody. WT mice (n=8), Pyk2−/− (n=8), and APP/PS1; Pyk2−/− (n=8) mice have significantly higher SV2A-immunoreactive % area compared to APP/PS1 mice (n=8). Data are graphed as mean±SEM, n=one mouse (from three 40 μm sections averaged per mouse); One-way ANOVA with Tukey's multiple comparison test: *p<0.05, ****p<0.0001. FIG. 13G: Quantification of % area immunoreactive for PSD-95 using an anti-PSD-95 antibody. Pyk2−/− (n=8) and APP/PS1; Pyk2−/− (n=8) mice have significantly higher PSD-95-immunoreactive % area compared to APP/PS1 mice (n=8) with WT (n=8) mice displaying a modest increase in PSD-95 immunoreactivity compared to APP/PS1 mice. Data are graphed as mean±SEM, n=one mouse (from three 40 μm sections averaged per mouse); One-way ANOVA with Uncorrected Fisher's LSD multiple comparison test: *p<0.05.

FIGS. 14A-14F illustrate the finding that Pyk2 is predominantly expressed in neurons and co-immunoprecipitates with Fyn. FIGS. 14A-14D: Immunohistology was performed on age-matched littermate controls from WT, Pyk2−/−, and APP/PS1 mice at 12-months of age. Scale bar=10 μm; confocal images were captured using a 63× objective. Representative immunofluorescent images of immunoreactive Pyk2 (same as FIG. 7C) costained with MAP2 in CA1 (FIG. 14A), Pyk2 and PSD95 in CA1 (FIG. 14B), Pyk2 and Iba1 in the Stratum radiatum (FIG. 14C), and Pyk2 and GFAP in the Stratum radiatum (FIG. 14D). FIGS. 14E-14F: Hek293T cells were co-transfected with flag-tagged Fyn and GFP alone, GFP-tagged Pyk2, or K457A mutant. Lysates were immunoprecipitated with anti-GFP (FIG. 14E) or anti-flag (FIG. 14F) antibodies and immunoblotted with anti-flag and anti-GFP antibodies.

FIGS. 15A-15F illustrate the finding that Graf1 regulates Pyk2 localization in postsynaptic terminals in cultured neuron. FIG. 15A: Cultured Hippocampal neurons were transfected with either U6 vector (U6 Control), Graf1, or Graf1 a, b specific shRNA by electroporation before plating. Lysates from DIV 21 transfected neurons were immunoblotted with anti-Graf1 and anti-actin antibodies. * indicates non-specific band. FIG. 15B: Immunohistology of DIV21 Control and Graf1 shRNA transfected neurons co-immunostained with anti-Pyk2 (Red) and anti-PSD-95 (Blue) antibodies. Transfected neurites were marked with white dashed line based on GFP fluorescence. Scale bar, 10 μm. FIG. 15C: Quantification of postsynaptic terminal localized Pyk2 levels. To test the Graf1 knockdown (KD) effect on Pyk2 synaptic localization, GFP and PSD-95 double positive areas were selected using Volocity software automatically and then measure the mean intensity of Pyk2 in each selected area. Mean±SEM (U6 Control, n=22; Graf1 shRNA, n=21; n=separate neuron). *p<0.05 by Student's two-tailed t test. FIG. 15D: Pyk2-PRD competes with Pyk2 and inhibits Graf1c interaction in overexpressed Hek293T cells. Lysates from indicated plasmids transfected Hek293T cells were immunoprecipitated with anti-HA antibodies. The input lysates (5%) and precipitates were subjected to western blotting with anti-Graf1 and Pyk2 PRD region specific anti-Pyk2 antibodies. FIG. 15E: GFP or GFP-Pyk2-PRD (GFP-PRD) expressed neurons were co-stained with Pyk2 N-terminal specific (a.a. 1-100) anti-Pyk2 antibody (Red) and PSD-95 antibody (Blue). Transfected neurites were marked with white dashed line based on GFP fluorescence. Scale bar, 10 μm. FIG. 15F: Quantification of postsynaptic terminal localized Pyk2 levels with same method as FIG. 15C. Mean±SEM (GFP, n=19; GFP-PRD, n=14 neurons). *p<0.05 by Student's two-tailed t test.

FIGS. 16A-16E illustrate the finding that Aβo increases Pyk2 translocation dwell time in postsynaptic terminal after glutamate stimulation. FIG. 16A: Schematic diagram of experiment protocol. DIV 19-20 RFP and GFP-Pyk2 co-expressing hippocampal neurons were incubated with Veh or Aβo (1 μM monomer, 10 nM oligomer estimate) for 24 hours and then imaged for 2.5 minutes with 15 second intervals after 25 μM glutamate stimulation within Tyrode's solution (recruitment imaging). After recruitment imaging, neurons were perfused with 10 μM APV and 50 μM CNQX in Tyrode's solution for one minute and subsequently the recovery imaging was acquired for 25 min with 1 min intervals. FIGS. 16B & 16D: Representative time-lapse images of GFP-Pyk2 translocation after glutamate treatment (FIG. 16B) and redistribution after glutamate washout (FIG. 16D). RFP and GFP-Pyk2 merged images were presented in left side for first time frame and right side for last time frame. The Pyk2-GFP images were converted to gray scale for better visibility. Scale bar is 10 μm. FIGS. 16C & 16E: Normalized average fluorescence intensity profiles of recruitment time-lapse images (FIG. 16C) and recovery time-lapse images (FIG. 16E). Recruitment and Recovery values were normalized to average value of first 3 time points of recruitment images. Data are presented as mean±SEM (n=8 coverslips from 3 different cultures).

FIGS. 17A-17B illustrate the finding that Graf1 is a functional GTPase activating protein (GAP) in mature neurons and regulates dendritic spine formation. FIG. 17A: Representative fluorescence images of cultured hippocampal neurons. Neurons were transfected with U6 vector (Control), Graf1 shRNA, Graf1 shRNA and 10 μM Y27632, or Graf1 shRNA and Myc-RhoA-T19N at DIV 14 and then fixed at DIV 21. Scale bar, 10 μm. FIG. 17B: Quantification of dendritic spine density in the transfected neurons. Data are graphed as mean±SEM (Control, n=8; Graf1 shRNA, n5=5; Graf1 shRNA & Y27632, n=5; Graf1 shRNA & RhoA-T19N, n=5 coverslips from 3 different cultures). **p<0.01, ***p<0.001 by ANOVA, Tukey's multiple comparisons test.

FIGS. 18A-18H illustrate spine motility regulated by actin polymerization and normal synaptic markers in Pyk2−/− mice. FIG. 18A: Representative images of dendritic spine motility by standard deviation projection of time stacks. Hippocampal neurons (DIV21) expressing myristoyl-GFP were imaged for 5 minutes with 10 second intervals as a control and then incubated with 1 μm Cytochalasin D for 20 minutes, imaged again with same acquisition protocol as control image at the same neuron. To display the spine motility in time-lapse images, the time stack images over 5 min of each conditions were projected to standard deviation using Image J and color coated with thermal color scale. Scale bar, 10 μm. FIG. 18B: Quantification of spine motility, expressed as % of changed area of spines with subtraction of dendritic shaft area change (changed area=(ΔAₛₚᵢₙₑ/Aₛₚᵢₙₑ×100)−(ΔA_{shaft}/A_{shaft}×100)). Mean±SEM (n=5 coverslips). FIG. 18C: Pyk2 expression test in WT and Pyk2−/− mice forebrains. Lysates (1% Triton x-100, 150 mM NaCl, 50 mM Tris, Protease and Phosphatase inhibitor cocktails) from 6-month WT and Pyk2−/− mice forebrains were subjected to immunoblotting with anti-Pyk2 antibody which recognize N-terminus region (a.a. 1-100) of Pyk2. FIGS. 18D-18F: Synaptic protein expression profile in cortex and hippocampus from WT and Pyk2−/− mice. 1% SDS soluble cortex and hippocampus homogenates (homogenate with Syn-PER, 50 μg protein) were immunoblotted with anti-Pyk2, anti-PSD-95, anti-pNR2B (pY1472), anti-NR2B, anti-pSFK, anti-Fyn, and anti-actin specific antibodies (FIG. 18D). Quantification of protein levels by densitometric analysis in cortex (FIG. 18E) and hippocampus (FIG. 18F). All the protein levels were normalized to actin and then WT values. Data are graphed as mean±SD (n=3). pSFK/Fyn reduction in cortex p=0.1282, Student's two-tailed t test. FIGS. 18G-18H: Synaptic protein expression profile in hippocampal PSD fraction of WT and Pyk2−/− mice. PSD fraction (20 μg protein) was subjected to immunoblotting with indicated antibodies (FIG. 18G) and quantified by densitometric analysis (FIG. 18H). Mean±SD (n=3 mice). Student's two-tailed t test.

FIGS. 19A-19F illustrate the finding that deletion of Pyk2 limits astrocytosis but has no effect on microgliosis or Aβ plaque burden. FIG. 19A: Representative immunofluorescent images of anti-GFAP staining in the hippocampus for the indicated genotypes. Scale bar=50 μm. FIG. 19B: Quantification of % area immunoreactive for the astrocyte marker GFAP using an anti-GFAP antibody. APP/PS1 (n=8) mice have significantly more GFAP-immunoreactive % area compared to WT (n=8) and Pyk2−/− (n=8) mice, while APP/PS1; Pyk2−/− (n=6) mice have significantly less immunoreactive % area than APP/PS1 mice but significantly more than WT mice. Data are graphed as mean±SEM, n=separate mice (with three 40 μm sections averaged per mouse); One-way ANOVA with Tukey's multiple comparison test: *p<0.05, ***p<0.001, ****p<0.0001. FIG. 19C: Representative immunofluorescent images of Iba1 in the hippocampus for the indicated genotypes. FIG. 19D: Quantification of % area immunoreactive for the microglial marker Iba1 using an anti-Iba1 antibody. APP/PS1 (n=8) and APP/PS1; Pyk2−/− (n=6) mice have significantly more Iba1-immunoreactive % area compared to WT (n=8) and Pyk2−/− (n=8) mice. Data are graphed as mean±SEM, n=separate mice (with three 40 μm sections averaged per mouse); One-way ANOVA with Tukey's multiple comparison test: *p<0.05, **p<0.01. FIG. 19E: Representative immunofluorescent images of immunoreactive Aβ in the hippocampus for the indicated genotypes. Scale bar=200 μm. FIG. 19F: Quantification of % area immunoreactive for Aβ using an anti-Aβ antibody. APP/PS1 (n=8) and APP/PS1; Pyk2−/− (n=6) mice do not have significantly different Aβ-immunoreactive % area. Data are graphed as mean±SEM, n=separate mice (with three 40 μm sections averaged per mouse); Unpaired two-tailed t-test.

FIGS. 20A-20F illustrate the finding that Pyk2 mediates long-term depression in the CA1 of hippocampal slices. FIG. 20A: Acute brain slices from WT or Pyk2−/− mice were used to record field excitatory postsynaptic potentials (fEPSPs) from the CA3-CA1 hippocampal circuit. The slope of the fEPSPs is plotted as a function of time. Representative traces before a 5 minute low frequency stimulus (LFS) in black (LFS, denoted by black arrowhead at time=0) and at 60 min post-LFS in red are superimposed (average, 6 sweeps). FIG. 20B: WT (n=8) slices did not show a significant difference in fEPSP during the last 20 min of recording compared to Pyk2−/− slices (n=7). Data are graphed as mean±SEM, n=separate slices from three mice; Unpaired two-tailed t-test. FIG. 20C: Similar to FIG. 20A, hippocampal slices were used to record fEPSPs. Representative traces before a 15 minute LFS in black (LFS, denoted by black arrowhead at time=0) and at 60 min post-LFS in red are superimposed (average, 6 sweeps). FIG. 20D: WT (n=13) slices displayed a significant decrease in fEPSP during the last 20 min of recording compared to Pyk2−/− slices (n=12). Data are graphed as mean±SEM, n=separate slices; Unpaired two-tailed t-test: *p<0.05. FIG. 20E: Input-output (I/O) responses were graphed as fEPSPs (mV/ms) with respect to stimulus intensity (V) at 40 μs duration. No change was observed in baseline synaptic transmission from WT (n=9) and Pyk2−/− (n=9) slices by two-way ANOVA with Sidak's multiple comparison test. FIG. 20F: Paired-pulse ratios (fEPSP2ₛₗₒₚₑ/fEPSP1ₛₗₒₚₑ) in WT (n=9) and Pyk2−/− (n=9) slices were not significantly different by two-way ANOVA with Sidak's multiple comparison test.

### DETAILED DESCRIPTION OF THE INVENTION

The present invention relates in part to the discovery that Pyk2 helps mediate the pathological signaling associated with Aβ oligomers (Aβo). In certain embodiments, inhibition of Pyk2 inhibits Aβo signaling. Thus, the present invention relates generally to compositions and methods for treating and preventing an Aβ-modulated disease. Non-limiting examples of Aβ-modulated disease that are treatable or preventable with the compositions and methods of the present invention include, but are not limited to, Alzheimer's Disease (AD), prodromal Alzheimer's Disease, amnestic mild cognitive impairment (MCI), Down syndrome dementia, traumatic brain injury (TBI), and frontotemporal dementia.

Pyk2 (also PTK2B or FAK2) is a LOAD risk gene with direct effects on synaptic plasticity. Pyk2 is localized to post-synaptic densities (PSDs), where it regulates synaptic plasticity. As demonstrated herein, Pyk2 is activated in human AD brain and expressed selectively in neurons, where its activation reduces dendritic spine number. In brain, a major partner of Pyk2 is Graf1, a RhoA GTPase activating protein (GAP) inhibited by Pyk2. The ability of Aβo to reduce dendritic spine motility, to cause spine loss and to suppress LTP requires Pyk2 expression in vitro. AD model transgenic mice lacking Pyk2 were protected from transgene-induced synapse loss and memory impairment. Thus, the LOAD risk gene Pyk2 is coupled to an Aβo signaling pathway and is a proximal mediator of synapse loss.

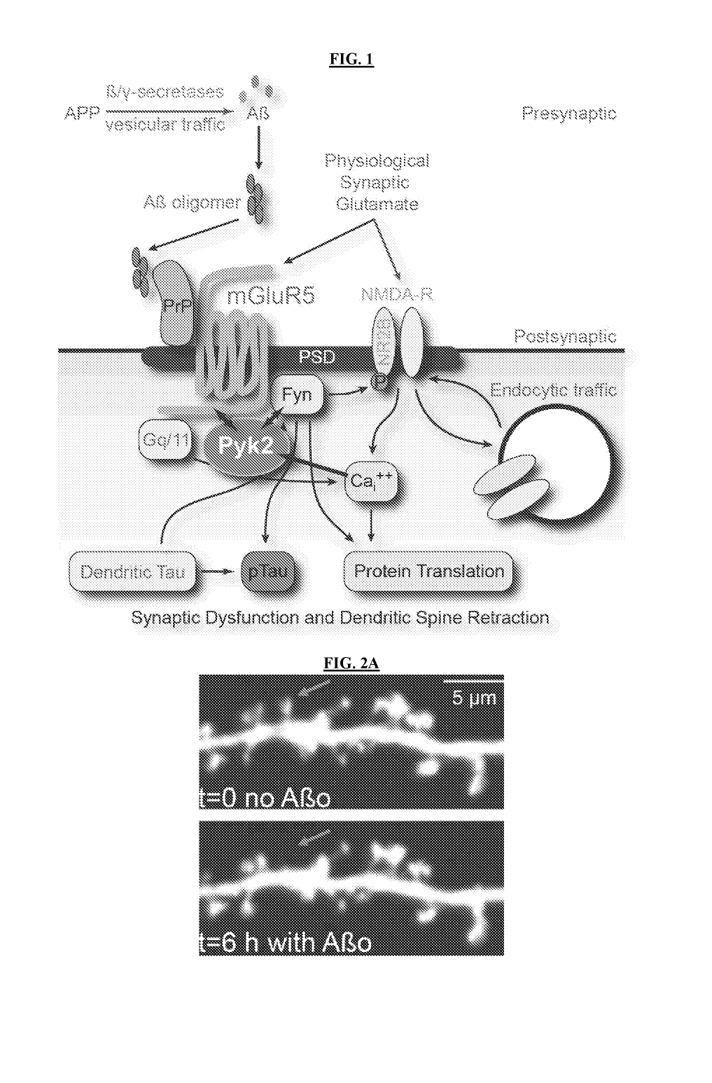

Aβo interacts with the neuronal surface to trigger post-synaptic pathology (FIG. 1). Cellular Prion Protein (PrPC) was identified in the only reported genome-wide unbiased screen for Aβo binding sites. Aβ binding to PrPC is of high affinity and is oligomer-specific, with no affinity for fibrillary or monomeric states. PrPC is not essential for certain Aβ-related phenotypes, but is required for cell death in vitro, reduced survival of APP/PS1 transgenic lines, electroencephalogram (EEG) discharges, synapse loss, 5HT axon degeneration, and learning and memory deficits. Critically, human AD brain-derived Aβ suppression of synaptic plasticity requires PrPC, and human AD contains PrPC-interacting Aβo species and Aβ-PrPC complexes. Levels of PrPC-interacting Aβo match memory loss in AD mouse models. Fyn tyrosine kinase activation is critical for Aβo action, and PrPC and Fyn are co-enriched in the PSD. Fyn inhibition has been proposed to prevent or delay disease progression. Indeed, the Src family kinase inhibitor AZD0530 (saracatinib) prevents Aβo-induced Fyn signaling, rescuing memory deficits and synapse loss in APP/PS1 mice. The metabotropic glutamate receptor, mGluR5, provides coupling between Aβo-PrPC and intracellular cascades. While negative allosteric modulators (NAMs) of mGluR5 rescue Aβo and AD transgene phenotypes, the therapeutic window is narrow because minor dose increases interrupt Glu signaling and impair behavior. The optimal therapeutic compound would preserve endogenous mGluR5 signaling from Glu, but block pathophysiological signaling from Aβo-PrPC.

Pyk2 is activated by intracellular calcium and is phosphorylated by Fyn to achieve full activation. The interaction of Pyk2 and Fyn is direct, bidirectional and synergistic: the two kinases associate and synergistically co-activate one another. Fyn inhibition suppresses Pyk2 activation and blocks Aβo stimulation. Transgenic mice have a two-fold increase of activated p-Pyk2. Treatment with AZD0530 reduces this level to that of WT mice. These data indicate that Aβo signals to Fyn and Pyk2 in AD. Pyk2 is thus a target for developing AD therapy (Table 1).

In certain embodiments, the present invention provides a composition for treating an Aβ-modulated disease in a subject, wherein the composition comprises a Pyk2 activity inhibitor. In other embodiments, the present invention provides a composition for treating an Aβ-modulated disease in a subject, wherein the composition comprises a Pyk2 expression inhibitor. Hereinafter, the phrase “Pyk2 inhibitor” can refer to either a Pyk2 activity inhibitor and/or a Pyk2 expression inhibitor, or any other type of Pyk2 inhibitor.

TABLE 1
Comparison of Aβo synapto-toxicity pathway therapeutic targets for AD

| | | | | | | Score as |
|---|---|---|---|---|---|---|
| | | | | Cognitive | | Therapy |
| | | | Drug | function | Genetic | (adding |
| | Genes in | Druggable | selectivity in | intact with | linkage to | ‘Yes’ |
| Target | Family | at low nM? | family? | inhibition? | LOAD? | columns) |
| Prp^{C} | One | No | Yes | Yes | No | 2 of 4 |
| | | protein-protein | sole member | KO is benign | | |
| Fyn | 9 | Yes | No | No | No | 1 of 4 |
| | Src's | Phase 2a trial | all SFKs | KO impaired | | |
| mGluR5 | 8 | Yes | Yes | No | No | 2 of 4 |
| | mGluRs | | | KO + NAM | | |
| | | | | abnl | | |
| Pyk2 | 2 | Yes | Yes | Yes | Yes | 4 of 4 |
| | FAK | | | KO is benign | | |

In certain embodiments, the Pyk2 inhibitor comprises a nucleic acid, siRNA, antisense nucleic acid, ribozyme, peptide, antibody, small molecule, antagonist, aptamer, peptidomimetic, and any combinations thereof, that reduces the activity of Pyk2.

In certain embodiments, the Pyk2 inhibitor has a Kᵢ≤about 100 nM against Pyk2. In other embodiments, the Pyk2 inhibitor has a Kᵢ≤about 90 nM, ≤about 80 nM, ≤about 70 nM, Kᵢ≤about 60 nM, ≤about 50 nM, ≤about 40 nM, Kᵢ≤about 30 nM, K; ≤about 20 nM, ≤about 10 nM, ≤about 8 nM, ≤about 6 nM, ≤about 4 nM, ≤about 2 nM, Kᵢ≤about 1 nM, Kᵢ≤about 0.5 nM, Kᵢ≤about 0.25 nM, Kᵢ≤about 0.1 nM, ≤about 0.05 nM, Kᵢ≤about 0.025 nM, or Kᵢ≤about 0.01 nM against Pyk2.

In certain embodiments, the Pyk2 inhibitor selectively inhibits Pyk2 over at least one other enzyme and/or kinase, such as but not limited to FAK (focal adhesion kinase), wherein the Kᵢ for that at least one enzyme and/or kinases is equal to or greater than about 5 times higher than its Kᵢ for Pyk2. In other embodiments, the Kᵢ for that at least one enzyme and/or kinases is equal to or greater than about 10, 15, 20, 25, 30, 35, 40, 45, 50, 75, 100, 200, 300, 400, 500, 600, 700, 800, 900, or 1,000 times higher than its Kᵢ for Pyk2. In yet other embodiments, the Pyk2 inhibitor inhibits FAK with a Kᵢ that is equal to or greater than about 5 times higher than its Kᵢ for Pyk2.

In certain embodiments, the Pyk2 inhibitor has a Kᵢ≤about 100 nM against Fyn. In other embodiments, the Pyk2 inhibitor has a Kᵢ≤about 90 nM, ≤about 80 nM, ≤about 70 nM, Kᵢ≤about 60 nM, ≤about 50 nM, ≤about 40 nM, Kᵢ≤about 30 nM, K; ≤about 20 nM, Kᵢ≤about 10 nM, ≤about 8 nM, ≤about 6 nM, ≤about 4 nM, ≤about 2 nM, Kᵢ≤about 1 nM, ≤about 0.5 nM, ≤about 0.25 nM, K≤about 0.1 nM, ≤about 0.05 nM, Kᵢ≤about 0.025 nM, or Kᵢ≤about 0.01 nM against Fyn.

In certain embodiments, the inhibitor inhibits both Pyk2 and Fyn, and the ratio of Ki(Pyk2)/Ki(Fyn) for the inhibitor ranges from a minimum ratio to a maximum ratio. In other embodiments, the minimum ratio is about 1:15. In yet other embodiments, the minimum ratio is about 1:14. In yet other embodiments, the minimum ratio is about 1:13. In yet other embodiments, the minimum ratio is about 1:12. In yet other embodiments, the minimum ratio is about 1:11. In yet other embodiments, the minimum ratio is about 1:10. In yet other embodiments, the minimum ratio is about 1:9. In yet other embodiments, the minimum ratio is about 1:8. In yet other embodiments, the minimum ratio is about 1:7. In yet other embodiments, the minimum ratio is about 1:6. In yet other embodiments, the minimum ratio is about 1:5. In yet other embodiments, the minimum ratio is about 1:4. In yet other embodiments, the minimum ratio is about 1:3. In yet other embodiments, the minimum ratio is about 1:2. In yet other embodiments, the maximum ratio is about 15:1. In yet other embodiments, the maximum ratio is about 14:1. In yet other embodiments, the maximum ratio is about 13:1. In yet other embodiments, the maximum ratio is about 12:1. In yet other embodiments, the maximum ratio is about 11:1. In yet other embodiments, the maximum ratio is about 10:1. In yet other embodiments, the maximum ratio is about 9:1. In yet other embodiments, the maximum ratio is about 8:1. In yet other embodiments, the maximum ratio is about 7:1. In yet other embodiments, the maximum ratio is about 6:1. In yet other embodiments, the maximum ratio is about 5:1. In yet other embodiments, the maximum ratio is about 4:1. In yet other embodiments, the maximum ratio is about 3:1. In yet other embodiments, the maximum ratio is about 2:1. In yet other embodiments, the ratio of Ki(Pyk2)/Ki(Fyn) for the inhibitor is about 1:1.

In certain embodiments, the ratio of Ki(Pyk2)/Ki(Fyn) for the inhibitor ranges from about 1:15 to about 15:1. In other embodiments, the ratio of Ki(Pyk2)/Ki(Fyn) for the inhibitor ranges from about 1:14 to about 14:1. In yet other embodiments, the ratio of Ki(Pyk2)/Ki(Fyn) for the inhibitor ranges from about 1:13 to about 13:1. In yet other embodiments, the ratio of Ki(Pyk2)/Ki(Fyn) for the inhibitor ranges from about 1:12 to about 12:1. In yet other embodiments, the ratio of Ki(Pyk2)/Ki(Fyn) for the inhibitor ranges from about 1:11 to about 11:1. In yet other embodiments, the ratio of Ki(Pyk2)/Ki(Fyn) for the inhibitor ranges from about 1:10 to about 10:1. In yet other embodiments, the ratio of Ki(Pyk2)/Ki(Fyn) for the inhibitor ranges from about 1:9 to about 9:1. In yet other embodiments, the ratio of Ki(Pyk2)/Ki(Fyn) for the inhibitor ranges from about 1:8 to about 8:1. In yet other embodiments, the ratio of Ki(Pyk2)/Ki(Fyn) for the inhibitor ranges from about 1:7 to about 7:1. In yet other embodiments, the ratio of Ki(Pyk2)/Ki(Fyn) for the inhibitor ranges from about 1:6 to about 6:1. In yet other embodiments, the ratio of Ki(Pyk2)/Ki(Fyn) for the inhibitor ranges from about 1:5 to about 5:1. In yet other embodiments, the ratio of Ki(Pyk2)/Ki(Fyn) for the inhibitor ranges from about 1:4 to about 4:1. In yet other embodiments, the ratio of Ki(Pyk2)/Ki(Fyn) for the inhibitor ranges from about 1:3 to about 3:1. In yet other embodiments, the ratio of Ki(Pyk2)/Ki(Fyn) for the inhibitor ranges from about 1:2 to about 2:1. In yet other embodiments, the ratio of Ki(Pyk2)/Ki(Fyn) for the inhibitor is about 1:1.

In certain embodiments, the Pyk2 inhibitor comprises one or more of the following compounds, or a salt, solvate, tautomer, geometric isomer, enantiomer, or diastereoisomer thereof:
PF-719 (or 4-(((6-ethoxybenzo[d]thiazol-2-yl)thio)methyl)-6-morpholino-1,3,5-triazin-2-amine):

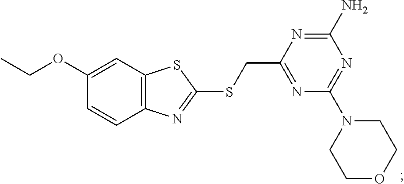 [SMILES: CCOc1ccc2nc(SCc3nc(N)nc(N4CCOCC4)n3)sc2c1]

tamatanib (or 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one):

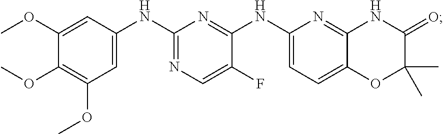

tamatanib phosphate (or fostamatinib, or [6-({5-Fluoro-2-[(3,4,5-trimethoxyphenyl)amino] pyrimidin-4-yl}amino)-2,2-dimethyl-3-oxo-2,3-dihydro-4H-pyrido[3,2-b][1,4]oxazin-4-yl]methyl dihydrogen phosphate:

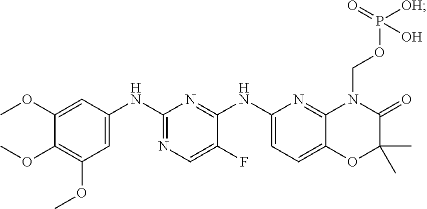

foretinib (or N-(3-fluoro-4-((6-methoxy-7-(3-morpholinopropoxy)quinolin-4-yl)oxy)phenyl)-N-(4-fluorophenyl)cyclopropane-1,1-dicarboxamide):

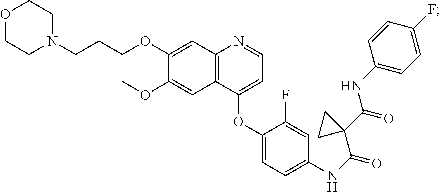 [SMILES: COc1cc2c(Oc3ccc(NC(=O)C4(C(=O)Nc5ccc(F)cc5)CC4)cc3F)ccnc2cc1OCCCN1CCOCC1]

lestaurtinib (or (5S,6S,8R)-6-Hydroxy-6-(hydroxymethyl)-5-methyl-7,8,14,15-tetrahydro-5H-16-oxa-4b,8a,14-triaza-5,8-methanodibenzo[b,h]cycloocta[jkl]cyclopenta[e]-as-indacen-13(6H)-one):

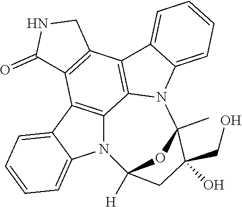 [SMILES: C[C@]12O[C@H](C[C@]1(O)CO)n1c3ccccc3c3c4c(c5c6ccccc6n2c5c31)CNC4=O]

CHEMBL1971029 (or 2-((2-((5-(4-acetylpiperazin-1-yl)-2-methoxyphenyl)amino)-5-bromopyrimidin-4-yl)amino)-N-methylbenzene sulfonamide):

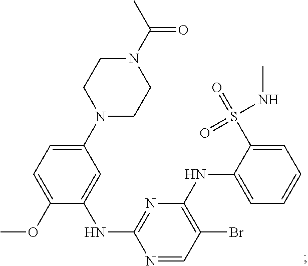 [SMILES: CNS(=O)(=O)c1ccccc1Nc1nc(Nc2cc(N3CCN(C(C)=O)CC3)ccc2OC)ncc1Br]

CHEMBL1241473 (or 2-((5-nitro-2-((3,4,5-trimethoxyphenyl)amino)pyrimidin-4-yl)amino) benzamide:

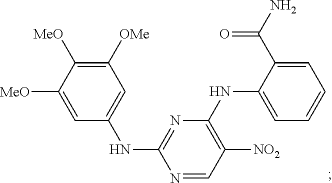 [SMILES: COc1cc(Nc2ncc([N+](=O)[O-])c(Nc3ccccc3C(N)=O)n2)cc(OC)c1OC]

CHEMBL1987034 (or 3-((5-bromo-4-((4-(cyanomethoxy)phenyl)amino)pyrimidin-2-yl)amino) benzenesulfonamide):

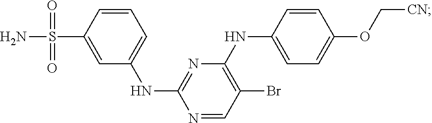 [SMILES: N#CCOc1ccc(Nc2nc(Nc3cccc(S(N)(=O)=O)c3)ncc2Br)cc1]

CHEMBL411903 (or 1-(4-(4-amino-8,9-dihydro-7H-pyrimido[4,5-b][1,4]diazepin-6-yl)phenyl)-3-(2-fluoro-5-(trifluoromethyl)phenyl)urea):

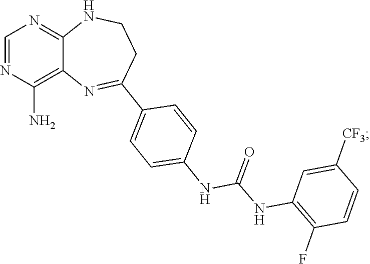 [SMILES: Nc1ncnc2c1N=C(c1ccc(NC(=O)Nc3cc(C(F)(F)F)ccc3F)cc1)CCN2]

CHEMBL2005886 (or 4-((2-(3-chlorophenyl)-2-hydroxyethyl)amino)-3-(7-methyl-5-morpholino-1H-benzo[d]imidazol-2-yl)pyridin-2(1H)-one):

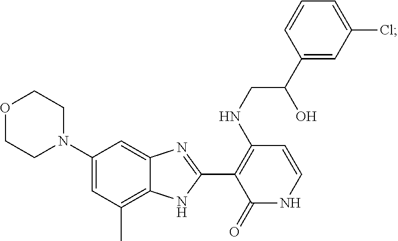 [SMILES: Cc1cc(N2CCOCC2)cc2nc(-c3c(NCC(O)c4cccc(Cl)c4)cc[nH]c3=O)[nH]c12]

CHEMBL1988387 (or 1-(4-(4-amino-7-(1-methyl-1H-pyrazol-4-yl)thieno[3,2-c]pyridin-3-yl)phenyl)-3-(4-(difluoromethoxy)phenyl)urea):

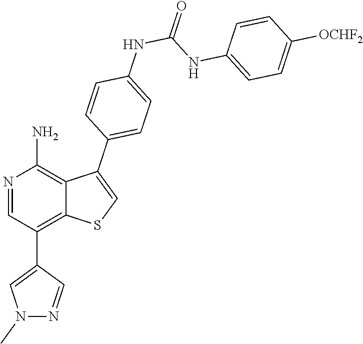 [SMILES: Cn1cc(-c2cnc(N)c3c(-c4ccc(NC(=O)Nc5ccc(OC(F)F)cc5)cc4)csc23)cn1]

CHEMBL1967878 (or 3-((5-fluoro-2-((3-methyl-4-(4-methylpiperazin-1-yl)phenyl)amino) pyrimidin-4-yl)amino)bicyclo[2.2.1]hept-5-ene-2-carboxamide:

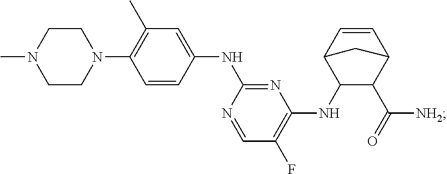 [SMILES: Cc1cc(Nc2ncc(F)c(NC3C4C=CC(C4)C3C(N)=O)n2)ccc1N1CCN(C)CC1]

and
CHEMBL1988838 (or 2-(4-((5-methyl-2-((3-sulfamoylphenyl)amino)pyrimidin-4-yl)amino) phenoxy)acetamide):

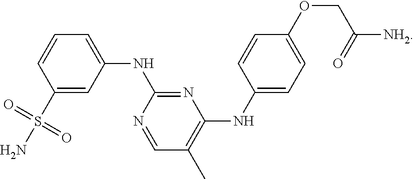 [SMILES: Cc1cnc(Nc2cccc(S(N)(=O)=O)c2)nc1Nc1ccc(OCC(N)=O)cc1]

In certain embodiments, the composition comprises a Pyk2 expression inhibitor. For example, in certain embodiments, the composition comprises an isolated nucleic acid (e.g., siRNA, ribozyme, antisense RNA, etc.) that reduces the expression level of Pyk2 in a cell.

In certain embodiments, small molecules or peptidomimetics contemplated herein are prepared as prodrugs. A prodrug is an agent converted into the parent drug in vivo. In one embodiment, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound. Prodrugs are known to those skilled in the art, and may be prepared using methodology described in the art.

The small molecule and small molecule compounds described herein may be present as salts even if salts are not depicted and it is understood that the invention embraces all salts, prodrugs and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted.

The invention also includes any or all of the stereochemical forms, including any enantiomeric or diastereoisomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In certain embodiments, the small molecule inhibitor of the invention comprises an analog or derivative of an inhibitor described herein. In other embodiments, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, the isotope comprises deuterium. In certain embodiments, isotopically labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In certain embodiments, the pharmaceutical composition is coformulated with at least one additional agent that treats or prevents an Aβ-modulated disease in a mammal, and/or that improves or prevents further loss of cognition in a mammal.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Methods

In certain aspects, the present invention provides a method for treating or preventing an Aβ-modulated disease in a subject. In certain embodiments, the method comprises administering to the subject an effective amount of a Pyk2 inhibitor. In other instances, administration of a Pyk2 inhibitor reduces pathological Aβ mediated signaling and/or reduces the progression of an Aβ-modulated disease.

In certain aspects, the present invention provides a method for improving or preventing further loss of cognition in a subject. In certain embodiments, the method comprises administering to the subject an effective amount of a Pyk2 inhibitor. In other embodiments, administration of a Pyk2 inhibitor restores or prevents further loss of synapse density in the subject.

In certain embodiments, the Aβ-modulated disease or disorder is selected from the group consisting of Alzheimer's Disease (AD), prodromal Alzheimer's Disease, amnestic mild cognitive impairment (MCI), Down syndrome dementia, traumatic brain injury, and frontotemporal dementia.

In certain embodiments, Aβ oligomer-induced signaling is inhibited in the mammal.

In certain embodiments, the Pyk2 inhibitor is selected from the group consisting of a nucleic acid, siRNA, antisense nucleic acid, ribozyme, peptide, antibody, small molecule, antagonist, aptamer, peptidomimetic, and any combinations thereof.

In certain embodiments, the compound and/or composition is administered to the mammal by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes.

In certain embodiments, the mammal is further administered a Fyn inhibitor. In other embodiments, the Pyk2 inhibitor further inhibits Fyn.

In certain embodiments, the method further comprises administering to the mammal at least one additional agent that treats or prevents the Aβ-modulated disease or disorder in the mammal. In other embodiments, the composition and at least one additional agent are coformulated. In yet other embodiments, the mammal is human.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from ng/kg/day and 100 mg/kg/day. In certain embodiments, the invention envisions administration of a dose which results in a concentration of the compound of the present invention from 1 μM and 10 μM in a mammal. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the invention.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., anti-AD agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

In one embodiment, the tablets of the invention comprise a compound contemplated within the invention, mannitol, dibasic calcium phosphate anhydrous, crospovidone, hypromellose and magnesium stearate, with a film-coat containing hypromellose, macrogol 400, red iron oxide, black iron oxide and titanium dioxide. In other embodiments, the tablets of the invention comprise the compound expressed as free base or a salt thereof.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized.

### Combination Therapies

In certain embodiments, the compounds of the invention are useful in the methods of the invention in combination with at least one additional agent useful for treating or preventing an Aβ-modulated disease, and/or improving or preventing further loss in cognition, in a mammal in need thereof. This additional agent may comprise compounds identified herein or compounds, e.g., commercially available compounds, known to treat, prevent or reduce the symptoms of an Aβ-modulated disease in a subject.

In certain embodiments, the at least one additional compound useful for treating or preventing an Aβ-modulated disease comprises acetylcholinesterase inhibitors, such as, but not limited to donepezil ((RS)-2-[(1-benzyl-4-piperidyl)methyl]-5,6-dimethoxy-2,3-dihydroinden-1-one) or memantine (3,5-dimethyladamantan-1-amine). In other embodiments, the at least one additional compound useful for treating or preventing an Aβ-modulated disease comprises an anti-amyloid agent, such as an antibody or a small molecule.

In certain embodiments, the at least one additional compound useful for improving or preventing further loss in cognition comprises a drug approved for treating Alzheimer's Disease, such as acetylcholinesterase inhibitors (such as donepezil) or memantine.

A synergistic effect may be calculated, for example, using suitable methods such as, for example, the Sigmoid-Emax equation (Holford & Scheiner, 1981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Kits

The invention includes a kit comprising at least one Pyk2 inhibitor, an applicator, and an instructional material for use thereof. The instructional material included in the kit comprises instructions for preventing or treating an Aβ-modulated disease in a mammal. The instructional material recites the amount of, and frequency with which, the Pyk2 inhibitor should be administered to the mammal. In other embodiments, the kit further comprises at least one additional agent that prevents or treats an Aβ-modulated disease in a mammal. In other embodiments, the kit further comprises at least one additional agent that improves and/or prevent further loss of cognition in a mammal.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. As used herein, each of the following terms has the meaning associated with it in this section.

Generally, the nomenclature used herein and the laboratory procedures in cell culture, molecular genetics, pharmacology and organic chemistry are those well-known and commonly employed in the art.

Standard techniques are used for biochemical and/or biological manipulations. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The following abbreviations are used herein: Aβ—amyloid-β; Aβo—Aβ oligomers; AD—Alzheimer's Disease; AZD0530 or AZD-0530—saracatinib or N-(5-chloro-1,3-benzodioxol-4-yl)-7-[2-(4-methyl-1-piperazinyl)ethoxy]-5-[(tetrahydro-2H-pyran-4-yl)oxy]-4-quinazolinamine or a salt or solvate thereof; CSF—cerebrospinal fluid; EEG—electroencephalogram; fEPSP—field excitatory postsynaptic potential; LOAD—late-onset AD; MCI—mild cognitive impairment; NAM—negative allosteric modulator; NMDA—N-methyl D aspartate; NMDA-R—NMDA receptor; PET—positron emission tomography; PrPC—prion protein; PSD—post-synaptic density; SAM—senescence accelerated mice; SEM—standard error of mean; TBI—traumatic brain injury; TBS—theta burst stimulation.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, an “Aβ-modulated disease” or “Aβ-modulated disorder” refers to a neurological disease that is associated with pathological Aβ accumulation or Aβ-mediating signaling. Non-limiting examples of such diseases encompass, but are not limited to, Alzheimer's Disease (AD), prodromal Alzheimer's Disease, amnestic mild cognitive impairment (MCI), Down syndrome dementia, traumatic brain injury, Lewy body dementia, Parkinson's Disease with dementia, frontotemporal dementia, and after stroke aphasia.

As used herein, the term “AD” refers to Alzheimer's Disease.

A disease or disorder is “alleviated” if the severity or frequency of at least one sign or symptom of the disease or disorder experienced by a patient is reduced.

As used herein, the terms “analog,” “analogue,” or “derivative” are meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate. A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Naturally occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” or “therapeutically acceptable salt” refers to a salt of the administered compounds prepared from pharmaceutically acceptable non-toxic acids, including inorganic acids or bases, organic acids or bases, solvates, hydrates, or clathrates thereof.

The terms “pharmaceutically effective amount” and “effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, the terms “polypeptide,” “protein” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The phrase “Pyk2 inhibitor,” or “inhibitor of Pyk2” as used herein, refers to a composition or compound that inhibits at least in part, as compared to the control system that lacks the inhibitor, Pyk2 activity, Pyk2 expression and/or both, either directly or indirectly, using any method known to the skilled artisan. A Pyk2 inhibitor may be any type of compound, including but not limited to, a nucleic acid, peptide, antibody, small molecule, antagonist, aptamer, or peptidomimetic.

By the term “specifically binds,” as used herein, is meant a molecule, such as an antibody, which recognizes and binds to another molecule or feature, but does not substantially recognize or bind other molecules or features in a sample.

A “therapeutic” treatment is a treatment administered to a subject who exhibits signs of pathology, for the purpose of diminishing or eliminating those signs.

As used herein, the term “therapeutically effective amount” is an amount of a compound of the invention, that when administered to a patient, ameliorates a symptom of the disease or disorder. The amount of a compound of the invention that constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms. As used herein, the term “wild-type” refers to the genotype and phenotype that is characteristic of most of the members of a species occurring naturally and contrasting with the genotype and phenotype of a mutant.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Methods and Materials

### PYK2 Kinase Assay

ADP-Glo™ (Promega, madison, WI) Kinase Assay is a luminescent kinase assay that measures ADP formed from a kinase reaction; ADP is converted into ATP, which is converted into light by Ultra-Glo™ Luciferase. The luminescent signal positively correlates with ADP amount and kinase activity. The assay is well suited for measuring the effects chemical compounds have on the activity of a broad range of purified kinases making it ideal for both primary screening as well as kinase selectivity profiling. The ADP-Glo™ Kinase Assay can be used to monitor the activity of virtually any ADP-generating enzyme (e.g., kinase or ATPase) using up to 1 mM ATP.

Protocol:

Enzyme, substrate, ATP and inhibitors are diluted in Tyrosine Kinase Buffer. 1 μl of inhibitor or (5% DMSO), 2 μl of enzyme, and 2 μl of substrate/ATP mix are added to the wells of 384 low volume plate. The resulting system is incubated at room temperature for 60 minutes. 5 μl of ADP-GLO™ Reagent are added. The resulting system is incubated at room temperature for 40 minutes. 10 μl of Kinase Detection Reagent are added. The resulting system is incubated at room temperature for 30 minutes. Luminescence is recorded (Integration time 0.5-1 second).

### FYN A Kinase Assay

ADP-GLO™ (Promega, madison, WI) Kinase Assay is a luminescent kinase assay that measures ADP formed from a kinase reaction; ADP is converted into ATP, which is converted into light by ULTRA-GLO™ Luciferase. The luminescent signal positively correlates with ADP amount and kinase activity. The assay is well suited for measuring the effects chemical compounds have on the activity of a broad range of purified kinases making it ideal for both primary screening as well as kinase selectivity profiling. The ADP-GLO™ Kinase Assay can be used to monitor the activity of virtually any ADP-generating enzyme (e.g., kinase or ATPase) using up to 1 mM ATP.

Protocol:

Enzyme, substrate, ATP and inhibitors are diluted in Kinase Buffer. 1 μl of inhibitor (5% DMSO), 2 μl of enzyme, and 2 μl of substrate/ATP mix are added to the wells of 384 low volume plate. The system is incubated at room temperature for 60 minutes. 5 μl of ADP-Glom′ Reagent are added, and the resulting system is incubated at room temperature for 40 minutes. 10 μl of Kinase Detection Reagent are added, and the resulting system is incubated at room temperature for 30 minutes. Luminescence is recorded (Integration time 0.5-1 second).

### Example 1: Assess Genetic Necessity for Pyk2 in AD Related Phenotypes

To test the hypothesis that Pyk2 is essential for manifestations of familial AD genes in mice, Pyk2−/− mice were bred with APPswe/PS1ΔE9 mice and disease progression without Pyk2 was assessed.

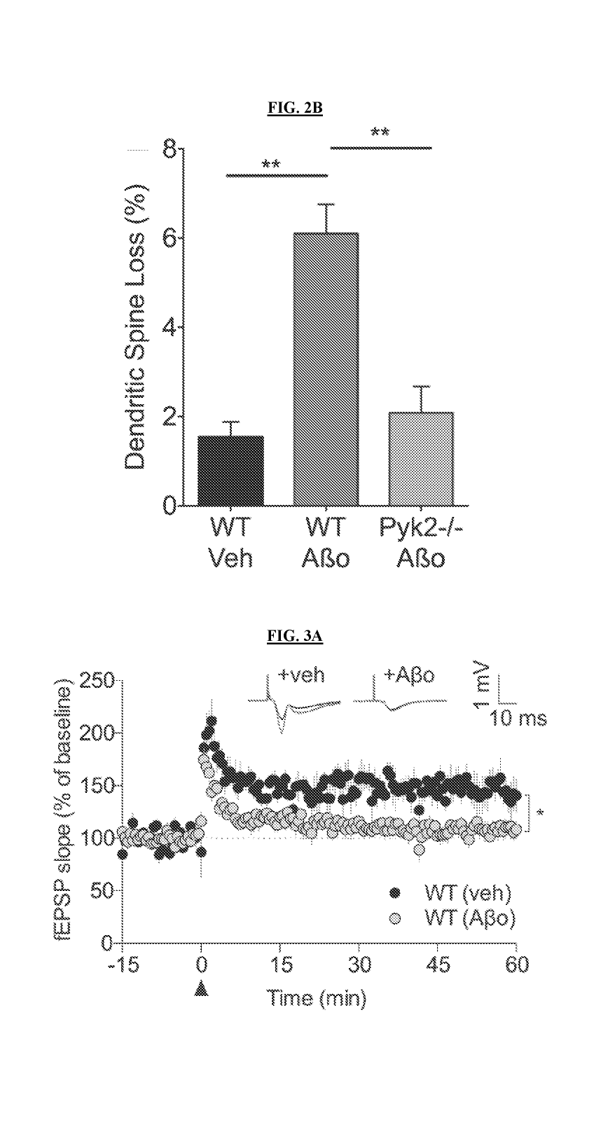

If Pyk2 is coupled to AD signaling, then it is predicted to associate with Aβo receptors. Pyk2 co-immunoprecipitates with PrPC in mouse and human brain tissue. mGluR5 is an essential link between them. Moreover, the association of Pyk2 with the PrPC/mGluR5 complex is regulated by Aβo in mouse and human brain. Further, Aβo-treated slices or AD transgenic brain show activated Pyk2, and that activation is mediated by Prnp-Grm5 interaction. In a synaptic assay, exposure to Aβo induces dendritic spine loss over 6 hours in WT neurons. Dendritic spines of Pyk2 null neurons are fully protected from Aβo (FIGS. 2A-2B).

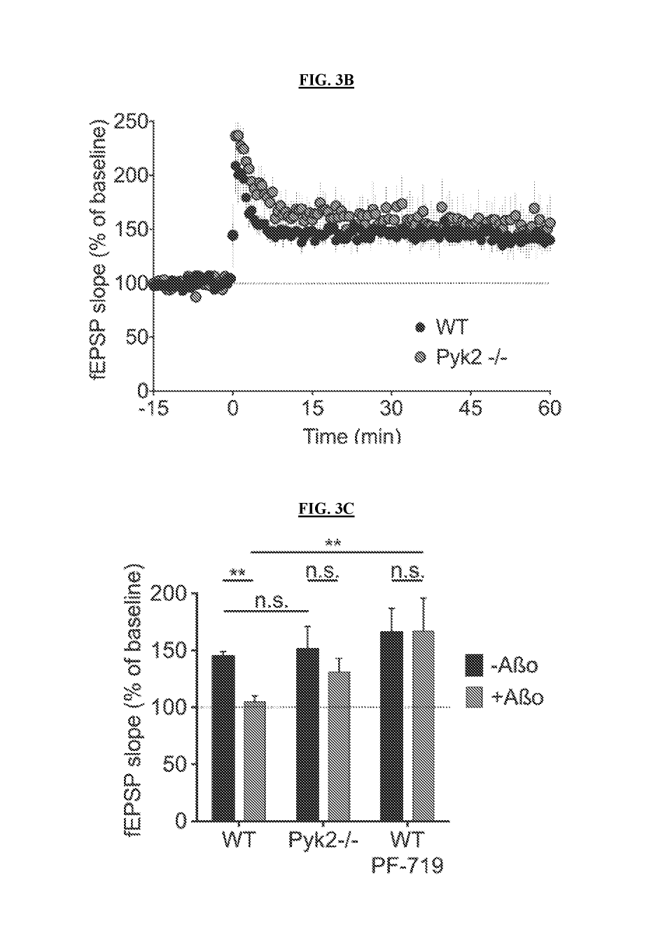

The role of Pyk2 in Aβo-induced disruption of synaptic plasticity using hippocampal CA3 to CA1 LTP has also been evaluated, comparing WT and Pyk2−/− slices. Recordings utilizing Pyk2 null slices have been completed and also with nM concentrations of the Pyk2 inhibitor, PF-719. The magnitude of LTP in slices without Pyk2 is as robust as for WT slices (FIGS. 3B-3C). Acute Pyk2 kinase inhibition does not block LTP (FIG. 3C). Most critically, slices have been treated with Aβo (FIG. 3A), and either Pyk2 deletion or Pyk2 inhibition protects these slices from Aβo-induced suppression (FIG. 3C).

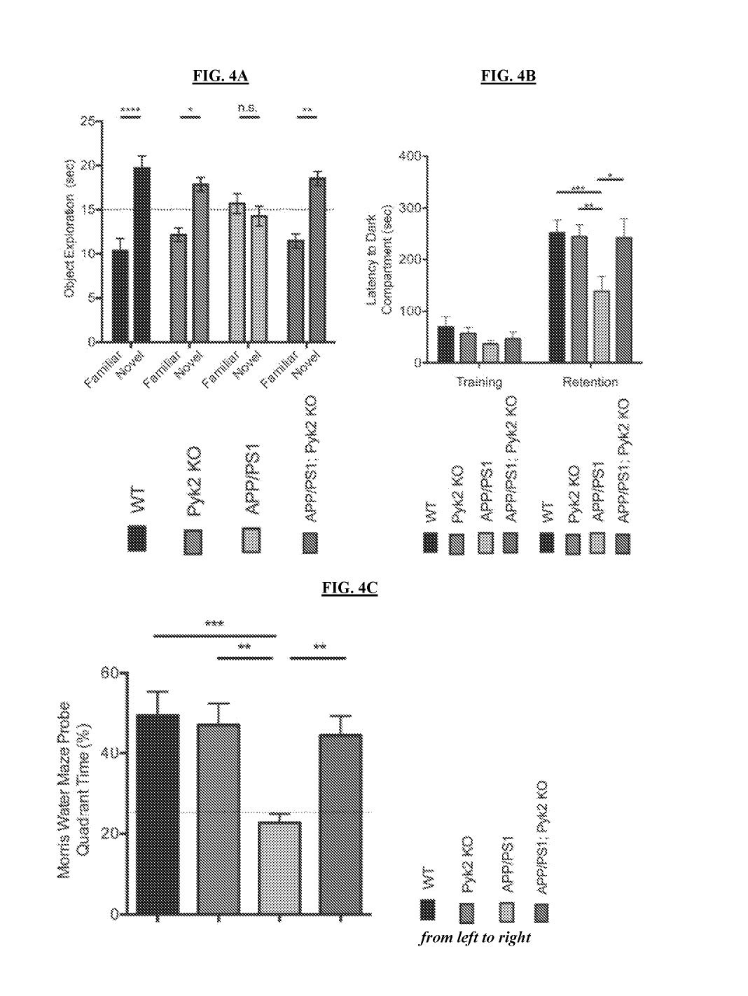

Based on the data that Pyk2 mediates Aβo signaling, dendritic spine loss and synaptic electrophysiology, APP/PS1 mice on the Pyk2−/− C57Bl6J background were studied. Four genotypes, WT, Pyk2−/−, APP/PS1, and APP/PS1, Pyk2−/−, were bred and aged to 12 months for memory testing. Three memory tests were assessed: novel object recognition, passive avoidance learning and Morris water maze. In each paradigm, the aged APP/PS1 have a pronounced deficit (FIGS. 4A-4C). For control mice without AD pathology, the deletion of Pyk2 did not alter memory function in any of the three tests. However, the APP/PS1 mice on the Pyk2 null background were indistinguishable from the WT mice in all three tests. The brains of these mice are analyzed for synapse density, gliosis and Aβ. Pyk2 is thus required for the learning and memory phenotypes induced by APP/PS1 transgenes in mice.

### Example 2: Evaluating Pharmacological Inhibition of Pyk2 in Suppressing AD Phenotypes

Evidence for brain accumulation of the drug was obtained at Pyk2 inhibitor concentrations required to inhibit Pyk2 after peripheral dosing. Therefore, the ability of PF-719 inhibitor to reverse deficits in AD transgenic mice was tested.

PF-719 has a Kᵢ of 15 nM for isolated Pyk2 versus 450 nM for FAK (30× selectivity). In brain slices and in dissociated primary neuronal cultures, Aβo causes Fyn activation, Pyk2 activation, altered Glu-induced calcium signaling, dendritic spine loss and suppression of LTP. PF-719 (500 nM) blocked baseline Pyk2 activation and Aβo-induced signaling in cells (FIG. 6C). After developing LC/MS/MS methods to detect PF-719, the extent to which the Pyk2 inhibitor enters brain from peripheral dosing was determined. After a single peripheral dose of 5 mg/kg i.p. or p.o., brain levels are 450±110 nM at 90 min, about 30% of plasma level with a half-life of 6 hours. This level is 30 fold above the Kᵢ, and equal to the concentration that suppresses p-Pyk2 in tissue culture. Continuous 25 mg/kg/d minipump i.p. infusion was chosen for treatment of APP/PS1 mice. Oral bioavailability is equal but, in mouse, oral gavage every 6-8 hours is impractical over 4-6 weeks.

The efficacy of PF-719 Pyk2 inhibitor to rescue AD transgenic phenotypes was tested by infusion for 6 weeks in 12 month APP/PS1 transgenic with pre-established memory deficits and WT mice, similar to the SAM 923 and Pyk2−/− experiments. In these experiments, the vehicle-treated and untreated APP/PS1 mice again showed severe impairments in novel object recognition, and spatial memory during the probe phase of the Morris water maze (FIGS. 5A-5B). For APP/PS1 mice treated with PF-719 peripherally for 3-6 weeks, learning and memory performance were significantly greater than Vehicle/untreated levels (FIGS. 5A-5B). The brains of these mice are analyzed for synapse density, gliosis and Aβ. Pyk2 inhibition thus reverses the learning and memory phenotypes induced by APP/PS1 transgenes in mice.

### Example 3: Demonstration of Dual Inhibitory Activities

An in silico screen of the CHEMBL & IUPHR-BPS database uncovered 12 compounds with Kᵢ's less than 100 nm for both Pyk2 and Fyn.

TABLE 2
Exemplary Kinase Inhibitors

| | | | Inhibition |
|---|---|---|---|
| Compound | Pyk2 Ki (nM) | Fyn Ki (nM) | Character |
| PF-719 | 10 | >1,000 | Pyk2 selective |
| AZD-0530 | >1,000 | 5 | Fyn selective |
| Tamatinib | 29 | 28 | dual Pyk2-Fyn |
| Foretinib | 14 | 88 | dual Pyk2-Fyn |
| Lestaurtinib | 41 | 84 | dual Pyk2-Fyn |
| Compound 1 | 2 | 25 | dual Pyk2-Fyn |
| Compound 2 | 8 | 8 | dual Pyk2-Fyn |
| Compound 3 | 10 | 25 | dual Pyk2-Fyn |
| Compound 4 | 20 | 79 | dual Pyk2-Fyn |
| Compound 5 | 25 | 25 | dual Pyk2-Fyn |
| Compound 6 | 63 | 6 | dual Pyk2-Fyn |
| Compound 7 | 79 | 8 | dual Pyk2-Fyn |
| Compound 8 | 100 | 63 | dual Pyk2-Fyn |

PF-719 (or 4-(((6-ethoxybenzo[d]thiazol-2-yl)thio)methyl)-6-morpholino-1,3,5-triazin-2-amine):

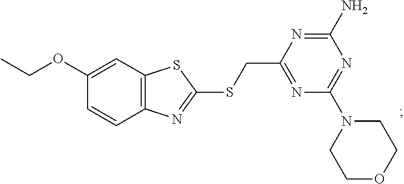 [SMILES: CCOc1ccc2nc(SCc3nc(N)nc(N4CCOCC4)n3)sc2c1]

AZD-0530 (or N-(5-chloro-1,3-benzodioxol-4-yl)-7-[2-(4-methyl-1-piperazinyl)ethoxy]-5-[(tetrahydro-2H-pyran-4-yl)oxy]-4-quinazolinamine):

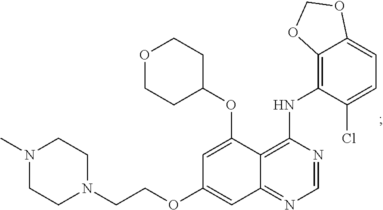 [SMILES: CN1CCN(CCOc2cc(OC3CCOCC3)c3c(Nc4c(Cl)ccc5c4OCO5)ncnc3c2)CC1]

tamatanib (or 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one):

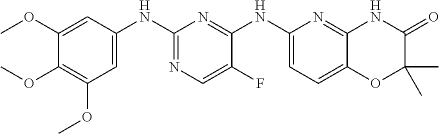

foretinib (or N-(3-fluoro-4-((6-methoxy-7-(3-morpholinopropoxy)quinolin-4-yl)oxy)phenyl)-N-(4-fluorophenyl)cyclopropane-1,1-dicarboxamide):

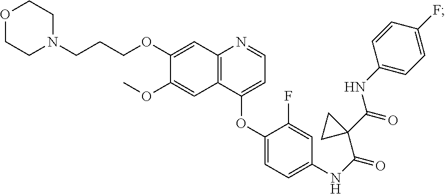 [SMILES: COc1cc2c(Oc3ccc(NC(=O)C4(C(=O)Nc5ccc(F)cc5)CC4)cc3F)ccnc2cc1OCCCN1CCOCC1]

lestaurtinib (or (5S,6S,8R)-6-Hydroxy-6-(hydroxymethyl)-5-methyl-7,8,14,15-tetrahydro-5H-16-oxa-4b,8a,14-triaza-5,8-methanodibenzo[b,h]cycloocta[jkl]cyclopenta[e]-as-indacen-13(6H)-one):

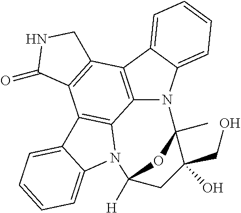 [SMILES: C[C@]12O[C@H](C[C@]1(O)CO)n1c3ccccc3c3c4c(c5c6ccccc6n2c5c31)CNC4=O]

Compound 1 (CHEMBL1971029, or 2-((2-((5-(4-acetylpiperazin-1-yl)-2-methoxyphenyl)amino)-5-bromopyrimidin-4-yl)amino)-N-methylbenzene sulfonamide):

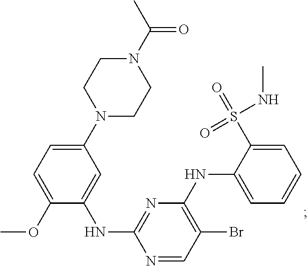 [SMILES: CNS(=O)(=O)c1ccccc1Nc1nc(Nc2cc(N3CCN(C(C)=O)CC3)ccc2OC)ncc1Br]

Compound 2 (CHEMBL1241473, or 2-((5-nitro-2-((3,4,5-trimethoxyphenyl)amino)pyrimidin-4-yl)amino) benzamide:

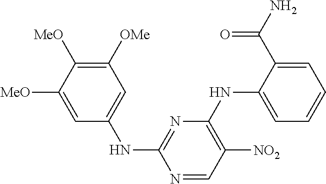 [SMILES: COc1cc(Nc2ncc([N+](=O)[O-])c(Nc3ccccc3C(N)=O)n2)cc(OC)c1OC]

Compound 3 (CHEMBL1987034, or 3-((5-bromo-4-((4-(cyanomethoxy)phenyl)amino) pyrimidin-2-yl)amino) benzenesulfonamide):

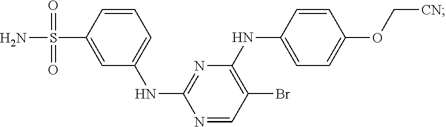 [SMILES: N#CCOc1ccc(Nc2nc(Nc3cccc(S(N)(=O)=O)c3)ncc2Br)cc1]

Compound 4 (CHEMBL411903, or 1-(4-(4-amino-8,9-dihydro-7H-pyrimido[4,5-b][1,4]diazepin-6-yl)phenyl)-3-(2-fluoro-5-(trifluoromethyl)phenyl)urea):

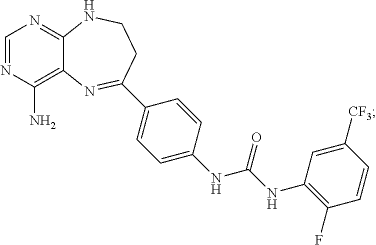 [SMILES: Nc1ncnc2c1N=C(c1ccc(NC(=O)Nc3cc(C(F)(F)F)ccc3F)cc1)CCN2]

Compound 5 (CHEMBL2005886, or 4-((2-(3-chlorophenyl)-2-hydroxyethyl)amino)-3-(7-methyl-5-morpholino-1H-benzo[d]imidazol-2-yl)pyridin-2(1H)-one):

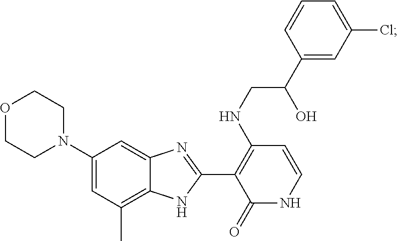 [SMILES: Cc1cc(N2CCOCC2)cc2nc(-c3c(NCC(O)c4cccc(Cl)c4)cc[nH]c3=O)[nH]c12]

Compound 6 (CHEMBL1988387, or 1-(4-(4-amino-7-(1-methyl-1H-pyrazol-4-yl)thieno[3,2-c]pyridin-3-yl)phenyl)-3-(4-(difluoromethoxy)phenyl)urea):

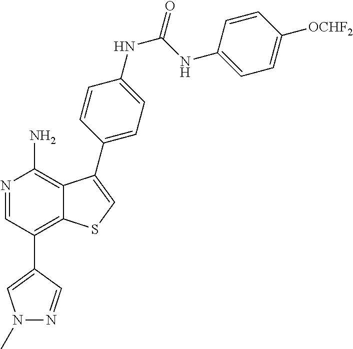 [SMILES: Cn1cc(-c2cnc(N)c3c(-c4ccc(NC(=O)Nc5ccc(OC(F)F)cc5)cc4)csc23)cn1]

Compound 7 (CHEMBL1967878, or 3-((5-fluoro-2-((3-methyl-4-(4-methylpiperazin-1-yl)phenyl)amino) pyrimidin-4-yl)amino)bicyclo[2.2.1]hept-5-ene-2-carboxamide:

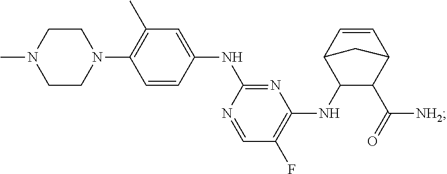 [SMILES: Cc1cc(Nc2ncc(F)c(NC3C4C=CC(C4)C3C(N)=O)n2)ccc1N1CCN(C)CC1]

and
Compound 8 (CHEMBL1988838, or 2-(4-((5-methyl-2-((3-sulfamoylphenyl)amino)pyrimidin-4-yl)amino) phenoxy)acetamide):

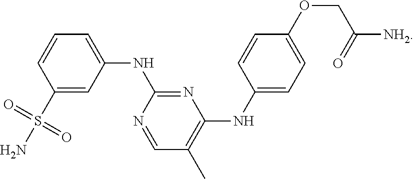 [SMILES: Cc1cnc(Nc2cccc(S(N)(=O)=O)c2)nc1Nc1ccc(OCC(N)=O)cc1]

Luciferase-based biochemical assays of Fyn and Pyk2 were established utilizing commercially available Fyn and Pyk2 enzyme, substrate and luciferase. These assays were used to confirm the respective specificities of Fyn (saracatinib, AZD0530) and Pyk2 (pf-719) inhibitors, as well as a small molecule (tamatinib) from 12 identified in the CHEMBL database as inhibiting both Fyn and Pyk2 (FIGS. 6A-6B).

### Example 4: AD Risk Factor Pyk2 Mediates Amyloid-β Induced Synaptic Dysfunction and Loss Through Interaction with Graf1 and Rho Activation

As demonstrated herein, the risk factor Pyk2 (also known as PTK2B) is activated in human AD brain tissue and localizes specifically to neurons in post-synaptic regions. Pyk2 overexpression reduces dendritic spine density of cortical neurons by a kinase-dependent mechanism. Biochemical isolation of Pyk2-interacting proteins from brain identifies Graf1c, a RhoA GTPase-activating protein inhibited by Pyk2. Aβo-induced reductions in dendritic spine acute motility and chronic loss require both Pyk2 kinase and RhoA activation. Deletion of Pyk2 expression also protects hippocampal slices from Aβo-induced suppression of long term potentiation. Transgenic AD model mice expressing APPswe/PS14E9 require Pyk2 for age-dependent loss of synaptic markers and for impairment of learning and memory. Thus, the Pyk2 risk gene is directly implicated in a neuronal Aβo signaling pathway impairing synaptic anatomy and function.

Selected results are illustrated herein:

### Pyk2 is Activated in Human AD and Induces Spine Loss in Neurons

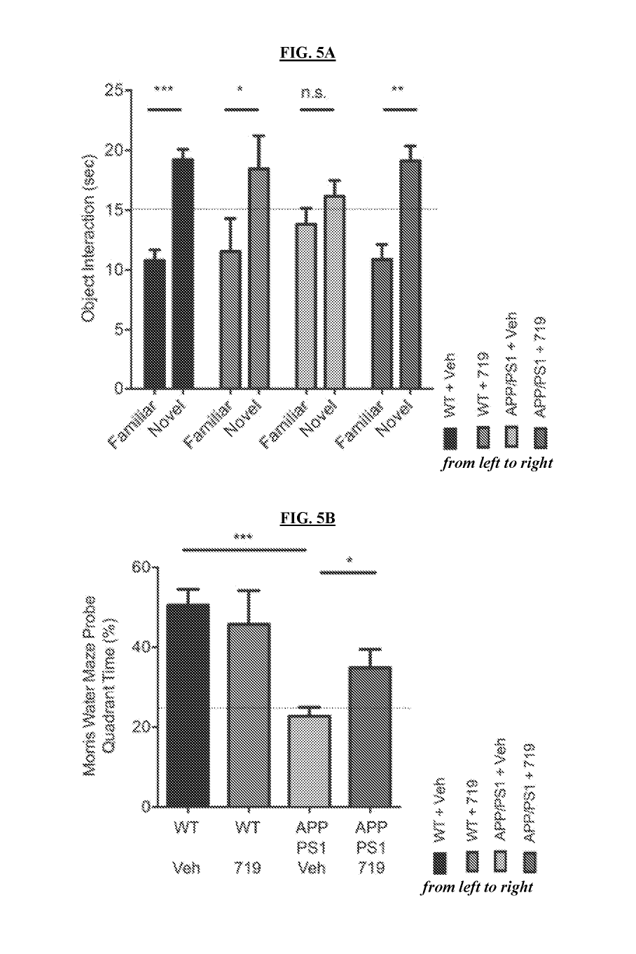

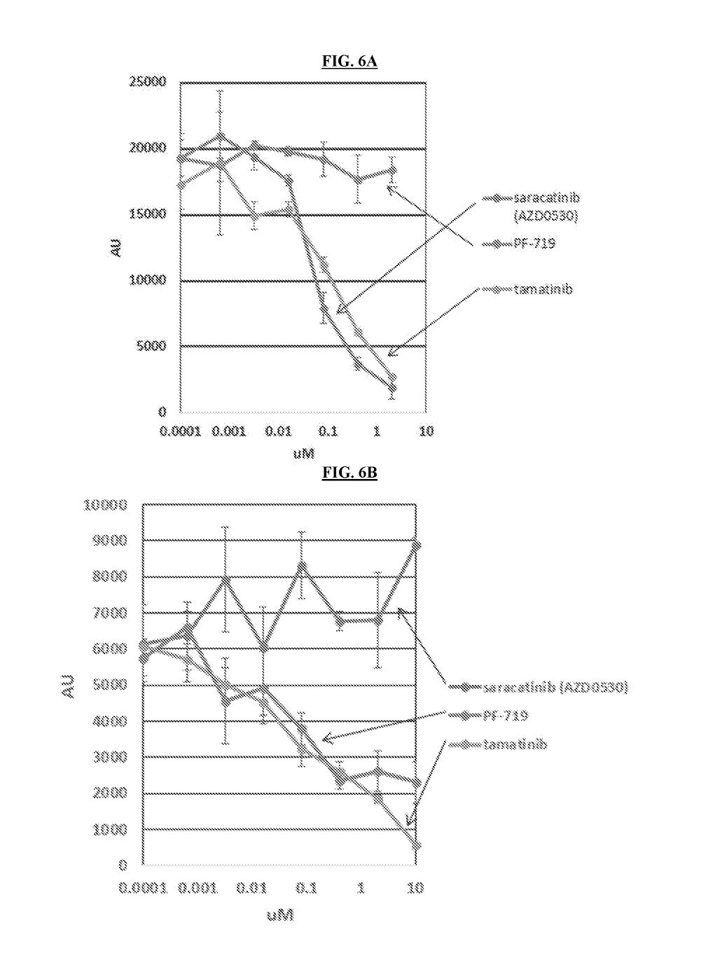

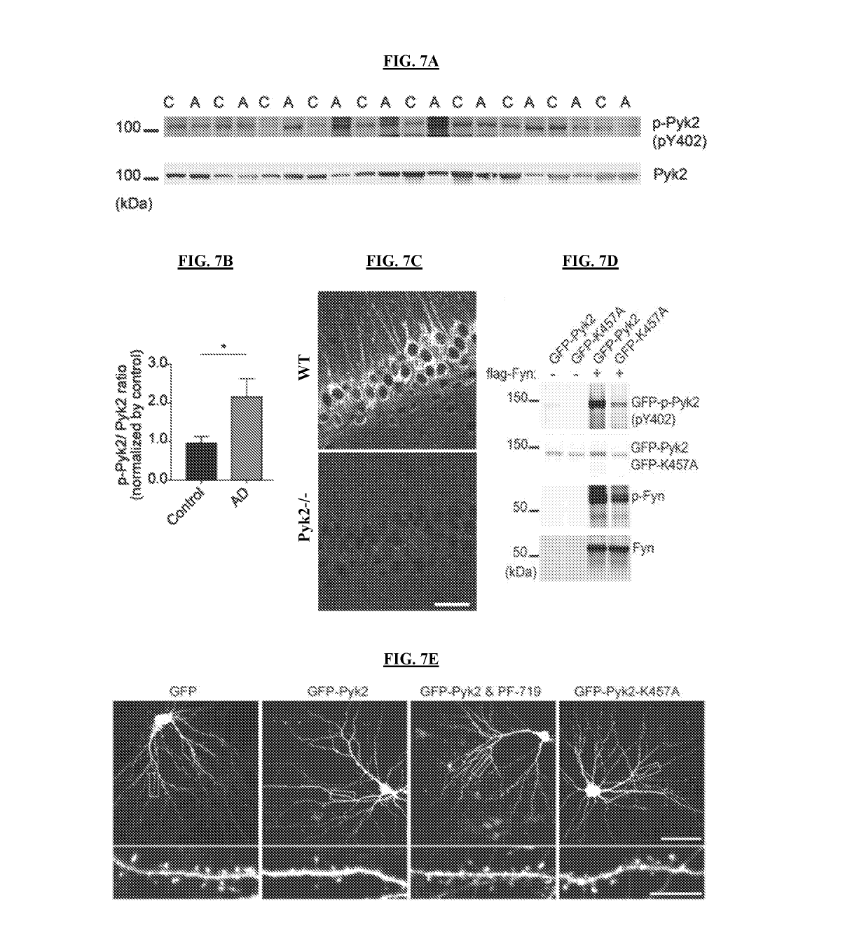

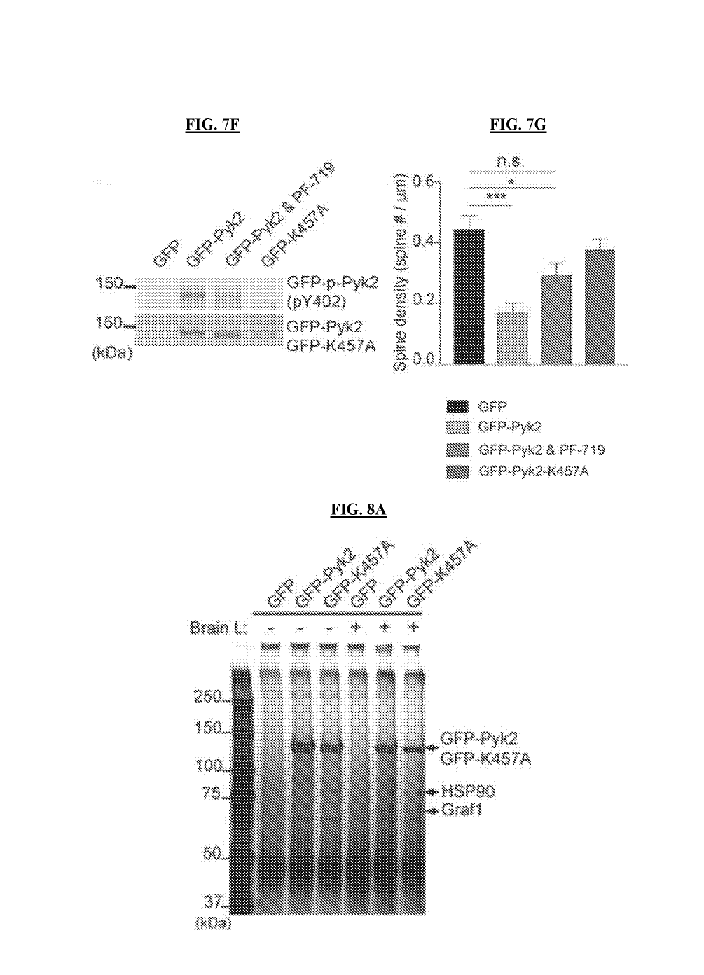

While Pyk2 is a risk factor for LOAD and is activated in transgenic AD mice, the Pyk2 protein level and activation in AD brain is not clear. Pyk2 activation was measured in frontal cortex from human autopsy brain tissue by phosphorylation of Y402, a marker of Pyk2 activation. A previously described collection of brain samples from human control neurologically intact without Aβ plaque (Braak stage 0-II) and AD (Braak stage V and greater with CERAD neuritic plaque score of Frequent) was examined (Kostylev, et al., 2015, J. Biol. Chem. 290:17415-17438). There is a significant increase in Pyk2 phosphorylation in AD detergent soluble extracts compared to samples from neurologically intact, AD pathology negative controls (FIGS. 7A-7B). This correlation suggests increased Pyk2 function might contribute to AD.

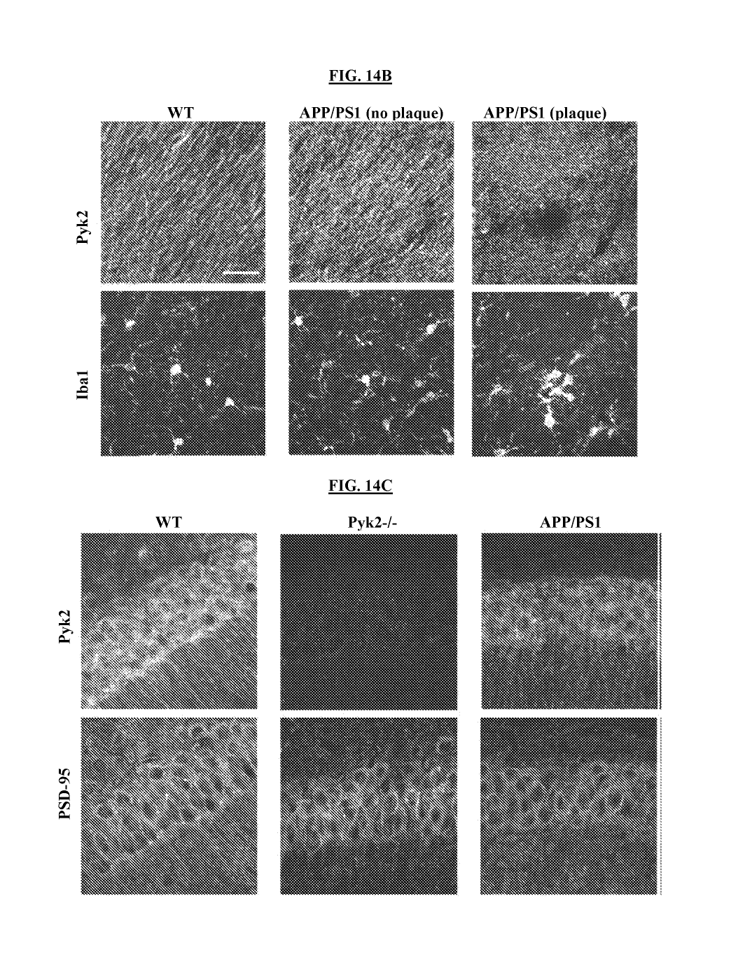

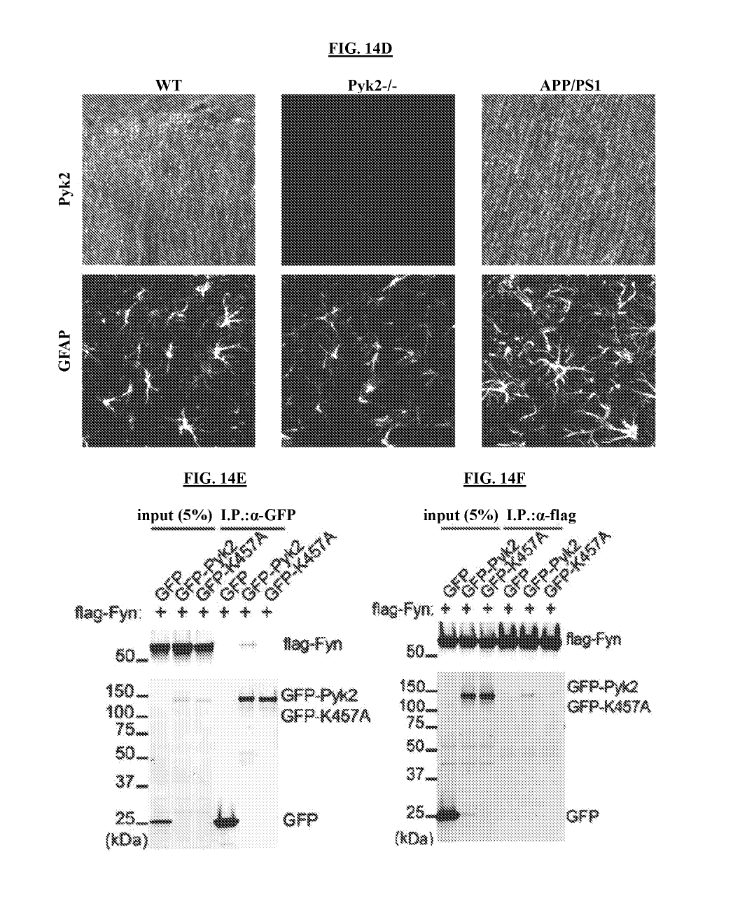

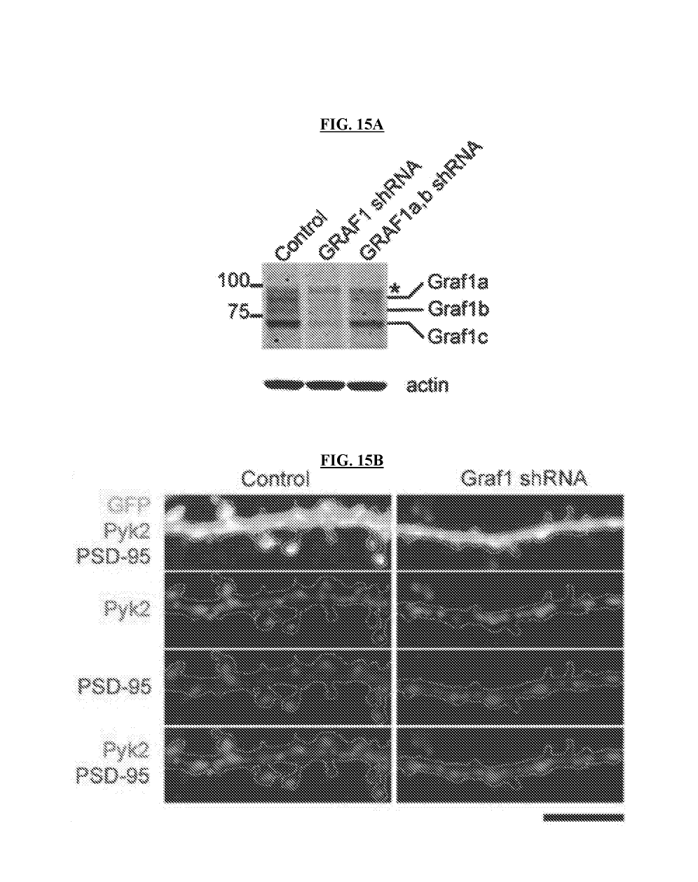

Activated Pyk2 was observed in both human AD and transgenic models, and the cellular localization of Pyk2 was assessed. Pyk2 expression was characterized in mouse brain by immunohistology, with Pyk2−/− tissue for control. The vast majority of Pyk2 was localized to the neuropil (FIG. 7C), and Pyk2 colocalized with MAP2 and PSD95, consistent with a neuronal identity of Pyk2 (FIGS. 14A-14B). Furthermore, little or no Pyk2 was colocalized with the astrocyte marker GFAP, or the microglial marker Iba1 (FIGS. 14C-14D). There was no detectable change in Pyk2 localization in samples from aged transgenic AD mice. Without wishing to be limited by any theory, any Pyk2 role in AD is likely to occur within neurons.

The Fyn-Pyk2 complex was evaluated this complex using an Hek293T overexpression system (FIG. 7D). Co-expression of GFP-Pyk2 with flag-Fyn induced an increase in Pyk2 phosphorylation, but overexpression of a catalytically inactive Pyk2 mutant (GFP-K457A) displayed a robust decrease in both Pyk2 phosphorylation and Fyn phosphorylation. Without wishing to be limited by any theory, these data suggest the kinase domain of Pyk2 is necessary for the majority of synergistic cross-phosphorylation observed in both Pyk2 and Fyn. To further characterize Pyk2-Fyn interaction, immunoprecipitation experiments were performed to determine whether the kinase activity of Pyk2 was necessary for Pyk2-Fyn binding. Immunoprecipitation of GFP-Pyk2 using a GFP antibody was able to co-immunoprecipitate flag-Fyn (FIG. 14E) and similarly, immunoprecipitating flag-Fyn co-immunoprecipitated Pyk2 (FIG. 14F). This interaction was dependent on the Pyk2 kinase activity, as the kinase inactive GFP-K457A mutant failed to interact with flag-Fyn (FIGS. 14E-14F). Thus, Pyk2 interacts with the AD-associated Fyn kinase in a cross-activating manner requiring Pyk2 kinase activity.

To evaluate the consequences of Pyk2 gain-of-function in AD, Pyk2 was overexpressed in primary culture of mouse hippocampal neurons. Overexpressed GFP-Pyk2 is activated by autophosphorylation on Y402 residue and expressed throughout dendrites, and decreases spine density by 60% compared to GFP-transfected neurons (FIGS. 7E-7G). To test whether the kinase activity of Pyk2 was responsible for the observed spine loss, GFP-Pyk2 expressing neurons were pretreated with a specific small molecule inhibitor of Pyk2, PF-719, and partial rescue of GFP-Pyk2 induced neuron loss was observed. Concordantly, overexpressing the kinase inactive GFP-K457A mutant of Pyk2 did not alter dendritic spine density. Thus, increased Pyk2 kinase activity induces synapse loss, consistent with Pyk2 activation contributing to synapse loss in AD.

Pyk2 Interacts with Graf1c and Localizes to Synapses

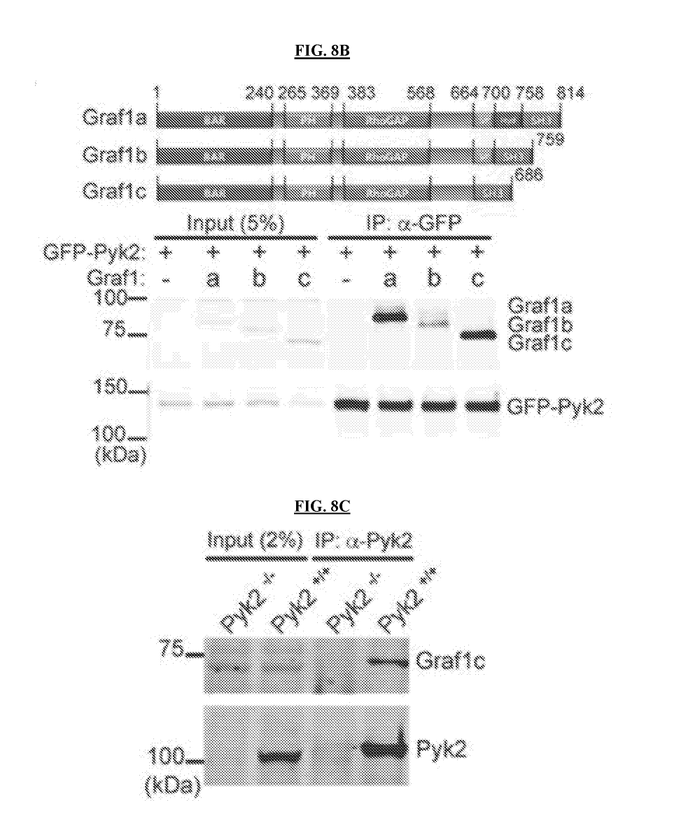

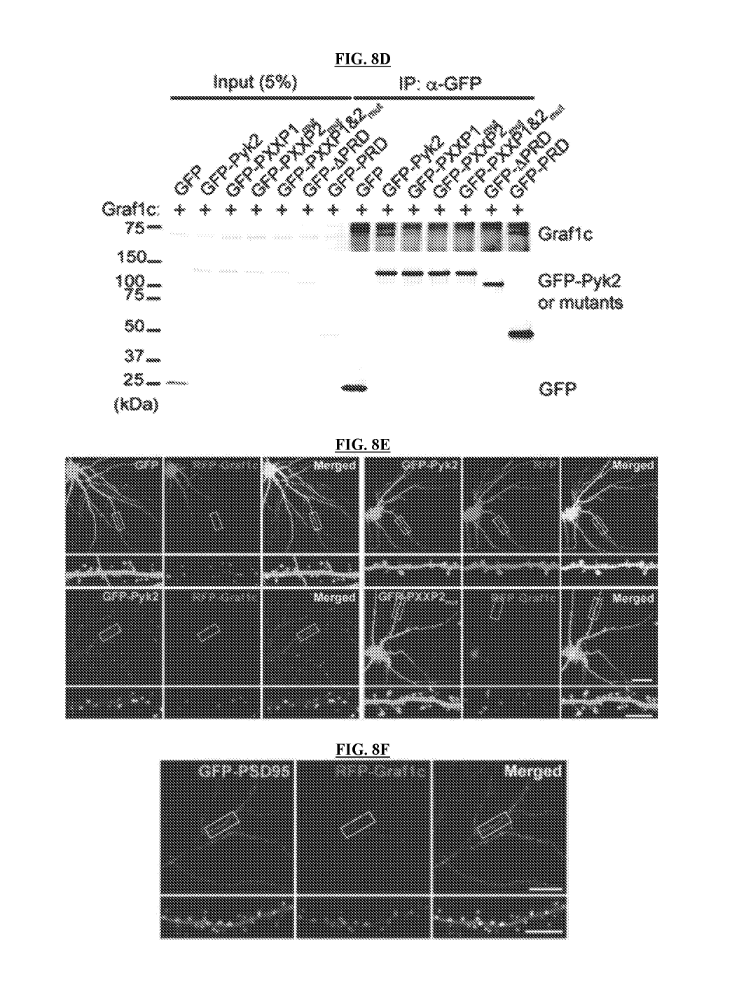

The molecular basis for Pyk2 action was explored: Pyk2 immunoprecipitates were examined for interacting proteins. Lysate was harvested from GFP, GFP-Pyk2, and GFP-K457A expressing Hek293T cells alone, and the same lysates were spiked into WT brain lysate prior to immunoprecipitation with a GFP antibody (FIG. 8A). The two most prominent silver-stained protein species from the brain pull-down other than GFP-Pyk2 were excised and analyzed by LC-MS/MS. HSP90 and Graf1 were identified as Pyk2-intearcting proteins. HSP-90 is present in the Hek cell sample without brain lysate, while Graf1c is detected clearly only when brain lysate is added. HSP-90 interacts only with the mutant Pyk2, while Graf1 interacts with both Pyk2 species. GTPase Regulator Associated with Focal Adhesion Kinase-1 (Graf1) is known to exhibit GAP activity for RhoA and Cdc42, and the Graf1 BAR domain suggests a membrane sculpting function in clathrin-independent endocytosis (FIG. 8B). Given the spine loss data described above (FIGS. 7E-7G), attention was focused on Graf1 and the Pyk2-Graf1 interaction. Graf1 is highly expressed in the heart, brain, and lung and exists in three distinct isoforms. Therefore, it was investigated if Pyk2 has a preference for binding amongst Graf1 isoforms. By immunoprecipitation of GFP-Pyk2 from Hek293T cells, interaction was observed with all three isoforms, but preferentially binding to isoforms a and c (FIG. 8B). Graf1c is the predominant species expressed in WT brain and endogenous protein co-immunoprecipitates with endogenous Pyk2 (FIG. 8C). Since both Pyk2 and Graf1c encode SH3 domains (Proline-rich domain-PRD), it was considered whether the Pyk2-Graf1c interaction is facilitated through the PRD regions of Pyk2. An immunoprecipitation experiment was performed with Graf1c and Pyk2 along with several Pyk2-PRD mutants in Hek293T cells (FIG. 8D). Graf1c is able to interact with GFP-Pyk2 and the PRD region of Pyk2 alone (GFP-PRD), but fails to bind Pyk2 if either or both of the Pyk2 PRD regions are mutated. Together, these studies show that Graf1c is an endogenous brain partner of Pyk2 with their interaction mediated through the PRD regions of Pyk2.

To define the potential functions of Pyk2-Graf1c interaction, their relative subcellular localization was assessed in neurons. XFP-tagged Pyk2 and Graf1c constructs were expressed in primary mouse hippocampal neurons (FIG. 8E). RFP-Graf1c coexpressed together with GFP (to visualize neuronal morphology) generates RFP-Graf1c specific puncta with a synaptic morphology. Coexpression of GFP-PSD95 and RFP-Graf1c confirmed that the observed RFP-Graf1c puncta are indeed synaptic (FIG. 8F). In contrast, GFP-Pyk2 displays a diffuse cellular pattern similar to that of RFP alone. Coexpression of GFP-Pyk2 with RFP-Graf1c dramatically alters Pyk2 localization and yields near complete colocalization of the two proteins. Furthermore, this colocalization depends on the PRD region of Pyk2 as the colocalization is abolished with the Pyk2 PRD region mutant GFP-PXXP2ₘᵤₜ. Without wishing to be limited by any theory, these data suggest that Pyk2 and Graf1c associate and colocalize in the dendritic spines of neurons.

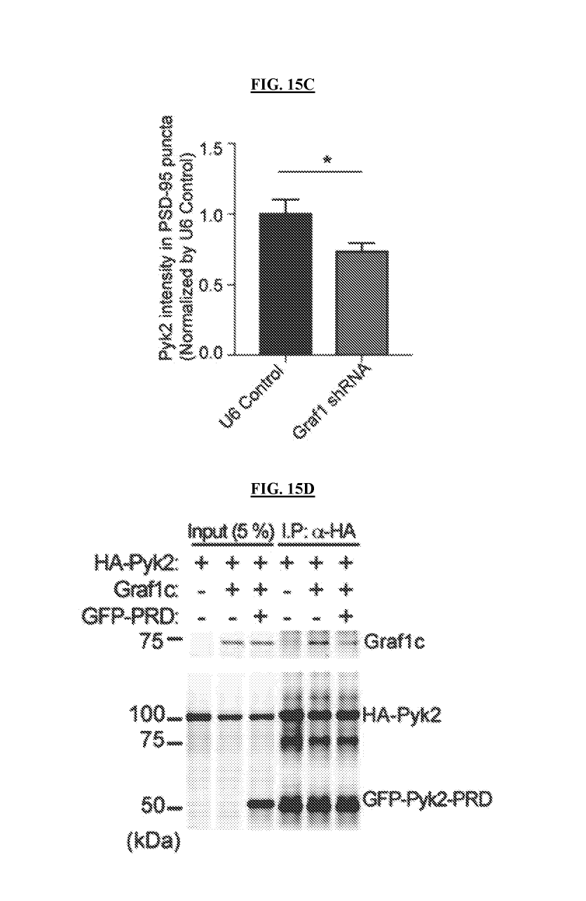

Given the striking dendritic spine colocalization of overexpressed tagged Pyk2 and Graf1c, it was determined whether endogenous Pyk2 localization to synaptic structures required Graf1c. Graf1c expression was suppressed by transfection with a plasmid expressing Graf1c-targeting shRNA and primary mouse hippocampal neurons were visualized by co-expressed myristoylated-GFP (FIGS. 15A-15B). Colocalization of endogenous Pyk2 immunoreactivity within PSD95 immunoreactive puncta significantly decreases in cells with reduced Graf1c expression (FIG. 15C). Endogenous Pyk2 interaction with Graf1c was also interrupted by coexpression of a truncated GFP-PRD consisting of the entire PRD region of Pyk2 (FIGS. 15D-15F). First, HA-Pyk2 was coexpressed together with Graf1c in Hek293T cells, and HA-Pyk2 was immunoprecipitated in the presence or absence of GFP-PRD. In a competitive manner, GFP-PRD alone is able to block the HA-Pyk2-Graf1c interaction (FIG. 15D). When overexpressed in neurons, the dominant negative GFP-PRD significantly decreases the colocalization of endogenous Pyk2 (detected by an antibody recognizing a distinct epitope) with PSD95 (FIGS. 15E-15F). Without wishing to be limited by any theory, these data show that endogenous Pyk2 and Graf1c colocalize within dendritic spines, and that their interaction is dependent on the PRD regions of Pyk2.

### Pyk2 Enrichment at Synapses is Increased by Aβo Signaling in Neurons and APP/PS1 Mice

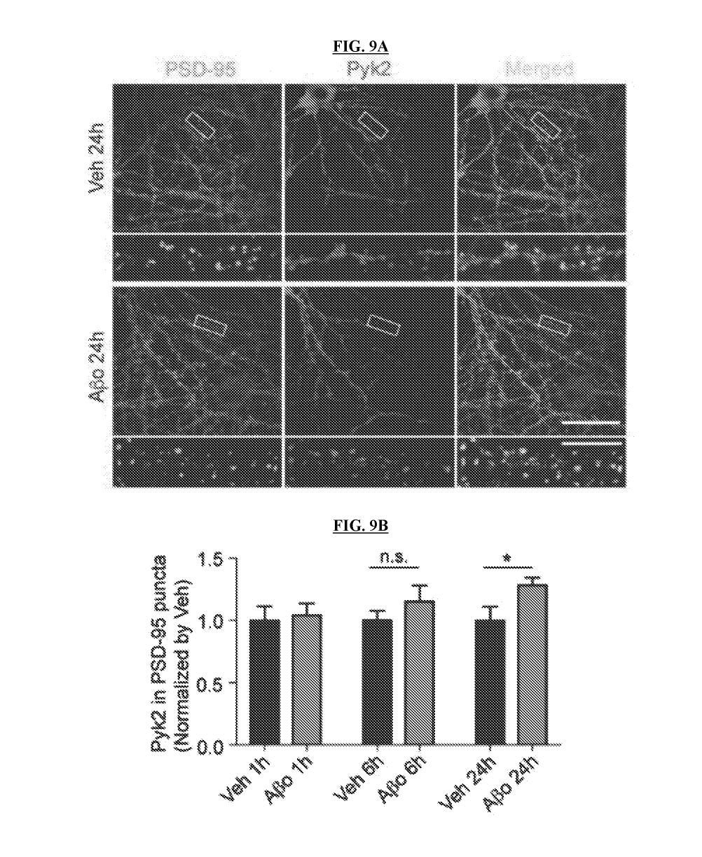

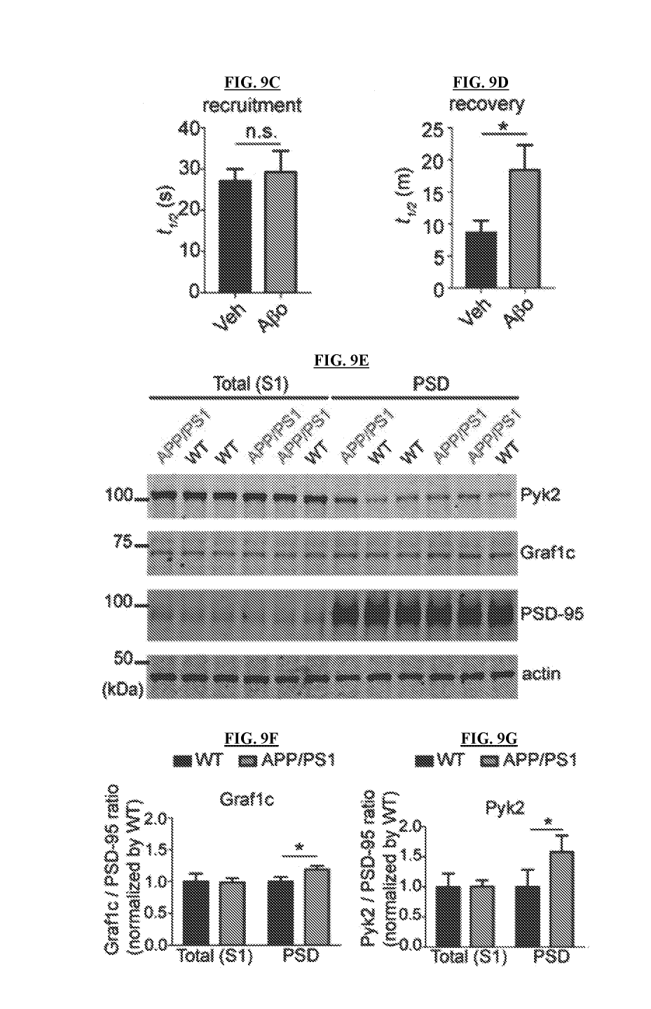

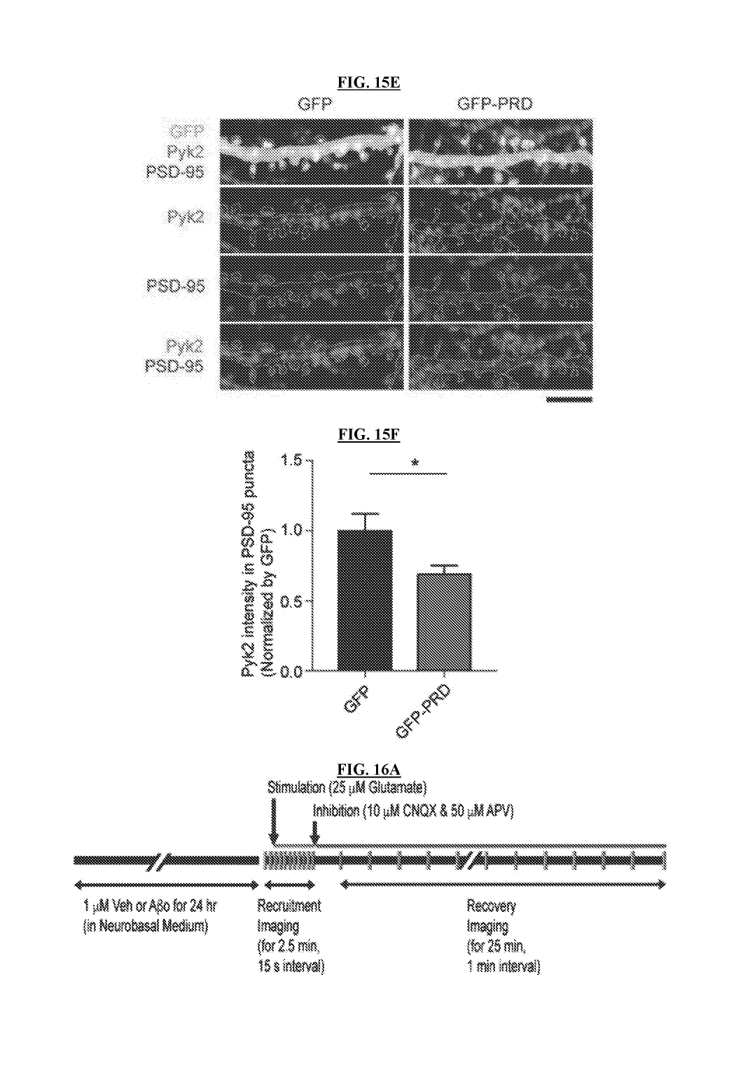

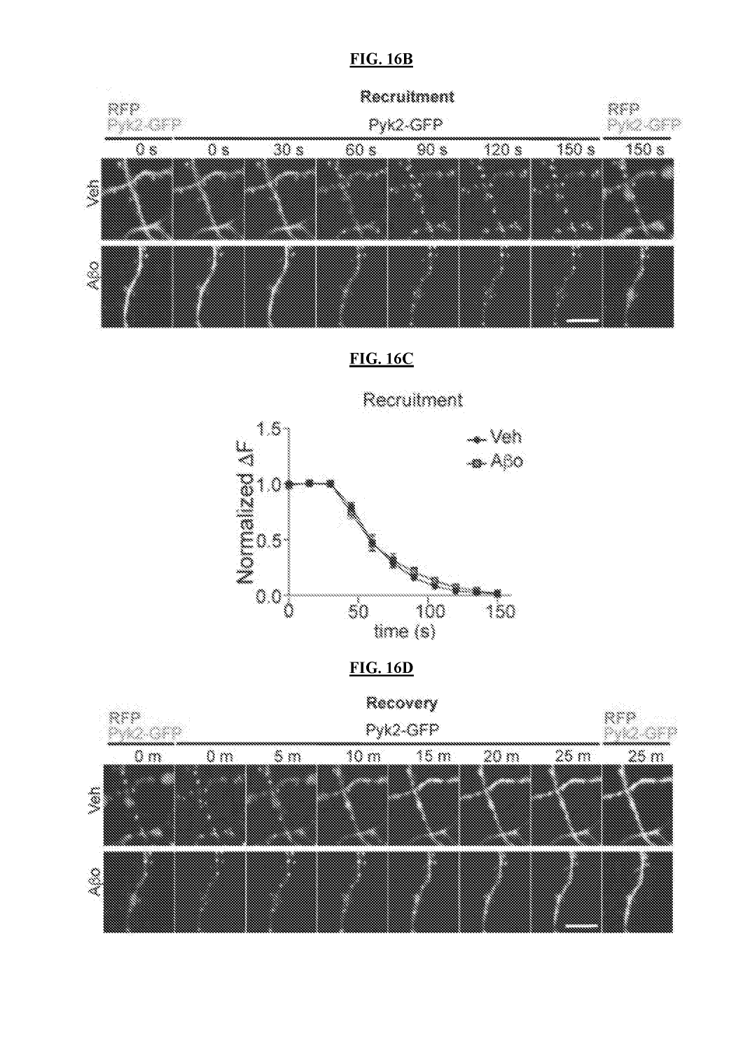

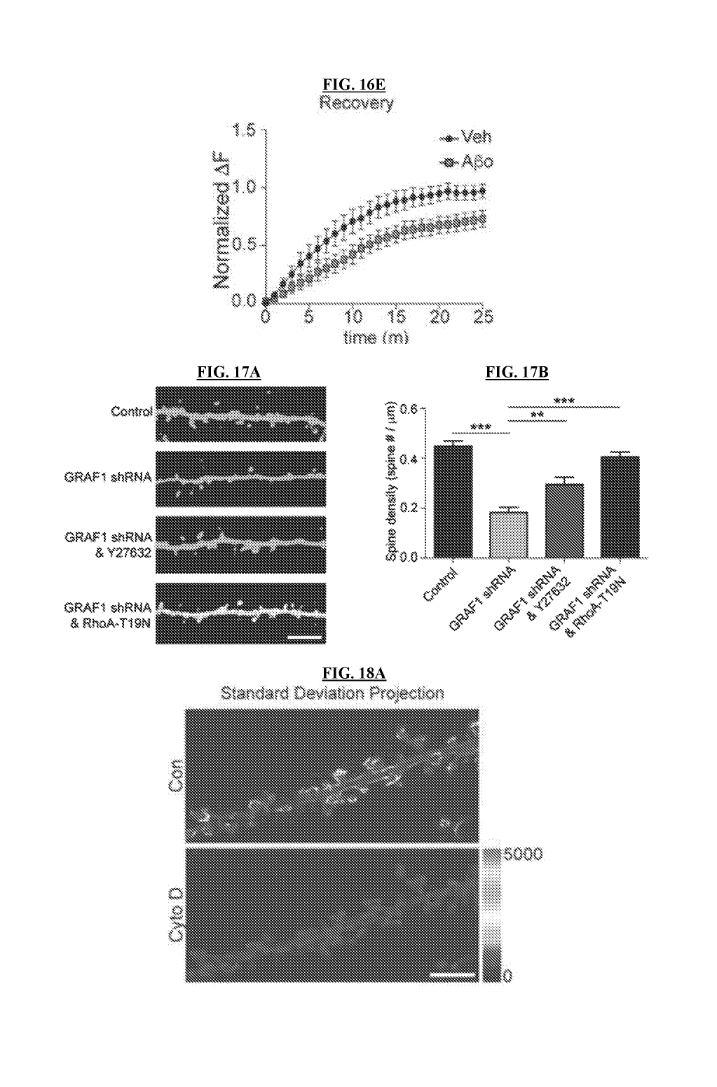

In certain non-limiting embodiments, AD pathology and Aβo signaling can regulate the localization of Pyk2 at post-synaptic sites. Primary hippocampal neurons were exposed to Aβo, and the extent of endogenous Pyk2 immunoreactivity colocalization with PSD95 puncta was determined. Treatment with Aβo, but not vehicle, for 6 hours induces a non-significant trend to increase Pyk2-PSD95 colocalization, while 24-hour exposure to Aβo significantly increases Pyk2 immunoreactivity within PSD95 immunoreactive puncta (FIGS. 9A-9B). To further explore the temporal dynamics of Aβo-dependent Pyk2 translocation to synapses vis-à-vis neuronal activity, GFP-Pyk2 translocation was monitored after glutamate stimulation (FIGS. 9C-9D & 16A-16E). Primary hippocampal neurons were treated chronically for 24 hours with vehicle or Aβo, and then time-lapse imaged recruitment of Pyk2 to synapse-like puncta using Pyk2-GFP during glutamate stimulation (FIGS. 6C & 16A-16C). The half-time (t_{1/2}) of Pyk2 recruitment to synapse-like puncta after glutamate is 25-30 seconds (FIG. 9C), and is similar for neurons treated with vehicle or Aβo (FIGS. 9C & 16B-16C). In contrast, the t₁₁₂ to recover a less punctate dendritic Pyk2 distribution after glutamate washout occurs much more slowly over 10-20 minutes and is significantly delayed for Aβo pretreated neurons compared to vehicle pretreated neurons (FIG. 9D). Furthermore, the Aβo-treated neurons never fully recover a diffuse dendritic Pyk2 distribution within 25 minutes of continuous imaging while control neurons do so (FIGS. 16D-16E). The findings reveal persistent Pyk2 recruitment to post-synaptic regions by combined Aβo and glutamate signaling. To reconfirm the synaptic enrichment of Pyk2 and Graf1c by biochemical methods, both protein levels were measured in post-synaptic density (PSD) preparations from WT and APP_{swe}/PSEN1ΔE9 (hereafter referred to as APP/PS1) mice at 9 months of age when AD related phenotypes have developed (FIGS. 9E-9G). Both Graf1c and Pyk2 levels are significantly increased in the PSD fraction of APP/PS1 mice compared to WT mice. Without wishing to be limited by any theory, these studies suggest that both Pyk2 and Graf1c are enriched at synapses in an Aβo-dependent manner.

### Pyk2-Graf1c Interaction Mediates Dendritic Spine Loss Through RhoA Activation

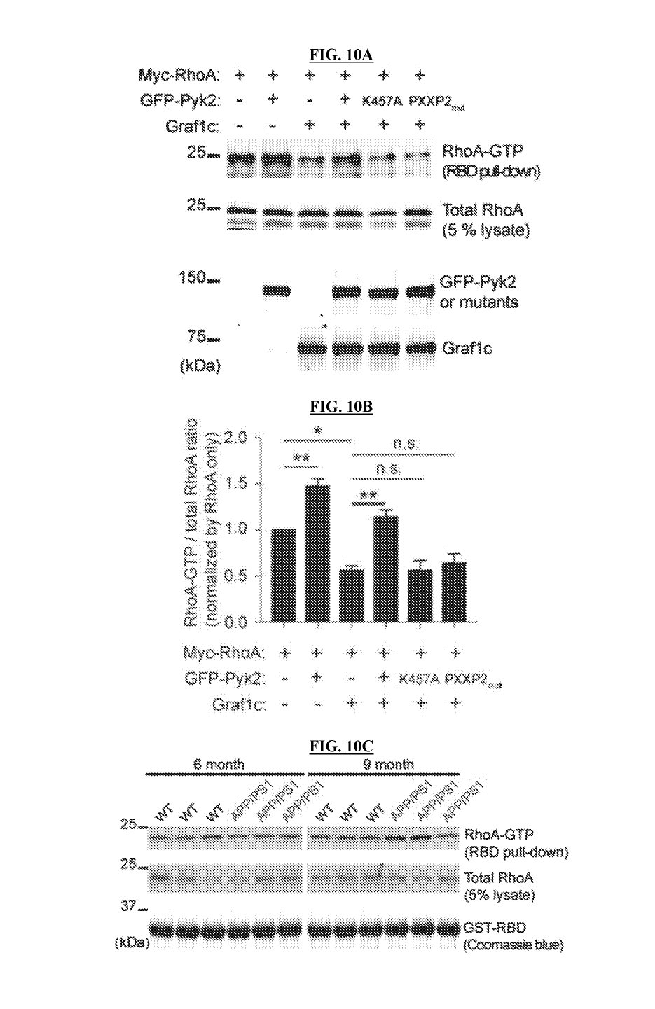

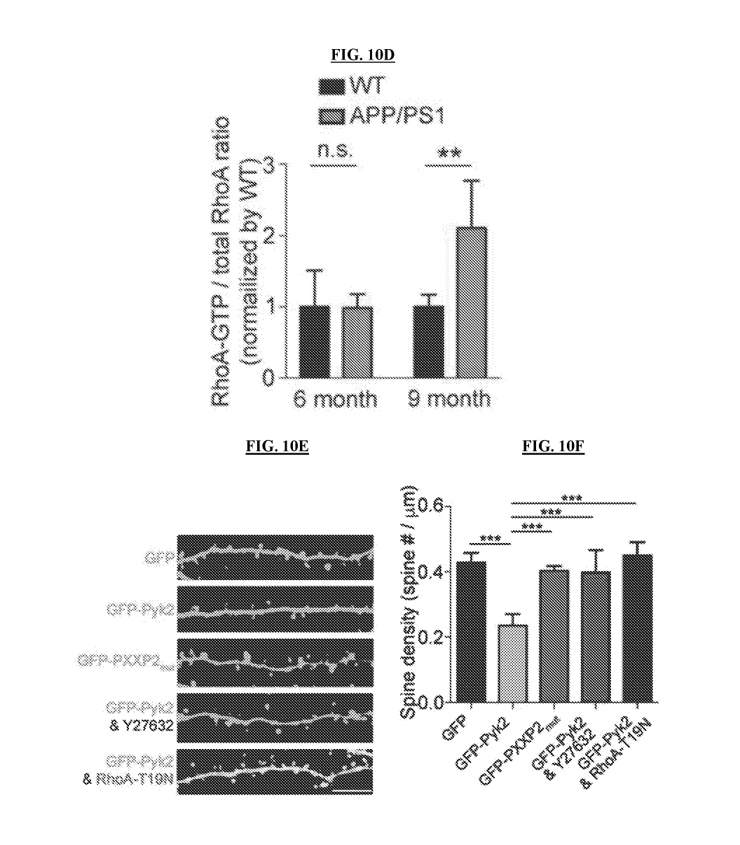

In certain non-limiting embodiments, a potential mechanism for Pyk2-mediated spine loss is the sequestration and inhibition of Graf1c by Pyk2 with a resulting increase in RhoA GTPase activity. RhoA signaling is a modulator of actin and dendritic spine dynamics. In certain embodiments, Aβo signaling can increase Pyk2-Graf1c interaction at the synapse, and this can compete with Graf1c-RhoA interaction and cause a net decrease in Graf1c-mediated inhibition of RhoA. The GST-RBD (Rhotektin RhoA-binding domain) pull-down assay was utilized to measure activated RhoA-GTP in Hek293T cells overexpressing Myc-RhoA, Graf1c, GFP-Pyk2, catalytically inactive Pyk2 (K457A), and Pyk2 PRD mutant (PXXP2ₘᵤₜ) (FIG. 10A-10B). The effect of Graf1c overexpression to reduce RhoA-GTP activity below baseline is consistent with its known GAP activity for RhoA. Coexpression of GFP-Pyk2 together with Graf1c leads to an increase in active RhoA-GTP compared to Graf1c alone, consistent with inhibition of Graf1c-GAP function. The Pyk2-dependent increase of Graf1c-suppressed RhoA-GTP does not occur with the Pyk2 mutants, K457A or PXXP2ₘᵤₜ. Thus, Pyk2 blockade of Graf1c-mediated GAP activity to increase RhoA-GTP requires both the Pyk2 kinase activity and the Graf1c-interaction. Overexpression of GFP-Pyk2 alone significantly increases RhoA-GTP levels, and this is consistent with inhibition of the low level of endogenous Graf1 present in Hek293T cells (FIG. 15D).

To investigate the in vivo and AD relevance of this pathway, the same RBD pull down assay was performed on WT and APP/PS1 forebrain lysates (FIGS. 10C-10D). At 6 months of age, this strain has low levels of Aβ accumulation and no behavioral deficits or synaptic loss, while at 9 months of age, Aβo levels rise, synapses are lost and memory function is impaired. Paralleling the temporal pattern of these phenotypes, there is a significant increase of active RhoA-GTP levels in APP/PS1 mice at nine months as compared to WT mice or to APP/PS1 mice at six months of age. RhoA activity increases as disease progresses in the APP/PS1 strain.

Next, it was evaluated whether RhoA activity is necessary for Pyk2-induced decrease in dendritic spine density. As described elsewhere herein, overexpression of GFP-Pyk2 induces loss of half of hippocampal neuron dendritic spines (FIGS. 10E-10F). Consistent with the Pyk2-Graf1c-RhoA interaction mediating spine density reduction, the Pyk2 PRD mutant PXXP2ₘᵤₜ, which fails to interact with Graf1c or regulate RhoA, does not display reduced spine density compared to GFP control. Treating neurons with the Rho/Rock pathway inhibitor Y27632, or coexpression of dominant-negative RhoA-T19N each fully rescue GFP-Pyk2-induced spine loss. Furthermore, knockdown of Graf1c expression induces significantly decreased spine density, and both Y27632 treatment and RhoA-T19N coexpression rescue Graf1c-knockdown-induced spine loss (FIGS. 17A-17B). Therefore, Pyk2 interacts with Graf1c within dendritic spines to allow increased RhoA-GTP levels and subsequent reduced synapse density.

### Pyk2 Signaling Mediates Aβo-Dependent Dendritic Spine Deficits in Primary Neurons

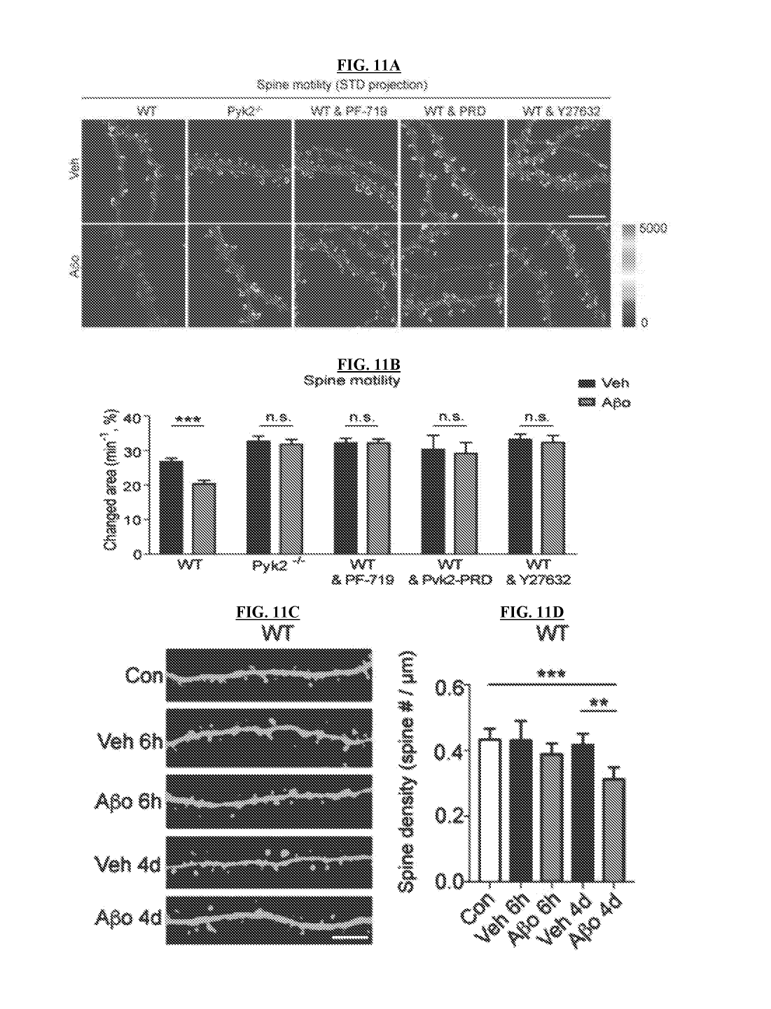

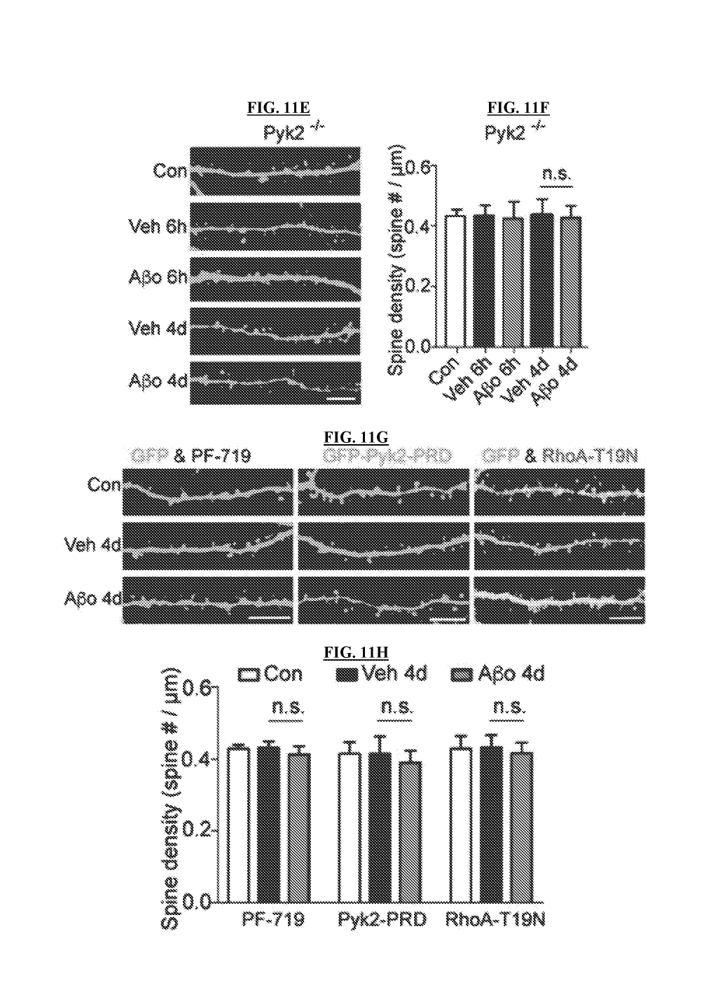

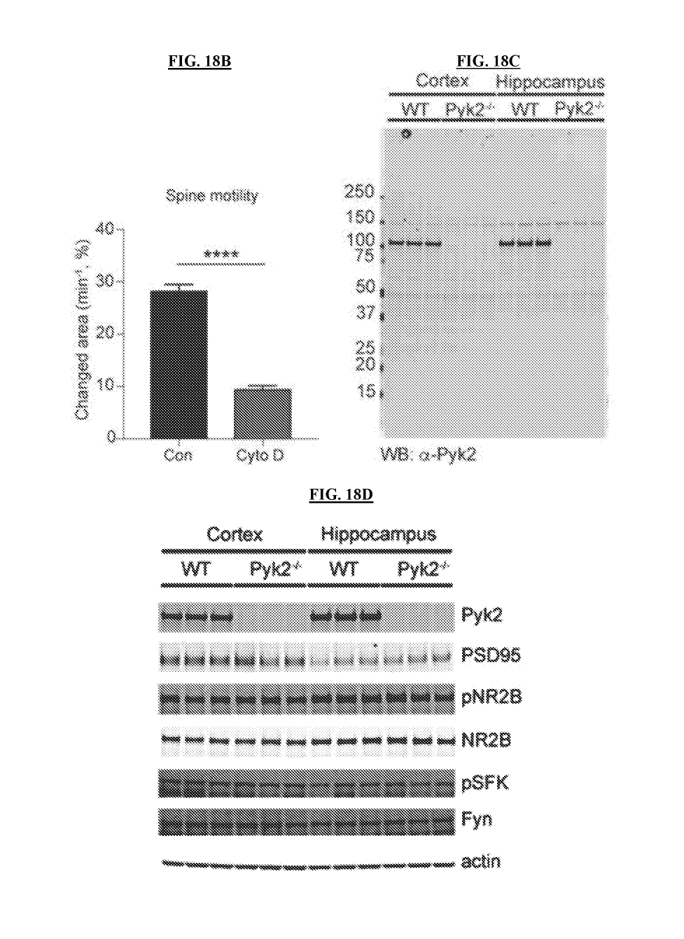

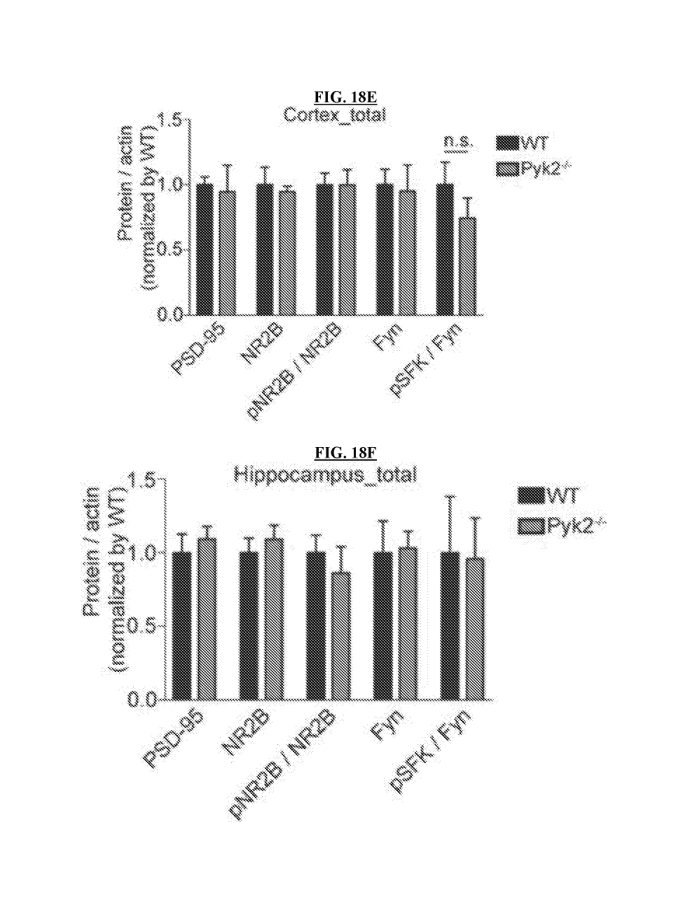

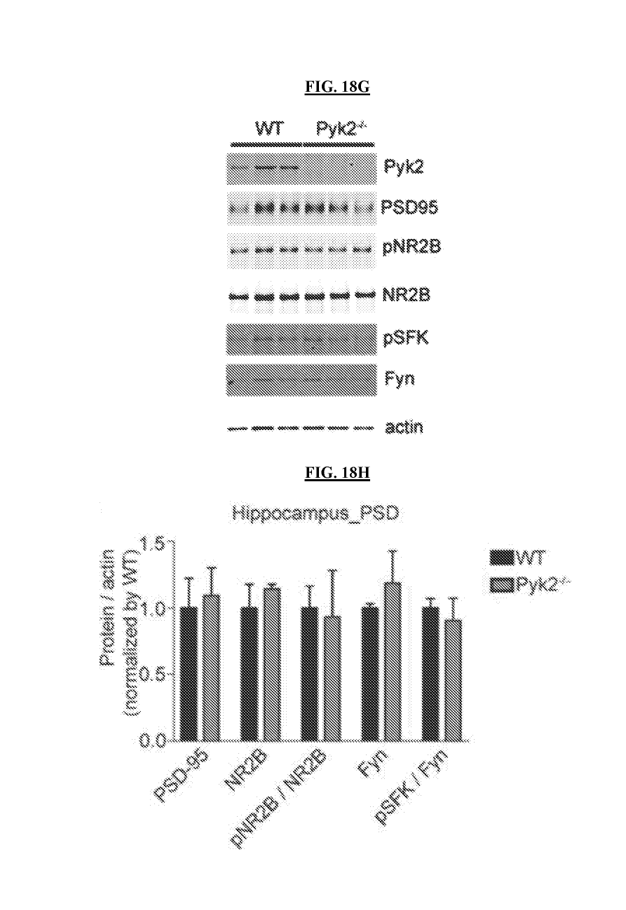

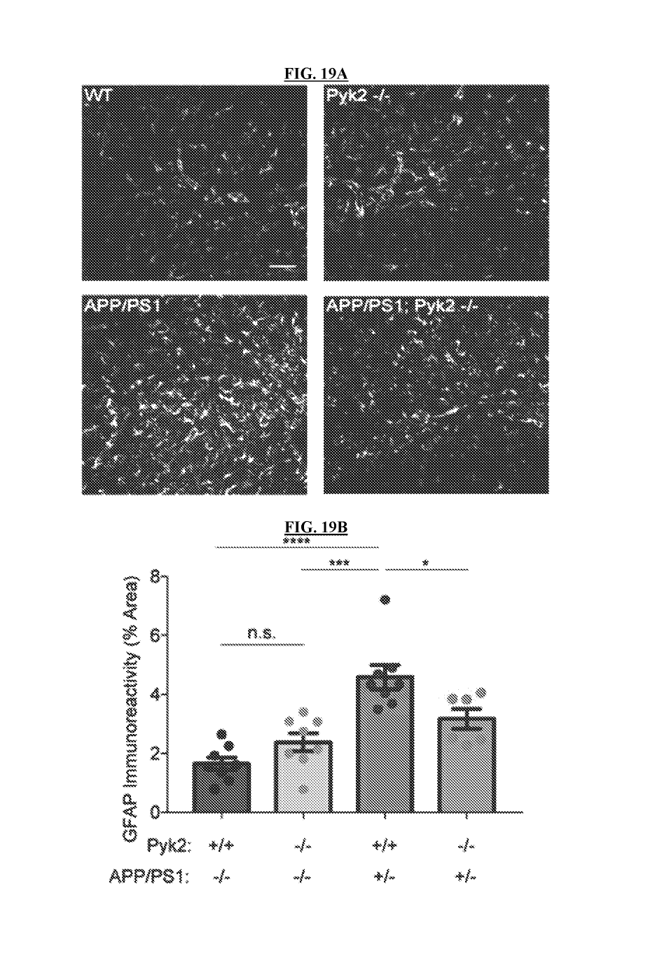

These effects of a post-synaptic Pyk2 pathway indicate that AD-mediated gain of Pyk2 function can underlie synapse loss by altering actin-based spine dynamics. Actin-dependent spine motility was monitored in primary hippocampal neurons treated with vehicle or Aβo for 24 hours (FIGS. 11A-11B). Motility was quantitated as the time-dependent standard deviation in dendritic spine profiles during 5 minutes with 0.1 Hz image captures. There is a significant decrease in spine motility from WT neurons treated with Aβo compared to WT vehicle treated neurons. To ensure that the spine motility measured is actin-dependent, neurons were treated with cytochalasin D, an inhibitor of actin polymerization, and near complete cessation of spine motility was observed (FIGS. 18A-18B). Consistent with a gain-of-function hypothesis for Pyk2, genetically deleting Pyk2, inhibiting Pyk2 with PF-719, blocking the Pyk2-Graf1c interaction with Pyk2-PRD, or inhibiting Rho/Rock signaling with Y27632 is able to rescue fully the Aβo-induced decrease in spine motility. Thus, the Pyk2/Graf1c/RhoA pathway mediates an Aβo driven deficit in spine motility.

It was considered whether the acute effects of Aβo-induced, Pyk2-mediated decrease on spine motility might be translated into alteration in spine number over longer time periods. Dendritic spine density was measured in populations of vehicle and Aβo treated primary hippocampal neuron cultures. Tracking single spines over 6 hours detects a loss of about 6-8% of spines in the presence of Aβo, but not vehicle. However, after 6 hours this loss is not significant by endpoint population analysis of dendritic spine density (FIGS. 11C-11D). Nonetheless, after 4 days, there is significant net reduction of spine density by 25% in the Aβo-treated WT cultures (FIGS. 11C-11D). In contrast, Pyk2−/− neurons treated with Aβo display no dendritic spine loss at six hours or 4 days compared to vehicle-treated controls (FIGS. 11E-11F). Consistent with the Pyk2 gain-of-function hypothesis, small molecule inhibition of Pyk2 with PF-719, blocking the Pyk2-Graf1c interaction with Pyk2-PRD, or inhibiting RhoA activity with dominant-negative RhoA-T19N is each able to rescue fully Aβo dependent spine loss (FIGS. 11G-11H). Without wishing to be limited by any theory, these data suggest that Pyk2 mediates Aβo-dependent dendritic spine deficits in primary neurons.

The Pyk2−/− cultured neurons display dendritic spine density indistinguishable from vehicle-treated WT neurons (FIGS. 11C-11F). Furthermore, multiple synaptic markers are similar in Pyk2−/− and WT neurons (FIGS. 18C-18H). Thus, Pyk2 has an essential signaling role with respect to Aβo, but is non-essential or redundant with regard to healthy brain development.

### Pyk2 Mediates Learning and Memory Deficits in APP/PS1 Mice

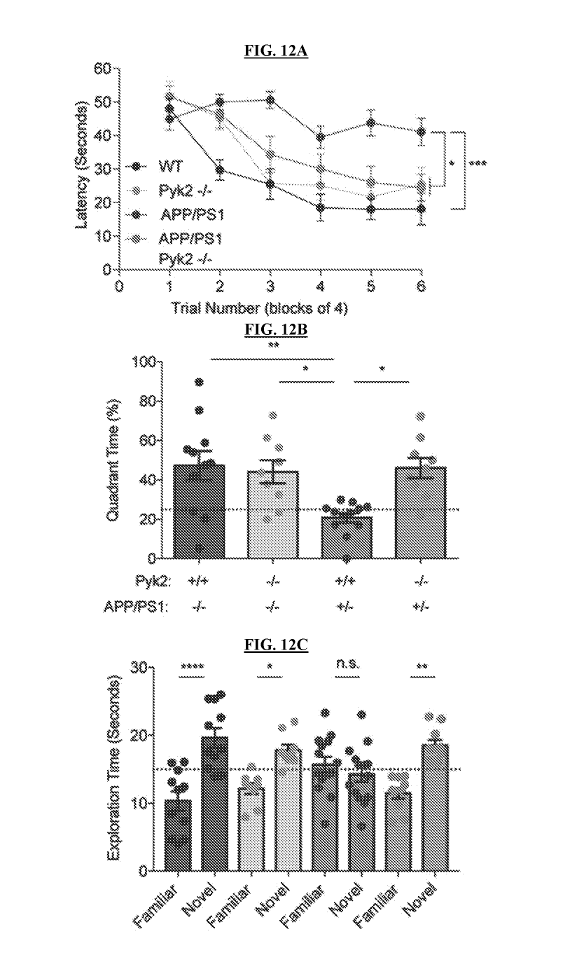

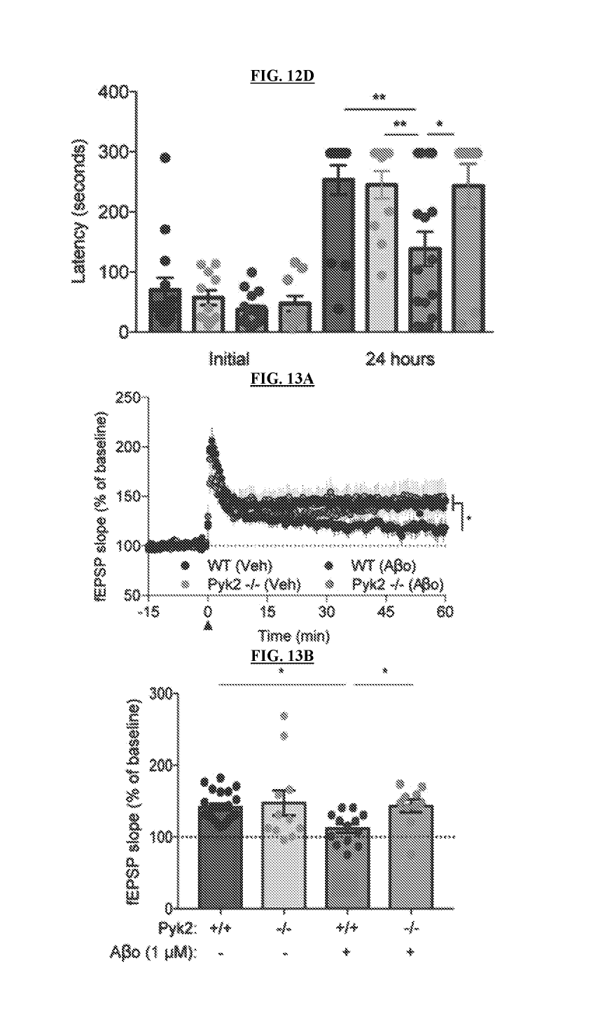

To examine in vivo evidence for Pyk2 in mediating the synaptic dysfunction of AD, APP/PS1 mice, a previously described familial AD mouse model, which displays a robust aging-dependent learning and memory deficit, were utilized. APP/PS1 and Pyk2−/− mice were crossed, producing the following genotypes: WT, Pyk2−/−, APP/PS1, APP/PS1; Pyk2−/−. APP/PS1 mice display plaque pathology by 6 months and display learning and memory deficits at 10 months of age. For this reason, cohorts of mice were examined by Morris Water Maze (MWM) test at 12 months of age when deficits are well established (FIG. 12A). The aged Pyk2−/− mice are able to find the hidden platform as quickly as WT mice during both the training and probe phases (FIG. 12A). APP/PS1 mice spend significantly more time to find the hidden platform than do WT mice. Strikingly, the APP/PS1 mice lacking Pyk2 are significantly quicker at finding the hidden platform than are APP/PS1 mice with Pyk2, and the APP/PS1; Pyk2−/− mice perform indistinguishably from WT mice. A probe trial was conducted 24 hours after the last swim to assess memory retention. APP/PS1 mice spend significantly less time than WT mice in the target quadrant, approximately the amount of time attributed to chance. In contrast, Pyk2−/− and APP/PS1; Pyk2−/− mice spend as much time in the target quadrant as do WT mice (FIG. 12B). Therefore, constitutive Pyk2 deletion does not affect healthy learning and memory, and deletion of Pyk2 rescues learning and memory deficits in this transgenic mouse model of AD.

To further characterize the role of Pyk2 in APP/PS1-mediated phenotypes, Novel Object Recognition (NOR) tests were performed on the same cohort of mice (FIG. 12C). Pyk2−/− mice prefer to explore the novel object to a similar degree as do WT mice, while APP/PS1 mice show no preference for the novel item. Deleting Pyk2 in APP/PS1 mice rescues fully the APP/PS1 deficit (FIG. 12C). As a distinct aversive learning task, the Passive Avoidance Test (PAT) was assessed for this group of mice. All groups of mice rapidly enter the dark chamber initially, wherein they receive a mild foot shock, with the same protocol repeated 5 minutes later. At 24 hours after the initial foot shock, the APP/PS1 mice show significantly less learned delay to enter the dark chamber, as compared to the WT, Pyk2−/−, and APP/PS1; Pyk2−/− mice, which all delay longer to enter the dark chamber, such that most mice never entering the dark chamber within the 5-minute observation period (FIG. 12D). Together, the data demonstrate that Pyk2 is essential for APP/PS1 mice to exhibit age-dependent learning and memory deficits.

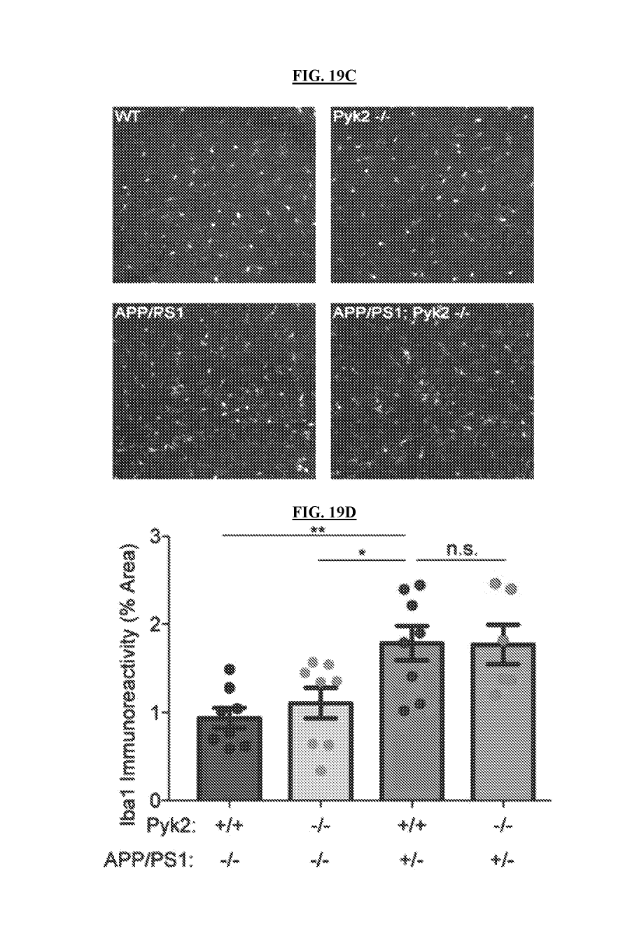

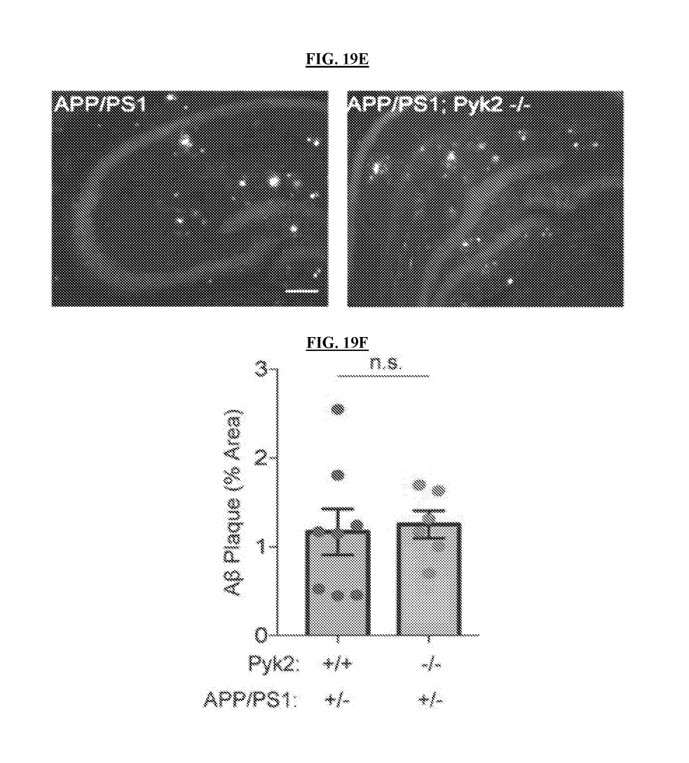

Mechanistically, Pyk2 deletion is predicted to rescue behavioral outcomes by removing a post-synaptic signaling pathway downstream of Aβ accumulation. For this reason, upstream Aβ accumulation and glial reaction are not expected to be altered, even though behavior is recovered to WT levels. Astrocytosis was examined as anti-GFAP immunoreactive area in brain sections. APP/PS1 mice display significantly greater GFAP immunoreactivity than WT mice while Pyk2 deletion does not alter the WT level (FIGS. 12A-12B). The APP/PS1; Pyk2−/− mice have an intermediate phenotype with significantly less GFAP immunoreactivity than APP/PS1 mice, but significantly more than WT mice. Microgliosis detected by anti-Iba1 immunoreactive area is increased in APP/PS1 brain sections relative to WT and Pyk2−/−. The APP/PS1; Pyk2−/− sections display as much microgliosis as do APP/PS1 mice with Pyk2, not the intermediate phenotype observed for GFAP (FIGS. 19C-19D). Similarly, plaque load remained unchanged by deletion of Pyk2 (FIGS. 19E-19F). Without wishing to be limited by any theory, microgliosis and Aβ plaque load are independent of Pyk2, while astrocytic reaction partially depends on Pyk2 in the mouse AD model.

### Pyk2 is Required for Aβo Dependent Synaptic Deficits in Long-Term Potentiation and Synapse Loss in APP/PS1 Mice

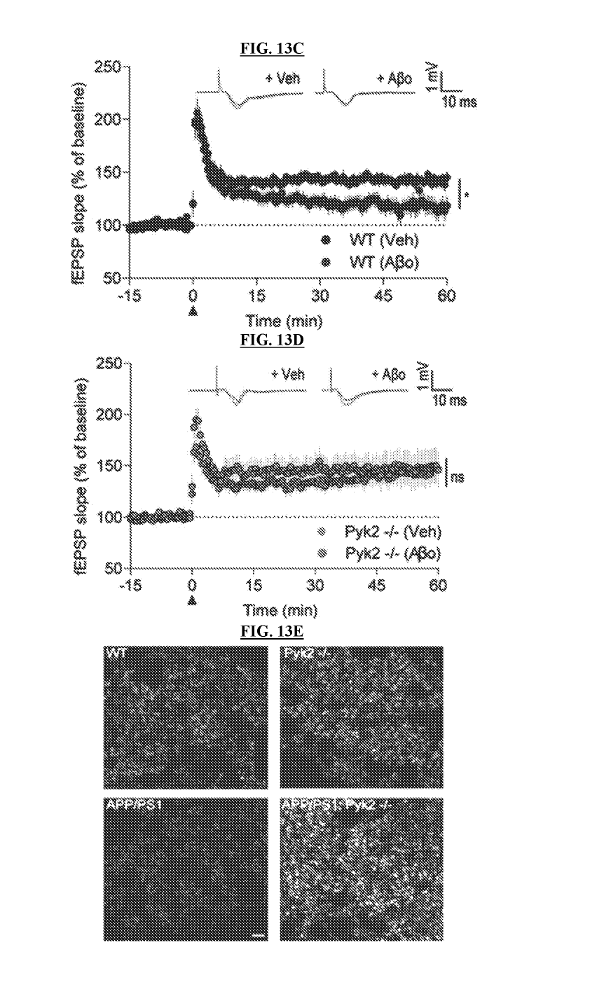

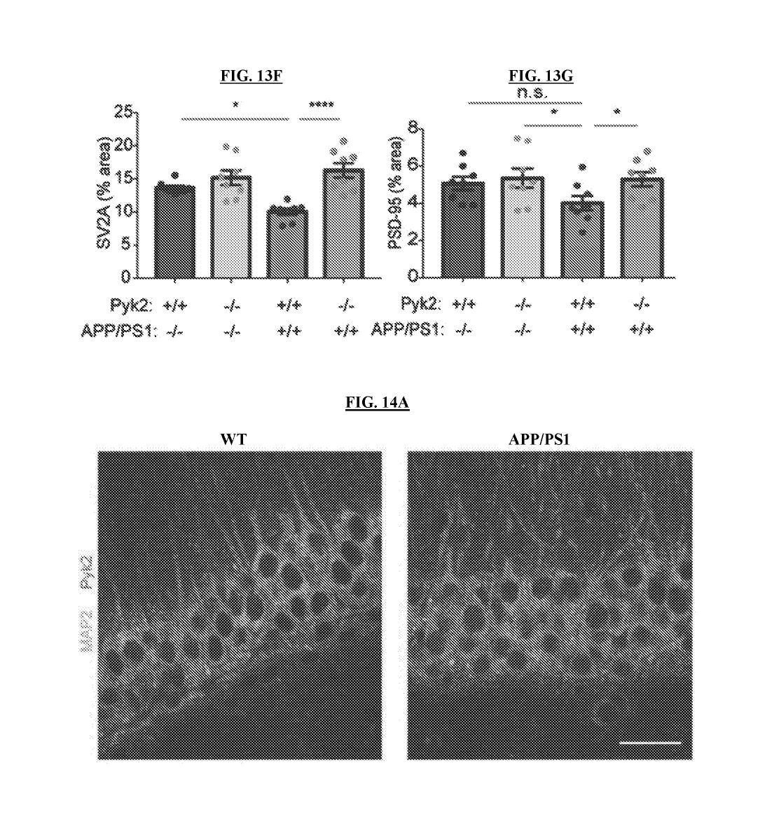

To test the hypothesis that Pyk2 mediates AD related synaptic dysfunction and loss, long-term potentiation (LTP) was measured in acute hippocampal slices exposed to Aβo. Acute treatment with Aβo suppresses LTP in acute hippocampal slices. Here, LTP was measured by recording field excitatory post-synaptic potentials (fEPSPs) at Schaffer collaterals of the CA3-CA1 hippocampal circuit from WT and Pyk2−/− hippocampal slices treated with vehicle and Aβo. As a first step, baseline input-output curves were assessed and pule facilitation in WT and Pyk2−/− were paired; no differences between genotypes were observed (FIGS. 21E-21F). Next, fEPSPs were measured before and after theta-bust stimulation (TBS), and the average of the last 10 min of recording were quantified to assess LTP. Importantly, Pyk2−/− slices treated with vehicle are not different from WT vehicle treated slices, showing that Pyk2 is not required for the induction of LTP (FIGS. 13A, 13B & 13D). Furthermore, WT slices treated with Aβo display a significant decrease in fEPSP slope during the last 10 min of recording post-TBS, compared to WT slices treated with vehicle (FIGS. 13A-13C). Strikingly, Pyk2−/− slices treated with Aβo did not display a decrease in fEPSPs compared to Pyk2−/− vehicle treated slices, and fEPSPs were similar to that of WT vehicle treated slices (FIGS. 13A, 13B & 13D). Together, these data indicate that Pyk2 is not required for the induction of LTP while, most critically, Pyk2 is essential for the Aβo-dependent deficits observed in LTP.

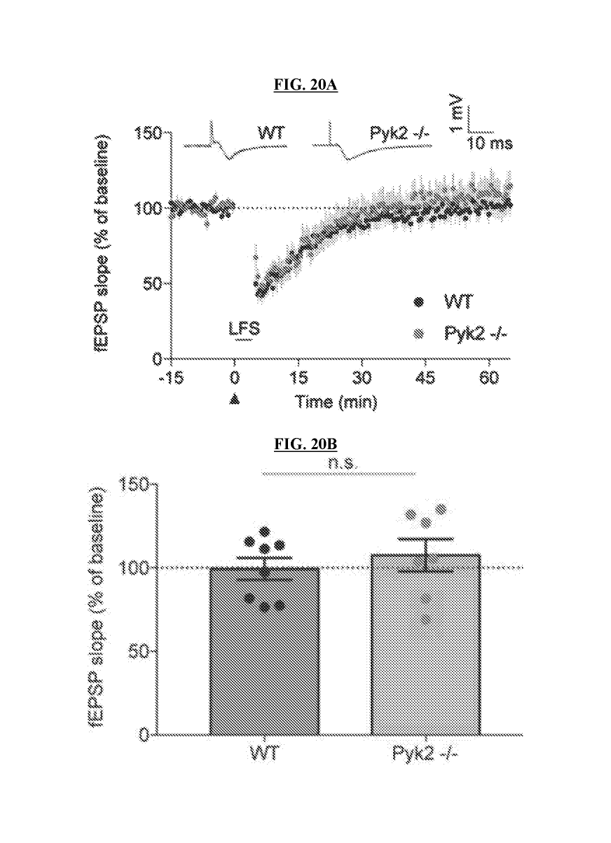

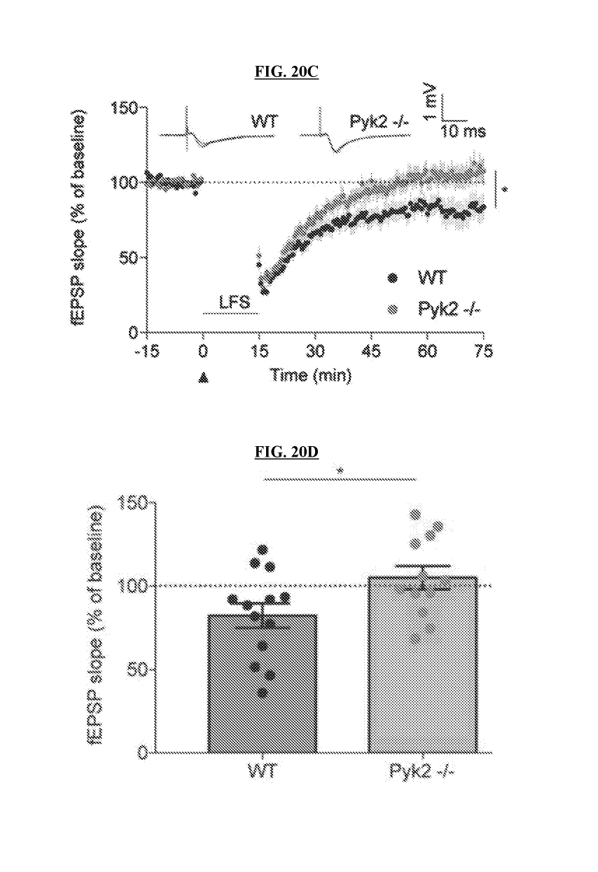

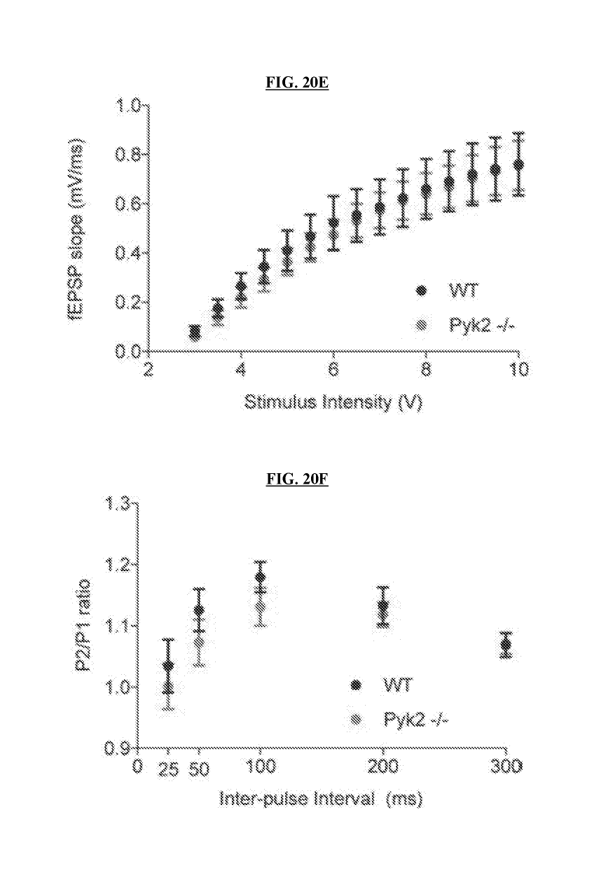

To more fully characterize the role(s) of Pyk2 in synaptic plasticity, hippocampal fEPSPs were monitored during induction of LTD with low-frequency stimulus (LFS) protocols (Li, et al., 2009, Neuron 62:788-801) (FIGS. 20A-20D). A weak LTD protocol (300 pulses at 1 Hz) does not cause LTD in either WT or Pyk2 null slices, while a more robust LTD protocol (900 pulses at 1 Hz) induces LTD within the last 20 min of recording for WT slices, but not Pyk2−/− slices (FIGS. 20C-20D). Without wishing to be limited by any theory, these findings suggest that Pyk2 has a physiological role in LTD-based synaptic plasticity even though dendritic spine density, synaptic markers, learning behavior, baseline hippocampal electrophysiology and LTP do not require Pyk2.

Since Pyk2 is necessary for the Aβo-dependent suppression of LTP in hippocampal slices and AD transgene-mediated memory deficits, it was investigated if AD-related synaptic loss in vivo requires Pyk2. Brain tissue from the 4 genotypes of aged mice described elsewhere herein was examined by immunohistology. Dentate gyms sections were stained for SV2A (presynaptic marker) and PSD-95 (post-synaptic marker) and immunoreactive area was measured in confocal images for each respective marker. The APP/PS1 mice show a significant decrease in percentage of SV2A immunoreactive area compared to WT mice (FIGS. 13E-13F) while Pyk2−/− mice do not differ from WT mice (FIGS. 13E-13F). The APP/PS1; Pyk2−/− hippocampus has the same presynaptic marker positive area as WT, and significantly more than the APP/PS1 samples. Similarly, the APP/PS1 deficit in post-synaptic PSD-95 area is fully rescued by Pyk2 deletion while Pyk2 deletion has no effect in the absence of AD-related pathology (FIG. 13G). Thus, the synaptic marker loss in aged APP/PS1 mice requires the presence of the LOAD risk gene Pyk2.

### Example 5

The present study demonstrates that activation of the AD risk gene product Pyk2 functions locally in neurons to mediate synapse loss and neural network impairment. Pyk2 protein is activated in AD tissue and localizes to neurons where its activation in concert with Fyn causes reduced dendritic spine density. The RhoA GAP protein, Graf1c, is a physical interactor of Pyk2, and RhoA activation with altered actin dynamics mediates Pyk2-induced retraction of dendritic spines. Aβ oligomeric species stimulate Pyk2 to engage this Graf1c/RhoA pathway and reduce the density of synaptic structures. Moreover, Pyk2 is essential for LTP suppression in slices by Aβo, and for memory impairment plus synapse loss in AD model transgenic mice. Thus, inhibition of Pyk2 activation is an attractive target for disease-modifying AD intervention.

Genetic analysis of rare and common variants that modify LOAD risk provides insight into the basis of bona fide human disease, as opposed to discovery techniques utilizing imperfect animal models. The challenge in AD research is linking genetic factors mechanistically to the disease process and, most importantly, to AD-related synapse loss, which is central to clinical symptoms.

As described herein, Pyk2 is unusual amongst the LOAD risk factors in possessing evidence for synaptic function in healthy brain. Indeed, the study of Pyk2 null slices show that it is essential for LTD, although synaptic density and hippocampal LTP plasticity are identical to WT. The present study sought to define Pyk2's role in synapses and AD. Pyk2 expression by neurons, subcellular localization to post-synaptic regions, and regulation by Aβo are all consistent with local synaptic action of Pyk2 as a risk gene in AD.

Pyk2 is activated in human AD and mouse models of AD, and functionally Pyk2 is required for multiple AD-related phenotypes. Without Pyk2, cultured neurons are protected from Aβo-induced dendritic spine loss, hippocampal slices are resistant to Aβo suppression of LTP, and mice with familial AD transgenes maintain synaptic markers and learning/memory behaviors despite Aβ pathology. These data implicate Pyk2 gain-of-function in AD risk.

The study of brain proteins interacting with Pyk2 identified Graf1c, and the two proteins colocalize to post-synaptic sites. Their interaction is bidirectional in the sense that Graf1c increases synaptic localization of Pyk2, while Pyk2 titrates Graf1c activity as RhoA GAP. Interaction of the two proteins requires the PRD but not the kinase domain of Pyk2. However, the kinase dependent activation of Pyk2 is essential for the ability of Pyk2 to suppress the GAP activity of Graf1c for RhoA. Graf1c recruits Pyk2 to post-synaptic sites but the basis of Graf1c localization to those sites will require further study. Fyn kinase functions synergistically with Pyk2 and also localizes in post-synaptic sites. These data place Pyk2 at the center of post-synaptic signaling complex.

Graf1c links Pyk2 function to RhoA GTPase activity. The GTP-bound active RhoA inhibits axonal and dendritic growth, to cause dendritic spine retraction and to titrate synaptic plasticity. RhoA but not cdc42 or rac1 are regulated by Graf1c. The presence of kinase active Pyk2 but not inactive Pyk2 limits the activity of Graf1c to terminate RhoA activity by stimulating the GTPase. Consistent with a central role for Pyk2 in synaptic AD related signaling, Aβo and AD transgenes also increase RhoA activation. The ability of Pyk2 overexpression and Aβo stimulation to activate RhoA are consistent with Pyk2/Graf1c complex acting via this mechanism.

Pyk2 is a tyrosine kinase capable of autophosphorylation, but Graf1c is not a substrate. While Graf1c interaction occurs via the isolated PRD domain of Pyk2, the regulation of Graf1c and RhoA-GTP requires Pyk2 kinase activity. In certain non-limiting embodiments, both Pyk2 autophosphorylation and cross-activation with Fyn contribute to Pyk2 regulation of Graf1c. Without wishing to be limited by any theory, while the presentr data show that Graf1c and RhoA play an essential role, unrelated kinase substrates of Pyk2 may also contribute to the ability of Pyk2 and Aβo to induce loss of dendritic spines.

Neither perturbation in Pyk2 nor these other signaling complex members alters the accumulation or metabolism of Aβ itself. In general, the glial reaction to Aβ pathology, including both astrocytic reaction and microgliosis, is not altered by interruption of this postsynaptic signaling Pyk2 cascade. A partial reduction in astrocytic reaction in APP/PS1 mice lacking Pyk2 was observed. Without wishing to be limited by any theory, this can be explained directly by reports of astrocytic Pyk2 function or it can be indirect with protection of synapses reducing astrocytic reaction.

The present studies indicate that Pyk2 inhibition is a target for disease-modifying AD treatment. In one aspect, human genetic risk studies validate its relevance. In another aspect, the present biochemical analysis shows that Pyk2 activity couples to a post-synaptic signaling pathway for synapse loss that is central to brain dysfunction in AD.

### Methods

### Animals

The mouse strains used (APP_{swe}/PSEN1ΔE9 mice on a C57BL/6J background, Gimbel, et al., 2010, J. Neurosci. 30:6367-6374) were purchased from Jackson Laboratory (Bar Harbor, Me.). The Pyk2−/− mice were generated (Okigaki, et al., 2003, Proc. Natl. Acad. Sci. USA 100:10740-10745), and provided on the C57Bl6J background after 10 backcrosses. All experiments utilized littermate control mice with no preference for male or female mice. The percentage of female mice in the APP/PS1 and the APP/PS1, Pyk2−/− groups was 42% and 56%, respectively, for FIGS. 12A-12D & 13A-13G).

### Plasmid DNA Constructs

Full-length wild type (WT) Pyk2, K457A, PXXP1mut, PXXP2mut, ΔPRD, and PRD mutants were subcloned into AAV-CAG-GFP vector (gift from K. Svoboda, Janelia Research Campus, Addgene plasmid #28014) for GFP tagging on N-terminus or AAV-CAG-tagRFP vector, modified from AAV-CAG-GFP by replacing the GFP with tagRFP for tagRFP tagging on N-terminus. cDNA encoding human Fyn was subcloned into pcDNA3 with an N-terminal extension encoding a flag tag. Human Graf1a and Graf1c isoforms were generated from Graf1b isoform (DNASU plasmid repository, clone ID HsCD00639889) by PCR, subcloned into AAV-CAG-tagRFP or pcDNA3. pRK5-Myc-RhoA-wt and pRK5-Myc-RhoA-T19N were gift from Gary Bokoch (Addgene plasmid 12962 and 12963). GFP expression plasmid was generated from AAV-CAG-GFP vector by the insertion of stop codon after GFP open reading frame (ORF). The myristoyl-GFP plasmid has been described (Um, et al., 2012, Nat. Neurosci. 15:1227-1235).

### Culture and Transfection of Mouse Hippocampus Neurons

Cultured hippocampal neurons were prepared from embryonic day 17 fetal C57/BL6J mice. Briefly, dissected hippocampi were dissociated with papain and plated on poly-D-lysine coated 18 mm glass coverslips or culture plates with plating medium (Neurobasal-A medium supplemented with 2% B-27, 2% FBS, 1% GlutaMax, and 1 mM sodium pyruvate, all from Thermo Fisher Scientific). Four hours after plating, all medium was replaced with FBS free culture medium (Neurobasal-A medium supplemented with 2% B-27, 1% GlutaMax, and 1 mM sodium pyruvate) and then 50% replacement every 7 days. Neurons were transfected before plating by Amaxa Nucleofector (shRNA efficiency test) or at DIV 17 by modified calcium-phosphate method. Briefly, 7 μg of plasmid DNA and 9 μl of 2 M CaCl₂ were mixed in distilled water to a total volume of 75 and the same volume of 2×BBS (50 mM BES, 280 mM NaCl, and 1.5 mM Na₂HPO₄, pH 7.1) was added. Original cultured medium was completely replaced by transfection medium (MEM, 1% GlutaMax, 1 mM sodium pyruvate, 0.6% glucose, and 10 mM N-2-hydroxylpiperazine-N′-2-ethane sulfonic acid (HEPES), pH 7.65), and plasmid DNA mixture was added to the neurons in transfection medium. After incubation for 90 min in 5% CO₂ incubator, washed twice for 20 min with transfection medium (pH 7.35) and then returned to the original culture medium.

### Human Brain Tissue Collection

Human post-mortem brain tissue was collected and stored at 80° C. until experimental use. The neuropathological features of Alzheimer's disease in patient's brains were confirmed by classifying them as Braak stage V or higher with a ‘frequent’ CERAD (Consortium to Establish a Registry for Alzheimer's Disease) neuritic plaque score. Healthy controls were classified as Braak stage 0-II with a CERAD neuritic plaque score of ‘none’ or ‘sparse’.

Human frontal cortex was homogenized in three times the tissue weight in TBS containing PhosSTOP (Roche) and cOmplete Mini protease inhibitor cocktail (Roche). Homogenates were centrifuged for 30 minutes at 100,000×g and 4° C. The supernatant was again centrifuged for 30 minutes at 100,000×g and 4° C. The supernatant was collected as TBS-soluble fraction. Proteins were extracted from the remaining pellets by centrifugation in three times the original brain tissue weight in TBS+1% Triton X-100 for 30 minutes at 100,000×g and 4° C. The supernatant was collected and again centrifuged for 30 min at 100,000×g and 4° C. The protein concentration in the supernatant was measured by Bradford assay (Bio-Rad Protein Assay).

### Hek293T Cell Culture and Transfection

Human embryonic kidney 293T (Hek293T) cells were cultured in DMEM containing 10% FBS at 5% CO₂ and 37° C. incubator and transfected using Lipofectamine 3000 reagent (Thermo Fisher Scientific).

### Immunoprecipitation

Lysates from transfected Hek293T cells or mouse forebrains were extracted with modified radio immune precipitation assay (RIPA) buffer (1% Triton X-100, 50 mM Tris, 150 mM NaCl, 1 mM EDTA, protease inhibitor cocktail (Roche), and phosphatase inhibitor cocktail (Roche)) and quantitated the protein concentration by BCA assay kit (Thermo Fisher Scientific). 1 mg of lysates and 1 μg of appropriate antibody (anti-GFP antibody, Abcam ab290; anti-flag, Sigma-Aldrich; anti-Pyk2, Abcam ab32571) mixtures were incubated for 2 hours at 4° C., 20 μl Protein A/G agarose beads (Thermo Fisher Scientific) was added, and incubation continued for 1 hour on rotator. The immunoprecipitated complexes were washed 3 times with RIPA buffer, eluted in sodium dodecyl sulfate-polyacrylamide gel electrophoresis (SDS-PAGE) sample loading buffer from beads, and then resolved by SDS-PAGE and immunoblotted.

### Immunoblotting

Proteins were resolved using precast 4-20% tris-glycine gels (Bio-Rad) and transferred to nitrocellulose membrane by iBlot Gel Transfer Device (Thermo Fisher Scientific). The membranes were blocked in blocking buffer (Rockland MB-070-010) for fluorescent immunoblot for 1 hour at room temperature (RT) and incubated overnight in primary antibodies at 4° C. The following antibodies were used: rabbit anti-Pyk2 (Abcam ab32571, 1:1000), mouse anti-Pyk2 (Santa Cruz Biotechnology, SC130077, 1:1000), anti-phospho-Pyk2 (Y402) (Abeam ab131543, 1:1000), anti-GFP (Abcam ab13970, 1:5000), anti-Fyn (Santa Cruz Biotechnology sc71133, 1:1000), anti-phospho-SFK (Cell Signaling Technology A2066, 1:1000), anti-flag (Sigma-Aldrich F1804, 1:1000), anti-Graf1 (Abcam ab137085, 1:1000), anti-PSD-95 (Synaptic Systems 124-002, 1:2000), anti-Actin (Sigma-Aldrich A2066, 1:2000), anti-Myc (Cell Signaling Technology 2276, 1:5000), anti-RhoA (Cytoskeleton ARH04, 1:1000). After primary antibody incubation, the membranes were washed and applied appropriate secondary antibodies (Odyssey donkey anti-rabbit, anti-mouse, or anti-chicken IRDye 680 or 800 conjugates, LI-COR Biosciences) for 1 hour at RT. The proteins were visualized using a LI-COR Odyssey infrared imaging system and quantified with Image Studio Lite software.

### Immunocytochemistry

Cultured neurons were fixed in 4% paraformaldehyde/4% sucrose/phosphate-buffered saline (PBS) for 15 min, permeabilized for 5 min in 0.25% Triton X-100/Tyrode's solution (136 mM NaCl, 2.5 mM KCl, 2 mM CaCl₂, 1.3 mM MgCl₂, 10 mM Na-HEPES, 10 mM D-glucose, pH 7.3) and then incubated in 10% BSA for 30 minutes at 37° C. for blocking. Primary antibodies (anti-PSD-95, Millipore MAB1596, 1:1000; anti-Pyk2, Abcam ab32571, 1:1000; anti-Myc Cell Signaling Technology 2276, 1:2000) diluted in 3% BSA/Tyrode's solution were incubated for 2 hours at 37° C. Then, appropriate secondary antibodies (Alexa-fluor-488 or Alexa-fluor-568 conjugated donkey anti-mouse IgG or anti-rabbit-IgG, Thermo Fisher Scientific, 1:1000) were diluted in 3% BSA/Tyrode's solution and applied for 45 min at 37° C.

### Pyk2 Binding Protein Identification

Anti-GFP antibody captured immunoprecipates from 1 mg protein from GFP, GFP-Pyk2, or GFP-K457A expressed Hek293T cell lysates with or without 5 mg mouse brain lysate mixtures were separated by SDS-PAGE and silver-stained using silver-staining kit (Pierce 24600). Pyk2-specific binding protein in mouse brain or kinase dead K457A mutant specific binding protein bands were excised from the stained gel and tryptic peptides were identified by LC-MS/MS analysis.

### Aβ₁₋₄₂ Preparation

Aβ₁₋₄₂ peptide was synthesized. Aβ₁₋₄₂ oligomers (Aβo) were prepared in specially formulated glutamate-free F-12 to avoid direct stimulation of cultured neurons (Lauren, et al., 2009, Nature 457:1128-1132). Concentration of Aβo are expressed in monomer equivalents, with 1 μM total Aβ₁₋₄₂ peptide corresponding to approximately 10 nM oligomeric species. Each new preparation of Aβo was confirmed to be >95% HMW soluble oligomers by size exclusion chromatography.

### Mouse Brain Tissue Collection for PSD Fractionation and Biochemistry

After rapid decapitation, whole forebrain or cortex and hippocampus were quickly dissected and homogenized in ice-cold SYN-PER™ reagent (Thermo Fisher Scientific) containing complete protease inhibitor cocktail (Roche) and phosphatase inhibitor cocktail (Roche) with Teflon homogenizer. Homogenized brain extract was spun at 1,000×g for 10 minutes to remove pelleted nuclear fraction (P1). Supernatant (S1) was centrifuged at 15,000×g for 15 minutes to collect the crude membrane pellet (P2). P2 was resuspended in HEPES-buffered sucrose (0.32 M sucrose, 4 mM HEPES, pH 7.4, complete protease inhibitor cocktail (Roche), phosphatase inhibitor cocktail (Roche)) and then spun at 15,000×g for 15 minutes to yield the washed crude synaptosomal fraction (P2′). Lysed resulting pellet by hypo-osmotic shock in cold H₂O with protease and phosphatase cocktail inhibitor and homogenized again with Teflon homogenizer then rapidly adjust to 4 mM HEPES. After hypo-osmotic lysis, samples were spun at 25,000×g for 20 minutes to separate the supernatant (S3) and pellet (P3). The P3 suspension in HEPES-buffered sucrose was loaded onto a discontinuous sucrose gradient (0.8 M-1 M-1.2 M sucrose solution in 4 mM HEPES, pH 7.4), followed by centrifugation for 2 hours at 150,000×g. The synaptosome fraction between 1 M and 1.2 M sucrose layer was collected and adjusted to 4 ml with 4 mM HEPES pH7.4, spun at 150,000×g for 30 minutes to yield the pellet (SPM). SPM was resuspended in lysis buffer (50 mM HEPES, 2 mM EDTA, 1% Triton X-100, protease inhibitor cocktail, and phosphatase inhibitor cocktail) and incubated for 15 minutes. The suspension was spun at 32,000×g for 20 minutes. The resulting pellet was extracted again with 0.5% Triton X-100 lysis buffer for 15 minutes, and spun again at 200,000×g for 1 hour. Resulting pellet (PSD) was analyzed by immunoblot.

### RBD Pull-Down Assay

GST tagged Rhotekin RBD (GST-RBD) expression plasmid was transformed into BL-21 (DE3) Escherichia coli (Agilent) and the cells were cultured in 2×YT medium with ampicillin until OD₆₀₀ reached 0.6 at 37° C., and then protein expression was induced with 0.5 mM IPTG for 6 hours at 25° C. Protein expression induced cells were lysed in lysis buffer (1% Triton X-100, 150 mM NaCl, 20 mM Tris pH7.4, 1 mM MgCl₂, 1 mM EGTA, 1 mM PMSF) with sonication and centrifuged at 20,000×g for 15 min at 4° C. Supernatant was incubated with Glutathione Sepharose 4B beads (GE Healthcare) for 1 hour. GST-RBD immobilized beads were applied to lysates from transfected Hek293T cells or mouse brain lysates (lysis buffer: 1% Triton X-100, 150 mM NaCl, 50 mM Tris pH7.4, 10 mM MgCl₂, protease inhibitor cocktail (Roche), and phosphatase inhibitor cocktail (Roche), incubated for 1 hour at 4° C. and then analyzed by SDS-PAGE and immunoblotting.

### Image Acquisition and Analysis of Dendritic Spine Density

GFP, GFP-Pyk2, and GFP-K457A transfected neurons were fixed at DIV 21. In some experiments, GFP-Pyk2 expressed neurons were incubated immediately in 1 μM PF-719 after transfection. For Aβo induced spine loss test, Aβo (1 μM monomer, 10 nM oligomer estimate) or Vehicle (Veh) were applied to GFP, GFP-Pyk2-PRD, and GFP with Myc-RhoA-T19N expressed neurons at DIV 17 and replaced 50% culture medium with fresh Aβo or Veh included conditioned culture medium every 24 hours for 4 days. For 6 hour incubation, Aβo or Veh were applied to DIV 21 neurons without media change. Neurons were fixed and imaged with a 40× objective oil lens on a Nikon Eclipse Ti Spinning Disk Confocal Microscope driven by Volocity software (PerkinElmer). Images were obtained as a 1 μm Z-stack with 0.5 μm spacing using a 488 laser. All imaging and analyses were completed by an observer unaware of genotype or treatment group.

Analysis and quantification of data were performed with Volocity software after max intensity projection. The numbers of dendritic spines were counted manually to estimate the density of primary or secondary dendritic branch by observer unaware of drug or genotype. For each condition, at least 3 dendrites were measured from each neuron and 5-7 neurons were imaged per coverslip. Myc-RhoA-T19N expression was confirmed by immunostaining with anti-Myc antibody and imaging with a 568 laser. All imaging and analyses were completed by an observer unaware of genotype or treatment group.

### Image Acquisition and Analysis of Pyk2 Localization in Immunostained Neurons

After Aβo (1 μM monomer, 10 nM oligomer estimate) or Veh treatment for 1 hour, 6 hours, and 4 days, DIV21 low density cultured neurons were immunostained with appropriate antibodies and imaged with a 60× oil immersion lens on a same microscope as for dendritic spine imaging. Postsynaptic area was selected from PSD-95 images by a pre-defined computer script using a constant threshold value, and then the average fluorescence intensity was measured for Pyk2 within each PSD-95-positive area using Volocity software. All imaging and analyses were completed by an observer unaware of genotype or treatment group.

### Image Acquisition and Analysis of Live Neurons

For live-cell imaging of GFP-Pyk2 recruitment and redistribution in response to glutamate stimulation, DIV 18 hippocampal neurons were transfected with GFP-Pyk2 and tagRFP. Aβo (1 μM monomer, 10 nM oligomer estimate) or Veh was applied to DIV 19 transfected neurons for 24 hours and cells were mounted in a magnetic perfusion chamber (Live Cell Instrument, AC-B18) on the stage of a Nikon Eclipse Ti Spinning Disk Confocal Microscope and perfused with Tyrode's solution using peristaltic pump with 1 ml/min rate. Time-lapse images were acquired every 15 s for 2.5 min with 25 μM glutamate treatment after the 3^{rd} time point for recruitment imaging. After the indicated delays, the cultures were perfused with 10 μM 6-cyano-7-nitroquinoxaline-2,3-dione (CNQX) and 50 μM DL-2-amino-5-phosphonovaleric acid (APV) in glutamate-free Tyrode's solution and images acquired for recovery imaging over 25 min with 1 min intervals. Quantitative measurements of the fluorescence intensity at individual regions of interest (ROIs) were obtained by averaging pixel intensities of a selected area using Volocity software. Background ROIs outside the dendritic area were subtracted. Fluorescence intensity change traces were obtained by subtracting the intensity of last frame from recruitment time-lapse image (recruitment) or first frame from recovery time-lapse image (recovery) then normalized to the average intensity of the first three frames from recruitment time-lapse image and averaged. The decay (recruitment) and expansion (recovery) of GFP-Pyk2 intensity curves were fitted by single exponential function and calculated the half time (t_{1/2}) using Prism 7 software. All and analyses were completed by an observer unaware of genotype or treatment group

To measure the dendritic spine motility, myristoylated-GFP expressing DIV 21 neurons from WT or Pyk2−/− mice were incubated with Aβo (1 μM monomer, 10 nM oligomer estimate) or Veh for 24 hours and mounted in a magnetic chamber (Live Cell Instrument, CM-B18-1) on the stage of a Nikon Eclipse Ti Spinning Disk Confocal Microscope. Time-lapse images were acquired for 5 minutes with 10 second intervals in Tyrode's solution using 60× objective lens driven Volocity software. Time-lapse images (31 time frames over 5 min) were subjected to standard deviation (STD) projection to visualize spine motility in single image and colorized with thermal scale using ImageJ software (NIH). Quantitative measurements of spine motility by calculating the changed area at individual dendritic spines were conducted by using ImageJ software by an observer unaware of treatment group. Spine motility was monitored as changed area by subtracting thresholded, binarized individual images from the merged area for every 30 second time frame over 5 minutes. In some experiments, 1 μM PF-719 or 10 μM Y-27632 were applied with or without Aβo for 24 hours.

### Brain Tissue Collection

Immunohistology

Mice were euthanized and brains were dissected, divided at the midline into two hemispheres where one hemisphere was drop-fixed in 4% paraformaldehyde (PFA) for 24 hours. The other hemisphere was flash frozen and stored at −80° C. Following PFA fixation, brains were stored in Phosphate Buffer Saline (PBS) with 0.05% Sodium Azide. For immunostaining, brains were cut into 40 μm coronal sections using a Leica (Wetzlar, Germany) WT1000S Vibratome. Sections were stored in PBS with 0.05% Sodium Azide at 4° C. until staining.

### Immunohistology

Free-floating 40 μm sections were washed in a 24-well plate once with 0.1% PBS-Triton X-100 for five minutes (unless otherwise noted for a particular antibody). Subsequently, sections were blocked with 10% Normal Horse Serum (NHS) in PBS for one hour at room temperature. Sections were then incubated in primary antibody with 4% NHS in PBS overnight at 4° C. The following primary antibodies were used: rabbit anti-β-Amyloid (Cell Signaling Technologies, #2454, 1:250), chicken anti-GFAP (Abcam ab4674, 1:500), rabbit anti-Iba1 (Wako Chemicals #019-19741, 1:250), rabbit anti-PSD-95 (Invitrogen, 51-6900, 1:250; this staining required antigen retrieval with for 5 min with 1% Sodium Dodecyl Sulfate (SDS) in PBS at 90° C.), rabbit anti-SV2A (Abcam ab32942, 1:250). Sections were washed three times after primary incubation with PBS for 10 minutes each. Sections were then incubated with fluorescent secondary antibodies (donkey anti-rabbit, donkey anti-rat, or donkey anti-chicken; Invitrogen, Alexa Fluor, 1:500) for one hour at room temperature. Samples were washed three times in PBS for 10 minutes each. To quench lipofuscin autofluoresence for Iba1 staining, samples were incubated in 10 mM CuSO₄ in ammonium acetate for 15 minutes after secondary antibody. The samples were washed once with PBS and mounted onto glass slides (Superfrost Plus, Thermo Scientific, Waltham, Mass.) and mounted using Vectashield (Vector Laboratories, Burlingame, Calif.).

### Imaging and Analysis of Immunohistology

A Zeiss 710 confocal microscope with a 63×1.4 NA oil-immersion lens was used to analyze PSD-95 and SV2A staining. Three 40 μm hippocampus-containing sections were chosen, each 160 μm apart. Three images of the mossy fibers in the dentate gyrus were obtained for each slice and ImageJ used to calculate the area occupied by immunoreactive puncta and averaged per mouse. The same microscope was used to image GFAP and Iba1 staining. For GFAP and Iba1 staining, 3×2 20× tiled images were taken to capture the entirety of the hippocampus. This was done for each mouse for three sections from each mouse (160 μm apart). ImageJ was used to calculate the glial marker positive area and averaged per mouse. A Zeiss (Oberkochen, Germany) Axiolmager ZI fluorescent microscope (5× air-objective lens) was used to image and analyze β-amyloid (Aβ) staining, and the same imaging and analysis procedures were followed. In all cases, n=number of mice where the averaged % positive area for each derived from three 40 μm sections. All imaging and analyses were completed by an observer unaware of genotype.

### Preparation of Acute Mouse Brain Slices

Mouse brains from 2-6-month-old wild type (WT) or Pyk2−/− mice were dissected after rapid decapitation. Acute 400-μm coronal slices were cut in ice-cold artificial cerebral spinal fluid (aCSF: 119 mM NaCl; 2.5 mM KCl; 1.3 mM MgSO₄; 26.2 mM NaHCO₃; 11 mM D-glucose; 1.25 mM NaH₂PO₄) using a 1000 Plus VIBRATOME with steel razor blades. Brain slices were then transferred to brain slice submersion chambers filled with aCSF with 2.4 mM CaCl₂ at room temperature and incubated under constant oxygenation with 95% O₂ and 5% CO₂. Slices were allowed to recover for 45 min prior to any recording.

### Electrophysiology

400-μm coronal slices were prepared as described elsewhere herein and submerged in a recording chamber (BSC-PC, Warner Instruments) containing aCSF with 2.4 mM CaCl₂ that was continuously oxygenated (95% O₂, 5% CO₂). After the 45-min recovery period, slices were treated with Aβo (1 μM monomer, 10 nM oligomer estimate) or vehicle for 30 min prior to induction of LTP, with the identity of treatment and genotype blind to the electrophysiologist. Extracellular field recordings were performed in the Stratum radiatum of the hippocampus by stimulating Schaffer collateral fibers using a bipolar tungsten electrode (TM33CCNON, World Precision Instruments). Extracellular field excitatory post-synaptic potentials (fEPSPs) were recorded using a glass microelectrode (2-6 MS2) (4878, World Precision Instruments) filled with aCSF, and placed in the Stratum radiatum of CA1 neurons. Test stimuli were induced at 0.033 Hz, and the stimulus intensity was set at 50% of maximal fEPSP slope. A stable baseline was recorded for at least twenty minutes prior to induction of LTP, and slices were excluded if baseline could not be stabilized after 1 hour. LTP was induced by theta burst stimulation (10 bursts of 4 shocks at 100 Hz, with an interburst interval of 200 ms) given at baseline intensity. Long term depression (LTD) experiments were performed by delivering a low frequency stimulus (LFS) at 300 pulses (5 min) or 900 pulses (15 min) at 1 Hz. Slices were excluded from analysis with pre-established cutoff for poor preparations in which 10 or more data points below 75% of baseline level during the post-theta burst stimulation recording. Fewer than 13% of slices where excluded from any group. For LTD experiments, slices were excluded if potentials drifted by more than 20% in the period 30-60 min post LFS. Fewer than 17% of slices where excluded from any group. fEPSPs were recorded using an Axon Instruments 700B amplifier and a Digidata 1440A digitizer, with data analysis performed by pClamp 10 software (Molecular Devices) and Prism 7 software. All recording and analyses were completed by an observer unaware of genotype or treatment group.

### Behavioral Testing

The order of behavioral testing was Novel Object Recognition, Morris Water Maze, and Passive Avoidance Test last. Exclusion criteria were used for each test as described elsewhere herein. Exclusion criteria for one test was independent of any other test. All mouse handling and analyses were completed by an observer unaware of genotype.

Novel Object Recognition (NOR)

Mice were handled for five minutes a day for five days prior to experiment to reduce anxiety. Mice habituated to clean, rectangular, and empty rat cages in a dimly lit behavioral testing room for one hour. The cage was centered in front of the experimenter and oriented with the short axis perpendicular to the test administrator. During the acquisition trial, mice were removed from the behavioral cage, and two identical objects, either a single fifteen mL conical tube with orange cap or wrapped five mL plastic syringe (label side down), were placed one inch from the edge of either end of the long axis of the cage, perpendicular to the long axis. Object choice was pseudorandom; the object was recorded as the familiar object for each animal. The mouse was then replaced in cage's center, not facing either object, and a total timer counted up to ten minutes. The mice were allowed to explore the object and the time it took to accumulate thirty seconds of total orofacial object exploration was recorded. Orofacial was defined as whisking or sniffing. Finally, mice were left for ten minutes with the objects in the behavior cage. Then, the objects were removed and discarded and the mice were left in their cages for one hour. During the test trial, one of both the novel and familiar objects were placed on pseudorandom sides of the cage. Orofacial exploration of each object was timed until a combined total of thirty seconds was reached. After the trial and acquisition trial of each mouse, cages were cleaned to eliminate scent cues. The experimenter was blind to object novelty and genotype. Mice that did not explore both objects, that had barbered whiskers, or that failed to reach thirty seconds of exploration during either trial under six minutes were eliminated.

Morris Water Maze

Animals were assigned a random code and the experimenter was blinded to genotype. Each mouse was handled for 5 minutes for the 5 days leading up to any behavioral testing to reduce anxiety. Morris water maze testing was completed in three day blocks with probe trial performed on the fourth day. Mice were repeatedly placed in an open water pool about 1 meter in diameter to find a submerged hidden platform. Clear and colorless water remained at room temperature throughout all aspects of the experiment. The location of the platform remained fixed in the center of one of the quadrants (target quadrant) of the pool throughout the entire testing period. Visual cues remained constant throughout forward and reverse swims. The mice had a total of eight attempts per day to locate the platform, and training was divided into two blocks of four. The first block of four attempts was performed in the morning while the second block of four was performed in the afternoon. The order that the mice were tested in remained constant. The mice were gently placed into the pool, facing the wall, at one of four locations located in the opposite hemisphere from where the platform was and the latency to finding the platform was timed. The order of the four locations used to start the mice varied for each block to ensure that the mice would have to rely on spatial cues to find the platform. Once a mouse spent one second on the platform, the attempt was considered complete and the mouse would be removed from the pool. If a mouse did not find the platform within 60 seconds, it was guided to the platform and allowed to spend 10 seconds on the platform after which it was removed from the pool. This guiding was only performed for Trials 1 and 2. Twenty-four hours after the completion of the last block, the mice were tested in a probe trial. The probe trial consisted of returning the mice to the pool to explore for a single trial of 60 seconds with the hidden platform removed. The start location was the point in the pool furthest from where the platform originally was placed. The latency to platform testing and the probe trials were recorded on a JVC Everio G-series camcorder and tracked by Panlab's Smart software.

Passive Avoidance Test (PAT)

Following the administration of NOR and MWM, mice were subject to PAT using a box with equally sized, non-electrified light and electrified dark compartments. The guillotine door between the two collapsed with the mouse's complete movement from the light to the dark side. On Day One of testing, mice habituated to the light side for ninety seconds before the door opened. After this, the latency in seconds to enter the dark side was measured for up to three hundred seconds. Mice received a shock lasting two seconds with an intensity of 0.5 mA on the dark side and were left in the dark for ten seconds before returning to the home cage (Filali, et al., 2009, Brain Res. 1292:93-99). This was repeated after five minutes for each mouse. On Day Two, the shock was lowered to 0 mA, and the latency to enter the dark side after habituation was measured once more as a measure of retention of negative association. Perfect retention was considered the maximum latency of five minutes.

### Quantification and Statistical Analysis

One-way ANOVA with post-hoc Tukey pairwise comparisons, Repeated Measures ANOVA, and Student t test as specified in the Figure legends were performed using GraphPad Prism (version 5.0d) and SPSS Statistics (version 22). Means±sem and specific n values are reported in each Figure legend. Data are considered to be statistically significant if p<0.05. The assumption of Gaussian distribution was checked using D'Agostino-Pearson omnibus test.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing an Aβ-modulated disease or disorder in a mammal, the method comprising administering to the mammal a therapeutically effective amount of a Pyk2 inhibitor.

2. A method of preventing synaptic death, or improving synaptic survival, in a mammal afflicted with an Aβ-modulated disease or disorder, the method comprising administering to the mammal a therapeutically effective amount of a Pyk2 inhibitor.

3. The method of claim 1, wherein the Pyk2 inhibitor has a Ki≤100 nM against Pyk2.

4. The method of claim 1, wherein the Pyk2 inhibitor is selective for Pyk2 over at least one other kinase and/or enzyme.

5. The method of claim 1, wherein the Pyk2 inhibitor also inhibits Fyn.

6. The method of claim 1, wherein the Pyk2 inhibitor is selected from the group consisting of a nucleic acid, siRNA, antisense nucleic acid, ribozyme, peptide, antibody, small molecule, antagonist, aptamer, peptidomimetic, and any combinations thereof.

7. The method of claim 6, wherein the small molecule is selected from the group consisting of PF-719, tamatinib, foretinib, lestaurtinib, and Compounds 1-8, or a salt, solvate, tautomer, geometric isomer, enantiomer, or diastereoisomer thereof.

8. The method of claim 1, wherein the inhibitor is the only therapeutically effective agent administered to the mammal.

9. The method of claim 1, wherein the inhibitor is the only therapeutically effective agent administered to the mammal in an amount sufficient to treat or prevent an Aβ-modulated disease or disorder in the mammal, or prevent synaptic death, or improve synaptic survival, in a mammal afflicted with an Aβ-modulated disease or disorder.

10. The method of claim 1, wherein administration of the Pyk2 inhibitor provides a circulating Pyk2 inhibitor concentration of at least 100 nM in the mammal.

11. The method of claim 1, wherein the Aβ-modulated disease or disorder is selected from the group consisting of Alzheimer's Disease (AD), prodromal Alzheimer's Disease, amnestic mild cognitive impairment (MCI), Down syndrome dementia, traumatic brain injury, and frontotemporal dementia.

12. The method of claim 1, wherein the Aβ-modulated disease or disorder is AD.

13. The method of claim 1, wherein Aβ oligomer-induced signaling is inhibited in the mammal.

14. The method of claim 1, wherein the mammal is human.

15. The method of claim 1, wherein the Pyk2 inhibitor is administered to the mammal by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes.

16. The method of claim 1, further comprising administering to the mammal at least one additional agent that treats or prevents the Aβ-modulated disease or disorder in the mammal.

17. The method of claim 16, wherein the inhibitor and at least one additional agent are coformulated.

18. A kit for preventing or treating an Aβ-modulated disease, or preventing synaptic death or improving synaptic survival, in a mammal, the kit comprising a Pyk2 inhibitor, an applicator, and an instructional material for use thereof, wherein the instructional material recites the amount of, and frequency with which, the Pyk2 inhibitor is to be administered to the mammal.
